SEQUENCE LISTING (15 sequences)

SEQ ID NO: 1 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 3 - protein, 832 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 4 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 5 - protein, 701 aa, synthetic construct
MAIRRCWPRVVPGPALGWLLLLLNVLAPGRASPRLLDFPAPVCAQEGLSCRVKNSTCLDDSWIHPKNLTPSSPKNIYINLSVSSTQHGELVPVLHVEWTLQTDASILYLEGAELSVLQLNTNERLCVKFQFLSMLQHHRKRWRFSFSHFVVDPGQEYEVTVHHLPKPIPDGDPNHKSKIIFVPDCEDSKMKMTTSCVSSGSLWDPNITVETLDTQHLRVDFTLWNESTPYQVLLESFSDSENHSCFDVVKQIFAPRQEEFHQRANVTFTLSKFHWCCHHHVQVQPFFSSCLNDCLRHAVTVPCPVISNTTVPKPVADYIPLWVYGLITLIAILLVGSVIVLIICMTWRLSGADQEKHGLGSENLYFQYRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 6 - protein, 633 aa, synthetic construct
MGPQAAAGRMILLVVLMLSAKVGSGALTSTEDPEPPSVPVPTNVLIKSYNLNPVVCWEYQNMSQTPIFTVQVKVYSGSWTDSCTNISDHCCNIYEQIMYPDVSAWARVKAKVGQKESDYARSKEFLMCLKGKVGPPGLEIRRKKEEQLSVLVFHPEVVVNGESQGTMFGDGSTCYTFDYTVYVEHNRSGEILHTKHTVEKEECNETLCELNISVSTLDSRYCISVDGISSFWQVRTEKSKDVCIPPFHDDRKDSIWILVVAPLTVFTVVILVFAYWYTKKNSFKRKSIMLLGSENLYFQYRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 7 - protein, 620 aa, synthetic construct
MLSRLLPFLVTISSLSLEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQYFTVTNLSILVISMLLFCGILVCLVLQWYIRHPGKLPTVLVFKKPLGSENLYFQYRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 8 - protein, 592 aa, synthetic construct
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGLGSENLYFQLRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 9 - protein, 750 aa, synthetic construct
MPVSWFLLSLALGRNPVVVSLERLMEPQDTARCSLGLSCHLWDGDVLCLPGSLQSAPGPVLVPTRLQTELVLRCPQKTDCALCVRVVVHLAVHGHWAEPEEAGKSDSELQESRNASLQAQVVLSFQAYPIARCALLEVQVPADLVQPGQSVGSAVFDCFEASLGAEVQIWSYTKPRYQKELNLTQQLPDCRGLEVRDSIQSCWVLPWLNVSTDGDNVLLTLDVSEEQDFSFLLYLRPVPDALKSLWYKNLTGPQNITLNHTDLVPCLCIQVWSLEPDSERVEFCPFREDPGAHRNLWHIARLRVLSPGVWQLDAPCCLPGKVTLCWQAPDQSPCQPLVPPVPQKNATVNEPQDFQLVAGHPNLCVQVSTWEKVQLQACLWADSLGPFKDDMLLVEMKTGLNNTSVCALEPSGCTPLPSMASTRAARLGEELLQDFRSHQCMQLWNDDNMGSLWACPMDKYIHRRWVLVWLACLLLAAALFFFLLLKKDRRKAARGSRTALGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 10 - protein, 572 aa, synthetic construct
MLLVLLILAASCRSALPREPTIQCGSETGPSPEWMVQHTLTPGDLRDLQVELVKTSVAAEEFSILMNISWILRADASIRLLKATKICVSGKNNMNSYSCVRCNYTEAFQSQTRPSGGKWTFSYVGFPVELSTLYLISAHNIPNANMNEDSPSLSVNFTSPGCLNHVMKYKKQCTEAGSLWDPDITACKKNEKMVEVNFTTNPLGNRYTILIQRDTTLGFSRVLENKLMRTSVAIPVTEESEGAVVQLTPYLHTCGNDCIRREGTVVLCSETSAPIPPDDNRRMLGGWLPLFLVLLVAVWVLAAGIYLTWRQGRSTKTSFPISGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 11 - protein, 532 aa, synthetic construct
MRPLPLWLPSLLLCGLGAAASSPDSFSQLAAPLNPRLHLYNDEQILTWEPSPSSNDPRPVVYQVEYSFIDGSWHRLLEPNCTDITETKCDLTGGGRLKLFPHPFTVFLRVRAKRGNLTSKWVGLEPFQHYENVTVGPPKNISVTPGKGSLVIHFSPPFDVFHGATFQYLVHYWEKSETQQEQVEGPFKSNSIVLGNLKPYRVYCLQTEAQLILKNKKIRPHGLLSNVSCHETTANASARLQQVILIPLGIFALLLGLTGACFTLFLKYQSRVKYWFQAPPNIGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 12 - protein, 508 aa, synthetic construct
MSWAPSVAGWLGGFLLVPALGMIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPSWIVAIILIVSVLVVFLFLLGCFVVLWLIYKKTKHTFGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 13 - protein, 499 aa, synthetic construct
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 14 - protein, 371 aa, synthetic construct
MPEPAKSAPAPKKGSKKAVTKAQKKGGKKRKRSRKESYSIYVYKVLKQVHPDTGISSKAMGIMNSFVNDIFERIAGEASRLAHYNKRSTITSREIQTAVRLLLPGELAKHAVSEGTKAITKYTSAKDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2025/0248980 A1
(43) Pub. Date: Aug. 7, 2025

(54) IMMUNE MODULATORY AGENTS

(71) Applicant: PRESIDENT AND FELLOWS OF HARVARD COLLEGE, Cambridge, MA (US)

(72) Inventors: Jun R. HUH, Cambridge, MA (US); GUANGQING LU, Cambridge, MA (US)

(73) Assignee: PRESIDENT AND FELLOWS OF HARVARD COLLEGE, Cambridge, MA (US)

(21) Appl. No.: 18/853,224

(22) PCT Filed: Mar. 31, 2023
(86) PCT No.: PCT/US2023/065213

§ 371 Date: Oct. 1, 2024

Related U.S. Application Data
(60) Provisional application No. 63/326,410, filed on Apr. 1, 2022.

(51) Int. Cl. A61K 31/438; A61K 31/416; A61K 31/437; A61K 31/473; A61K 31/50; A61K 31/5386; A61K 31/55; A61K 45/06; A61P 37/02

(52) U.S. Cl. CPC A61K 31/438; A61K 31/416; A61K 31/437; A61K 31/473; A61K 31/50; A61K 31/5386; A61K 31/55; A61K 45/06; A61P 37/02

(57) ABSTRACT

The technology described herein is directed to immune modulatory agents and methods of use. In some aspects, described herein are agents that modulate IL-17a/IL-17 receptor-induced signalling and/or IL-17f/IL-17 receptor-induced signalling and associated methods of use thereof. In some aspects, described herein are agents that modulate TNF-α/TNF receptor signalling and associated methods of use thereof.

### CROSS-REFERENCE TO RELATED APPLICATIONS

This application claims benefit under 35 U.S.C. § 119 (e) of U.S. Provisional Application No. 63/326,410 filed Apr. 1, 2022, the contents of which are incorporated herein by reference in their entirety.

### SEQUENCE LISTING

The instant application contains a Sequence Listing which has been submitted in XML format via Patent Center and is hereby incorporated by reference in its entirety. Said XML copy, created on Mar. 30, 2023, is named 002806-191250WOPT_SL.xml and is 21,752 bytes in size.

### TECHNICAL FIELD

The technology described herein relates to immune modulatory agents.

### BACKGROUND

Immune cells often exert their effects by secreting cytokines. Cytokines then bind to their cognate receptors expressed on the membranes of target cells, binding of which initiates a downstream signaling cascade leading to various biological effects. Under pathological conditions, Th17 cells and IL-17a, and other cytokines can promote inflammatory and autoimmune diseases. Modulating cytokine activity with small molecules has therapeutic value not only in controlling autoimmunity but also in preventing and/or therapeutically treating certain types of neurodevelopmental disorders. There is currently an unmet need for immunomodulatory therapeutics for autoimmune and neurodevelopmental disorders.

### SUMMARY

The technology described herein is directed to immune modulatory agents and methods of use. In some aspects, described herein are agents that modulate IL-17a/IL-17a receptor-induced signalling and associated methods of use thereof. In some aspects, described herein are agents that modulate TNF-α/TNF-α receptor signalling and associated methods of use thereof.

In one aspect described herein is a method of modulating IL-17a/IL-17a receptor-induced signaling in a cell, the method comprising contacting the cell with an effective amount of at least one compound selected from the group consisting of compounds (1)-(11) and Formulas (I)-(III).

In some embodiments of any of the aspects, the at least one compound is selected from compounds (8), (9), and (10).

In some embodiments of any of the aspects, the at least one compound is selected from Formulas (I), (II), and (III).

In some embodiments of any of the aspects, the modulating comprises decreasing the IL-17a/IL-17a receptor signaling in the cell. In some embodiments of any of the aspects, the modulating comprises increasing the IL-17a/IL-17a receptor signaling in the cell.

In some embodiments of any of the aspects, the modulating is specific for IL-17a/IL-17a receptor signaling.

In some embodiments of any of the aspects, TNF-α/TNF-α receptor, IL-10/IL-10 receptor, IL-25/IL-25R receptor, and/or IFN-gamma/IFN-gamma receptor signaling in the cell is not modulated.

In one aspect described herein is a method of modulating TNF-α/TNF-α receptor-induced signaling in a cell, the method comprising contacting the cell with an effective amount of at least one compound selected from the group consisting of compounds (12)-(65) and Formulas (IV) and (V).

In some embodiments of any of the aspects, the at least one compound is selected from compounds (19) and (39).

In some embodiments of any of the aspects, the at least one compound is selected from Formulas (IV) and (V).

In some embodiments of any of the aspects, the modulating comprises decreasing the TNF-α/TNF-α receptor signaling in the cell. In some embodiments of any of the aspects, the modulating comprises increasing the TNF-α/TNF-α receptor signaling in the cell.

In some embodiments of any of the aspects, the modulating is specific for TNF-α/TNF-α receptor signaling.

In some embodiments of any of the aspects, IL-17a/IL-17a receptor, IL-10/IL-10 receptor, IL-25/IL-25R receptor, and/or IFN-gamma/IFN-gamma receptor signaling in the cell is not modulated.

In one aspect described herein is a method of modulating immune signaling in a cell, the method comprising contacting the cell with an effective amount of at least one compound selected from the group consisting of compounds (1)-(65) and Formulas (I)-(V).

In some embodiments of any of the aspects, the at least one compound is selected from compounds (8), (9), (10), (19), and (39).

In some embodiments of any of the aspects, the at least one compound is selected from Formulas (I), (II), (III), (IV), and (V).

In some embodiments of any of the aspects, the modulating comprises decreasing IL-17a/IL-17a receptor signaling in the cell.

In some embodiments of any of the aspects, the modulating comprises decreasing TNF-α/TNF-α receptor signaling in the cell.

In some embodiments of any of the aspects, the cell is an immune cell.

In some embodiments of any of the aspects, the cell expresses an IL-17 receptor or a TNF receptor.

In some embodiments of any of the aspects, the IL-17 receptor is IL-17RA and/or IL-17RC.

In some embodiments of any of the aspects, the TNF receptor is TNFR1 and/or TNFR2.

In some embodiments of any of the aspects, said modulating is in in vitro.

In some embodiments of any of the aspects, said modulating is in in vivo.

In some embodiments of any of the aspects, said modulating is in a subject in need of modulating an immune response.

In one aspect described herein is a method of modulating an immune response in a subject, the method comprising administering to a subject in need thereof an effective amount of at least one compound selected from the group consisting of compounds (1)-(65) and Formulas (I)-(V).

In some embodiments of any of the aspects, the at least one compound is selected from compounds (8), (9), (10), (19), and (39).

In some embodiments of any of the aspects, the at least one compound is selected from Formulas (I), (II), (III), (IV), and (V).

In some embodiments of any of the aspects, the subject has or is diagnosed with an IL-17a-associated disease or disorder or an IL-17f-associated disease or disorder.

In some embodiments of any of the aspects, the IL-17a-associated disease or disorder is selected from the group consisting of: rheumatoid arthritis, psoriasis, multiple sclerosis, systemic lupus erythematosus, Crohn's disease (CD), uveitis, ulcerative colitis, asthma, Behçet's disease, and hyper IgE syndrome.

In some embodiments of any of the aspects, the IL-17f-associated disease or disorder is recurrent urinary tract infections.

In some embodiments of any of the aspects, the subject has or is diagnosed with a TNF-α-associated disease or disorder.

In some embodiments of any of the aspects, the TNF-α-associated disease or disorder is selected from the group consisting of: rheumatoid arthritis, Crohn's disease, atherosclerosis, psoriasis, sepsis, diabetes, obesity, asthma, ulcerative colitis, ankylosing spondylitis, and moderate to severe plaque psoriasis.

In some embodiments of any of the aspects, the subject has or is diagnosed with an inflammatory disease or disorder or an autoimmune disease.

In one aspect described herein is a method of treating an inflammatory disease or disorder or an autoimmune disease, the method comprising administering to a subject in need thereof an effective amount of at least one compound selected from the group consisting of compounds (1)-(65) and Formulas (I)-(V).

In some embodiments of any of the aspects, the at least one compound is selected from compounds (8), (9), (10), (19), and (39).

In some embodiments of any of the aspects, the at least one compound is selected from Formulas (I), (II), (III), (IV), and (V).

In some embodiments of any of the aspects, the inflammatory disease or disorder is acute or chronic.

In some embodiments of any of the aspects, the inflammatory disease or disorder is selected from the group consisting of arthritis, asthma, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves' disease, gastrointestinal allergies, conjunctivitis, atherosclerosis, coronary artery disease, angina, and small artery disease.

In some embodiments of any of the aspects, the autoimmune disease is selected from the group consisting of glomerulonephritis, Goodpasture's syndrome, necrotizing vasculitis, lymphadenitis, peri-arteritis nodosa, systemic lupus erythematosus, rheumatoid, arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barre syndrome, Hashimoto's thyroiditis, and cardiomyopathy.

In one aspect described herein is a method of treating an IL-17a-associated disease or disorder, the method comprising administering to a subject in need thereof an effective amount of at least one compound selected from the group consisting of compounds (1)-(11) and Formulas (I)-(III).

In some embodiments of any of the aspects, the at least one compound is selected from compounds (8), (9), and (10).

In some embodiments of any of the aspects, the at least one compound is selected from Formulas (I), (II), and (III).

In some embodiments of any of the aspects, the IL-17a-associated disease or disorder is selected from the group consisting of: rheumatoid arthritis, psoriasis, multiple sclerosis, systemic lupus erythematosus, Crohn's disease (CD), uveitis, ulcerative colitis, asthma, Behçet's disease, and hyper IgE syndrome.

In one aspect described herein is a method of treating an IL-17f-associated disease or disorder, the method comprising administering to a subject in need thereof an effective amount of at least one compound selected from the group consisting of compounds (1)-(11) and Formulas (I)-(III).

In some embodiments of any of the aspects, the at least one compound is selected from compounds (8), (9), and (10).

In some embodiments of any of the aspects, the at least one compound is selected from Formulas (I), (II), and (III).

In some embodiments of any of the aspects, the IL-17f-associated disease or disorder is recurrent urinary tract infections.

In one aspect described herein is a method of treating a TNF-α-associated disease or disorder, the method comprising administering to a subject in need thereof an effective amount of at least one compound selected from the group consisting of compounds (12)-(65) and Formulas (IV) and (V).

In some embodiments of any of the aspects, the at least one compound is selected from compounds (19) and (39).

In some embodiments of any of the aspects, the at least one compound is selected from Formulas (IV) and (V).

In some embodiments of any of the aspects, the TNF-α-associated disease or disorder is selected from the group consisting of: rheumatoid arthritis, Crohn's disease, atherosclerosis, psoriasis, sepsis, diabetes, obesity, asthma, ulcerative colitis, ankylosing spondylitis, and moderate to severe plaque psoriasis.

In one aspect described herein is a method of treating a neurological disease or disorder, the method comprising administering to a subject in need thereof an effective amount of at least one compound selected from the group consisting of compounds (1)-(65) and Formulas (I)-(V).

In some embodiments of any of the aspects, the at least one compound is selected from compounds (8), (9), (10), (19), and (39).

In some embodiments of any of the aspects, the at least one compound is selected from Formulas (I), (II), (III), (IV), and (V).

In some embodiments of any of the aspects, the neurological disease or disorder is selected from autism spectrum disorder, schizophrenia, depression, and bipolar disorder.

In some embodiments of any of the aspects, the neurological disease or disorder is associated with abnormal behavioral phenotypes and/or abnormal cortical phenotypes in the central nervous system.

In some embodiments of any of the aspects, the neurological disease or disorder is associated with an autism-like phenotype.

In some embodiments of any of the aspects, the method further comprises administering an anti-inflammatory agent to the subject.

In some embodiments of any of the aspects, the method further comprises administering an immunomodulatory agent to the subject.

In some embodiments of any of the aspects, the compound is administered orally or parenterally to the subject.

In one aspect described herein is a pharmaceutical composition comprising at least one compound selected from the group consisting of compounds (1)-(65) and Formulas (I)-(V) and a pharmaceutically acceptable carrier.

In some embodiments of any of the aspects, the at least one compound is selected from compounds (8), (9), (10), (19), and (39).

In some embodiments of any of the aspects, the at least one compound is selected from Formulas (I), (II), (III), (IV), and (V).

In some embodiments of any of the aspects, the pharmaceutical composition is formulated in a form selected from the group consisting of tablets, gel capsules, sugar-coated tablets, syrups, suspensions, solutions, powders, lyophilized powders, granules, emulsions, lipid particles, polymeric particles, and controlled release compositions.

In some embodiments of any of the aspects, the pharmaceutical composition is formulated for parenteral or oral administration.

### BRIEF DESCRIPTION OF THE DRAWINGS

FIG. 1 is a dot plot showing the identification of small-molecule compounds that reduce IL-17A or TNF-a receptor activities. Small-molecule screens were performed with 134K compounds. Individual compounds were added to IL-17A reporters or TNF-a reporters after adding respective ligands such as IL-17A or TNF-a. The reporter cells were then incubated for 48 hours before subjecting them to microscopic analyses. Anti-IL17A and anti-TNFa antibodies were used as the positive controls.

FIG. 2 is a schematic showing the structures of compounds inhibiting IL-17 receptor reporter activities. Identified compounds exhibited selective effects on IL-17R but not on IFNg-R, IL-10R, and TNFa-R reporters.

FIG. 3 is a schematic showing the structures of compounds inhibiting TNFa receptor reporter activities. Identified compounds exhibited selective effects on TNFa-R but not on IFNg-R, IL-10R, IL-25R and IL-17R reporters.

FIG. 4 is a schematic showing the overall screen process. Initial screens with 134K compounds were performed simultaneously using IL-17R and TNF-R1 reporter cells. 139 and 352 cherry-pick of IL17R and TNFR1 reporter cells were selected and subjected to additional screens against other cytokine receptor reporters (e.g., IL25R/IL10R/IFNgR reporters). 11 and 54 hits were identified as IL17R and TNFR1 specific inhibitors.

FIG. 5 is a dot plot showing the identification of small-molecule compounds that reduce IL-17A or TNF-a receptor activities. Small-molecule screens were performed with 114,665 compounds. Individual compounds were added to IL-17A-reporters or TNF-a-reporters following the addition of respective ligands such as IL-17A or TNF-a (25 ng/ml). The reporter cells were then incubated for additional 48 hours before subjecting them for microscopic analyses (GFP percentage was measured by microscope). Anti-IL17A and anti-TNFa antibodies were used as the positive controls.

FIG. 6 is a schematic showing the structures of compounds which reduce IL-17A-receptor reporter activities, but not TNF-a-receptor reporter activities. Initial hits in FIG. 5 were subjected to additional screens against other cytokine receptor reporters (e.g., other than IL-17A or TNF-a). Shown here in FIG. 6 are those compounds that displayed IL-17A-reporter-specific activities.

FIG. 7 is a schematic showing the structures of compounds which reduce TNF-a-receptor reporter activities, but not IL-17A-receptor reporter activities. Initial hits in FIG. 5 were subjected to additional screens against other cytokine receptor reporters (e.g., other than IL-17A or TNF-a). Shown here in FIG. 7 are those that display TNF-a-reporter-specific activities.

FIG. 8 is a series of line graphs showing the blocking specificity of six hits, including 5 IL17A hits (compounds 1, 3, 5, 8, and 2) and 1 TNFa hit (compound 23). Different concentrations of compounds were used to block IL17A-, TNFa-, IL10-, or IFNg-receptor reporters, upon treatment of their respective ligands. The GFP percentage was measured by microscope.

FIG. 9 shows structures of compounds reducing IL-17A-, not TNF-a-, receptor reporter activities. Initial IL-17A-receptor reporter (IL-17RA/IL-17RC) hits were subjected to additional screens against other cytokine receptor reporters. Shown in FIG. 9 are those compounds that displayed IL-17A-receptor reporter-specific activities. Compounds highlighted with boxes in FIG. 9 (compounds 2, 3, 8, 9, and 10) are those that exhibited inhibitory activities in a secondary assay (see e.g., FIG. 11). IC₅₀ and Eₘₐₓ of the compounds reducing IL-17A-receptor reporter activities are shown in Table 1, Table 2, and Table 9.

FIG. 10 shows structures of compounds reducing TNF-a-, not IL-17A-, receptor reporter activities. Initial hits in were subjected to additional screens against other cytokine receptor reporters. Shown here in FIG. 10 are those compounds that displayed TNF-a-reporter-specific activities. Compounds highlighted in black boxes in FIG. 10 (compounds 15, 19, 21, 28, 32, 39, and 43) were confirmed to have inhibitory effects against some of TNF-a downstream genes at the secondary assays (see e.g., FIG. 12). Those compounds highlighted in grey boxes in FIG. 10 (compounds 19, 24, 34, 39, 51, and 60, particularly C18 and C19, corresponding to compounds 24 and 51, respectively) have amendable chemical structures for derivatization. IC₅₀ and Eₘₐₓ of the compounds reducing TNF-a-receptor reporter activities are shown in Table 3, Table 4, and Table 10.

FIG. 11 is a bar graph showing that small molecule compounds reduced IL17A-dependent gene expression in mouse keratinocytes. Mouse keratinocytes were cultured and stimulated with 5 ng/ml IL17A for 24 hours. During the treatment, 10 μM of each compound was added, and reverse transcription-quantitative polymerase chain reaction (RT-qPCR) was used for the gene expression level analyses of the following genes: cxcl1, cxcl2, cxcl5, 24p3, zc3h12a, S100a8, and S100a9. The top-down order of the treatment conditions indicated in the legend correspond to the left-right order of the bars for each gene. Compounds C999-860 (8), C881-0413 (9), LAS51593897 (11), and LAS51595730 (10) (highlighted by boxes) led to robust inhibitory effects, similar to IL-17RA blocking antibodies. Compounds C770-0711 (2), C770-0720 (3), 60546479 (6), C881-0413 (9), and LAS 51593897 (11) are also of interest (see grey boxes).

FIG. 12A-12C are dots plots showing that small molecule compounds reduced TNF-a-dependent gene expression in three different cell lines. THP-1 (FIG. 12A), 293T (FIG. 12B), and HepG2 (FIG. 12C) cells were cultured and stimulated with 5 ng/ml TNF-a for 24 hours. During the treatment, 10 μM of each compound was added. RT-qPCR was used to analyze the expression levels of selected TNF-a downstream genes (IL1b, CCL4 and CXCL1 in THP-1 cells in FIG. 12A; IL18, VCAM, and CXCL10 in 293T cells in FIG. 12B; and IL18, CXCL1, and CXCL5 in HepG2 cells in FIG. 12C). C5:C509-0242 (highlighted in grey; compound 39) led to robust inhibitory effects, similar to TNF-a blocking antibodies. C6:D262-1066, b:LAS51616998 and d:LAS52468797 (highlighted in black; compounds 19, 32, and 21, respectively) led to robust inhibitory effects in THP-1 cells, while they showed enhanced effects in 293T and HepG2 cells.

FIG. 13 shows selected compounds. Three compounds reduce IL-17A-, not TNF-a-, receptor reporter activities: C999-860 (8), C881-0413 (9), and LAS 51595730 (10). Two compounds reduce TNF-a-, not IL-17A-, receptor reporter activities: C5: C509-0242 (39) and C6: D262-1066 (19).

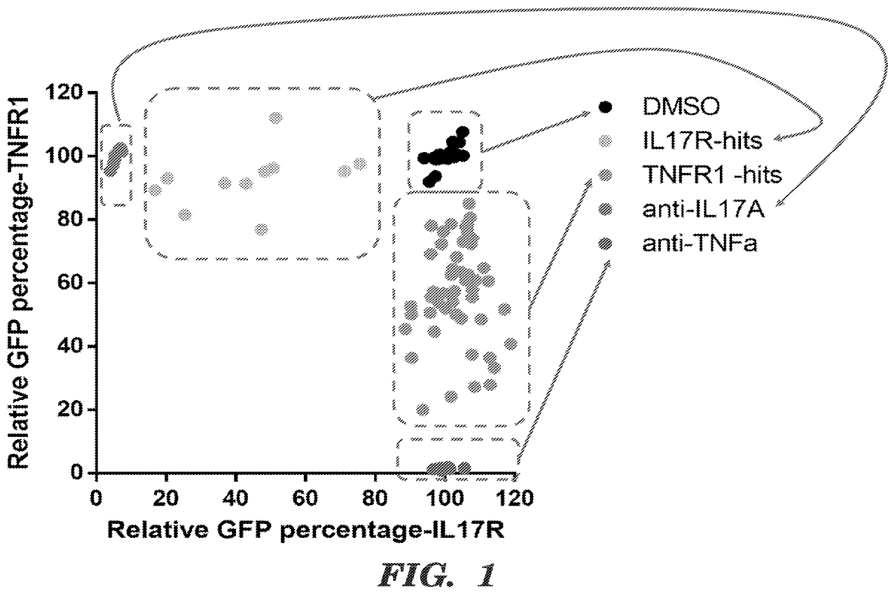

### DETAILED DESCRIPTION

The technology described herein is directed to immune modulatory agents and methods of use. In some aspects, described herein are agents that modulate IL-17a/IL-17 receptor-induced signalling and/or IL-17f/IL-17 receptor-induced signalling and associated methods of use thereof. In some aspects, described herein are agents that modulate TNF-α/TNF receptor signalling and associated methods of use thereof.

### Compounds

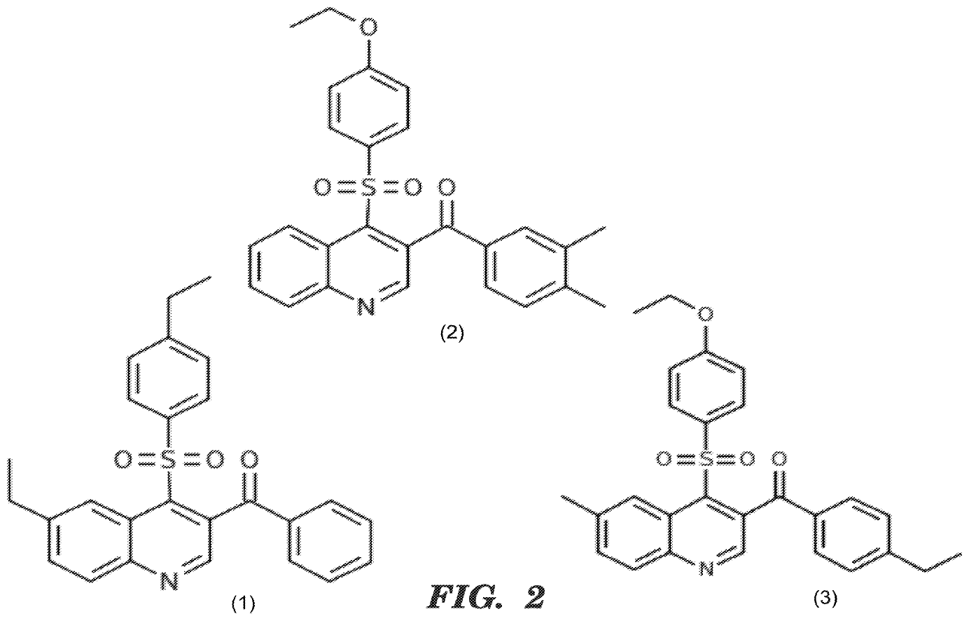

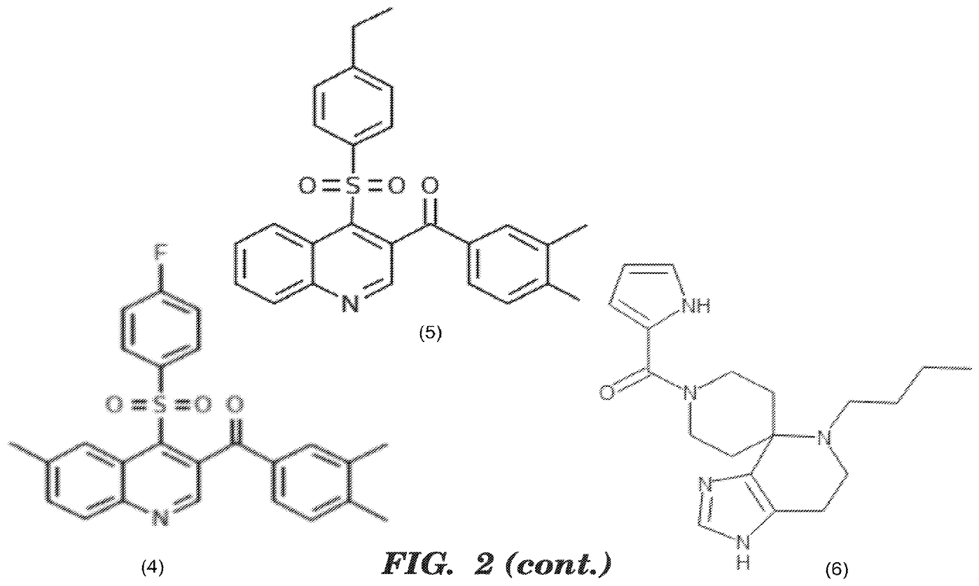

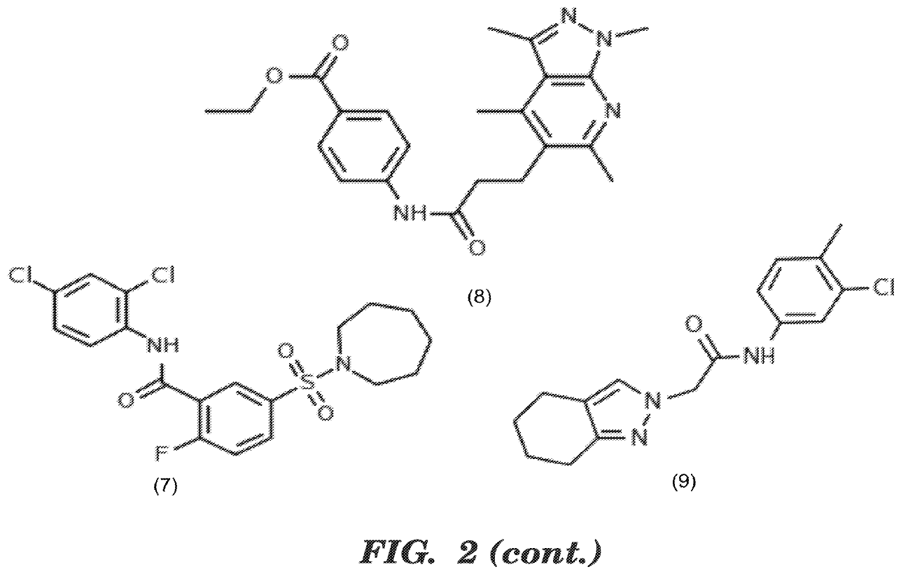

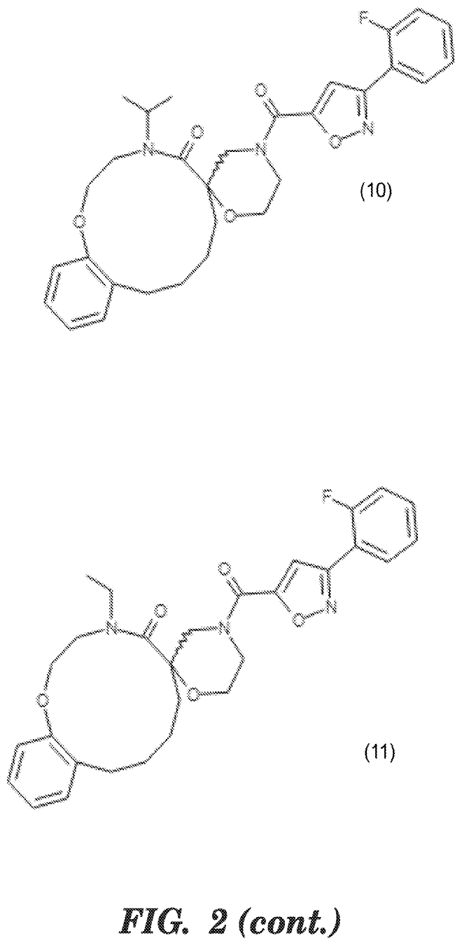

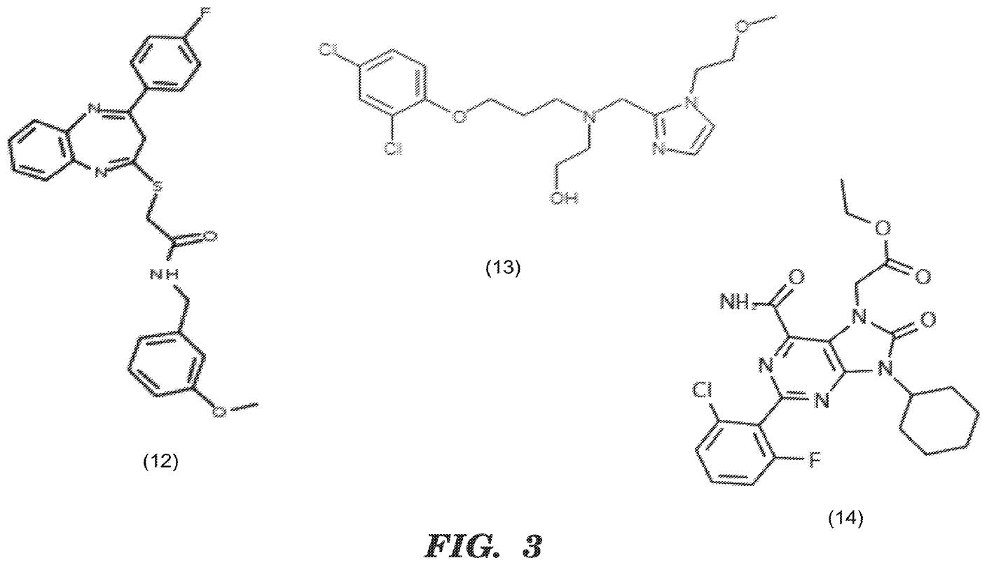

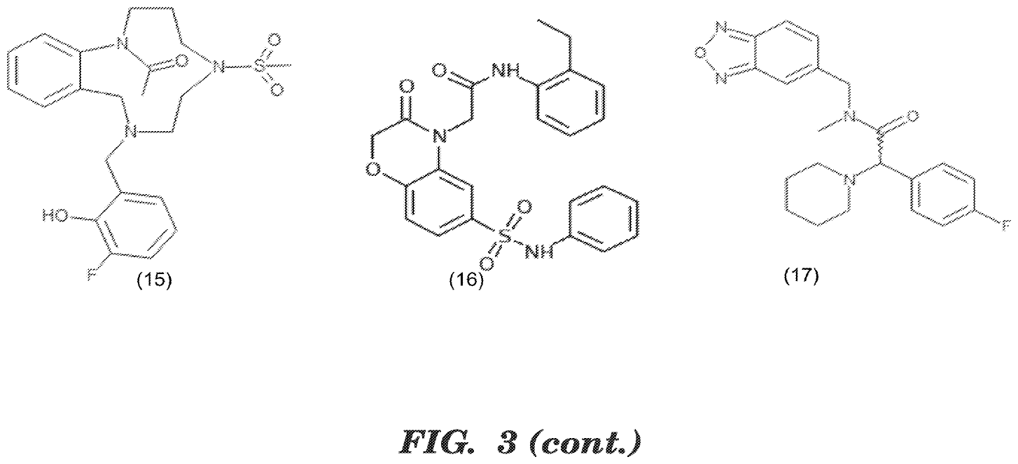

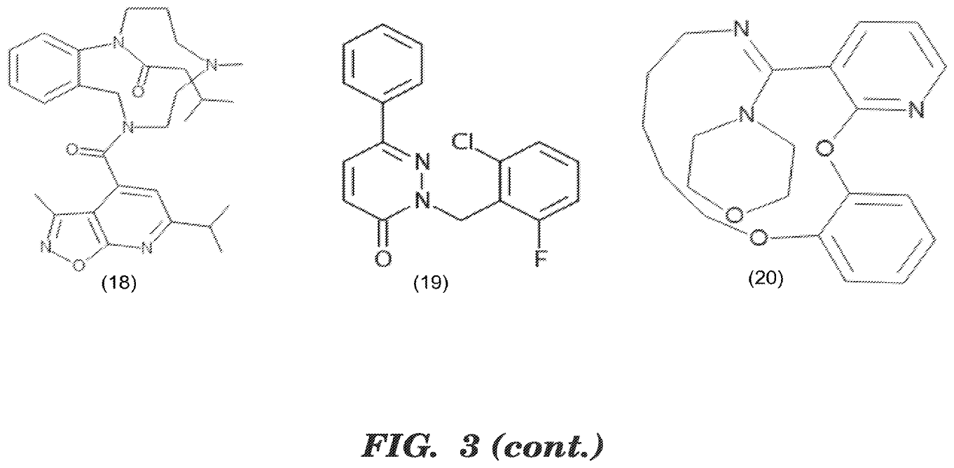

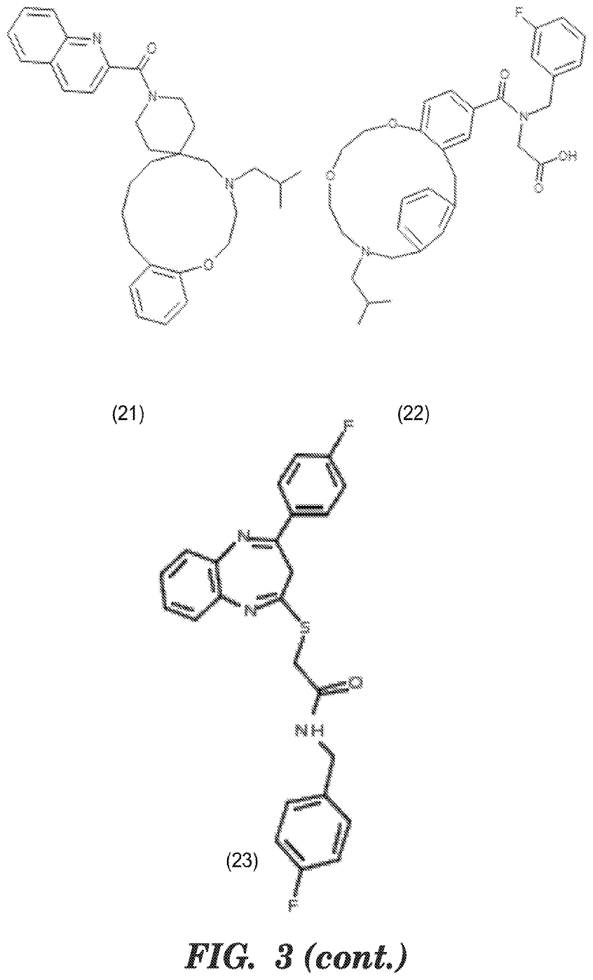

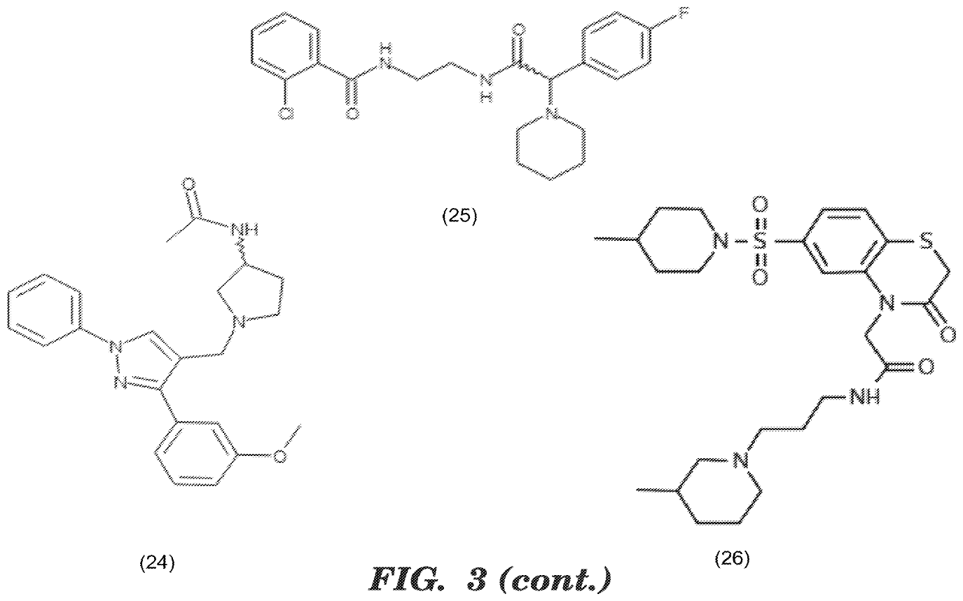

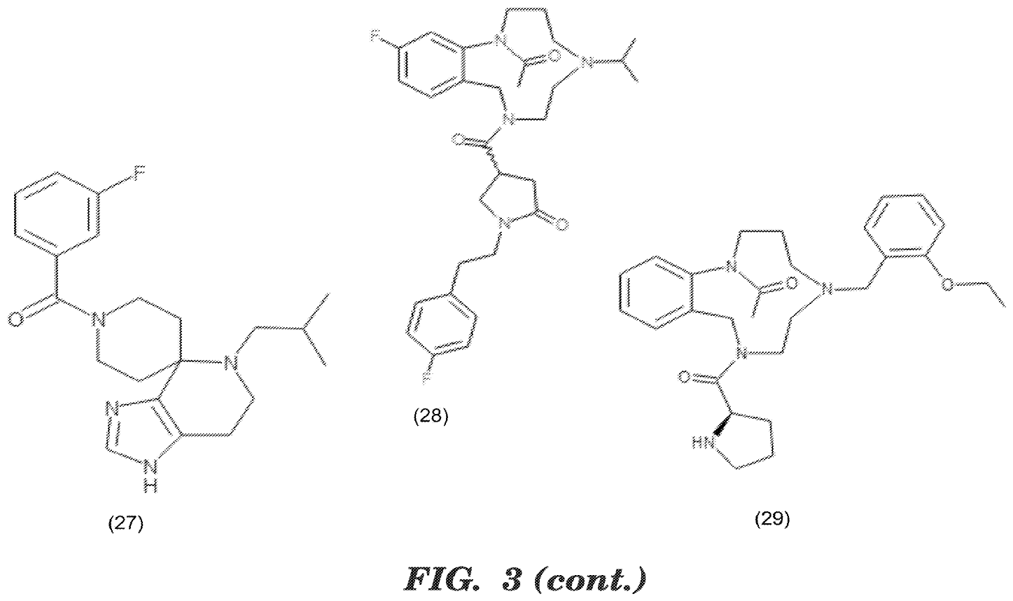

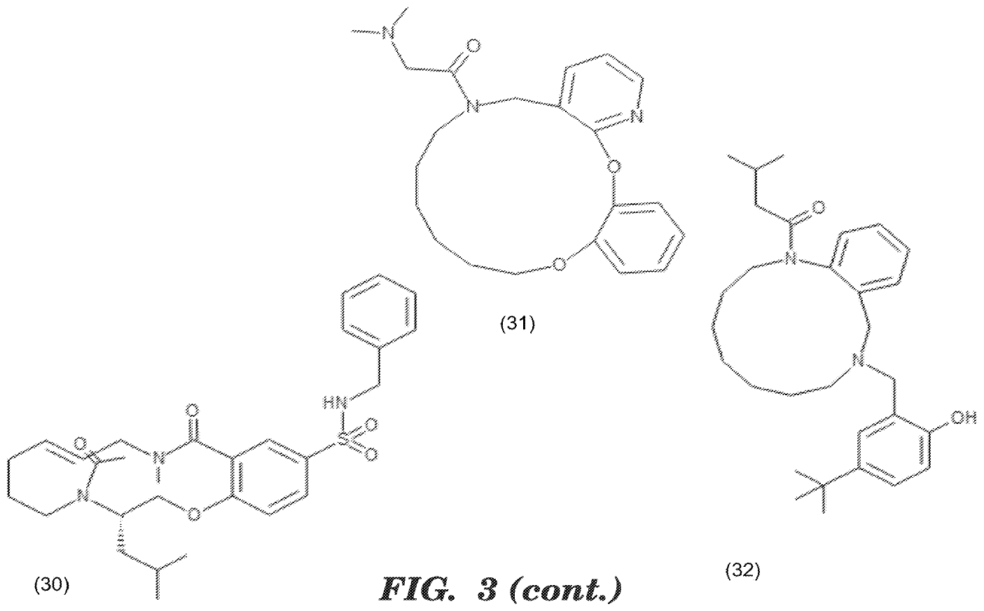

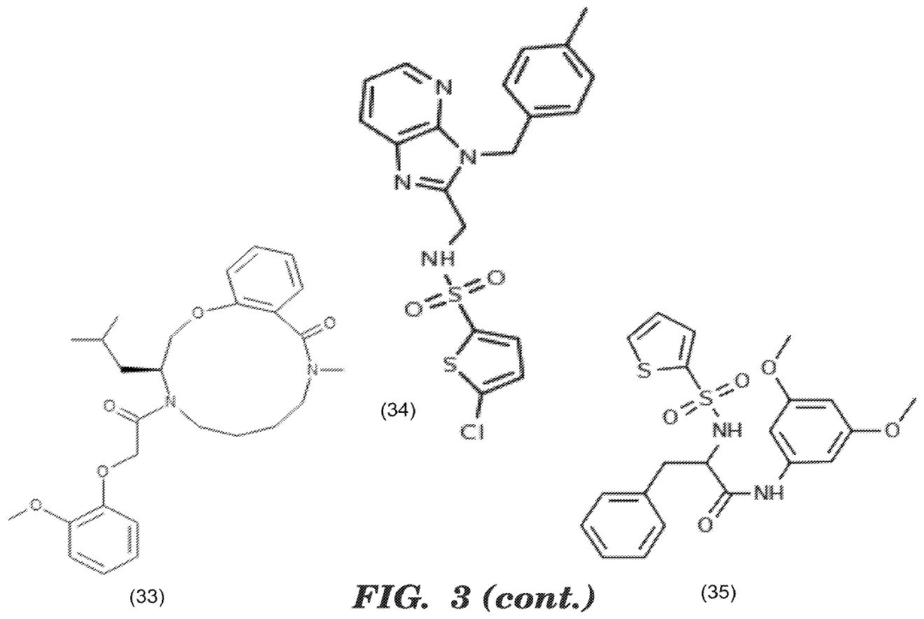

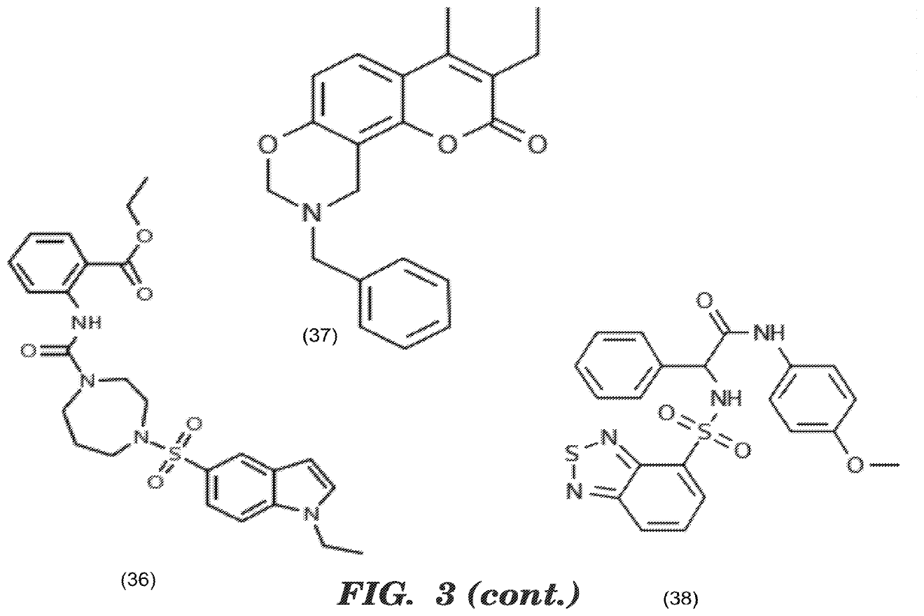

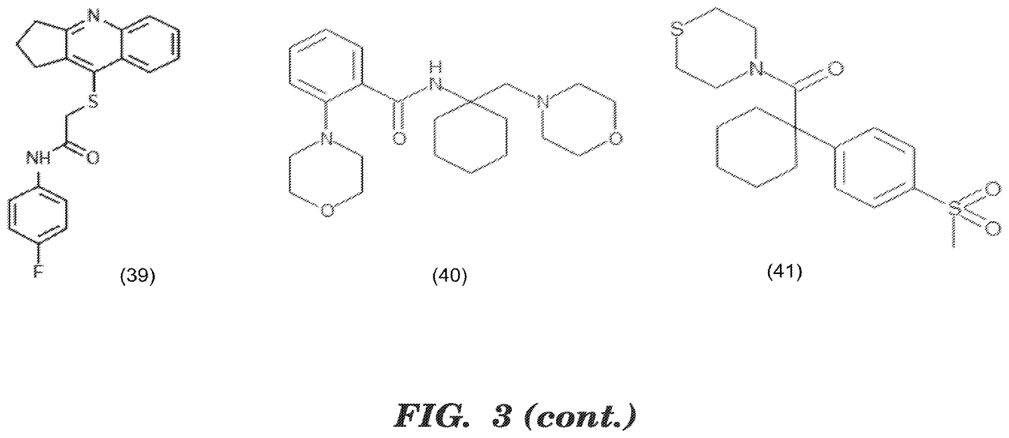

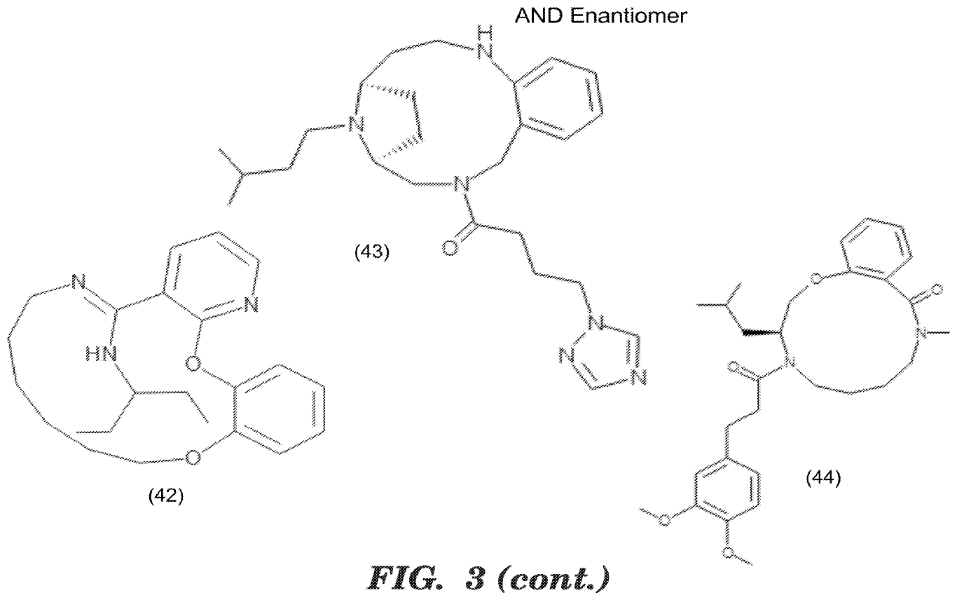

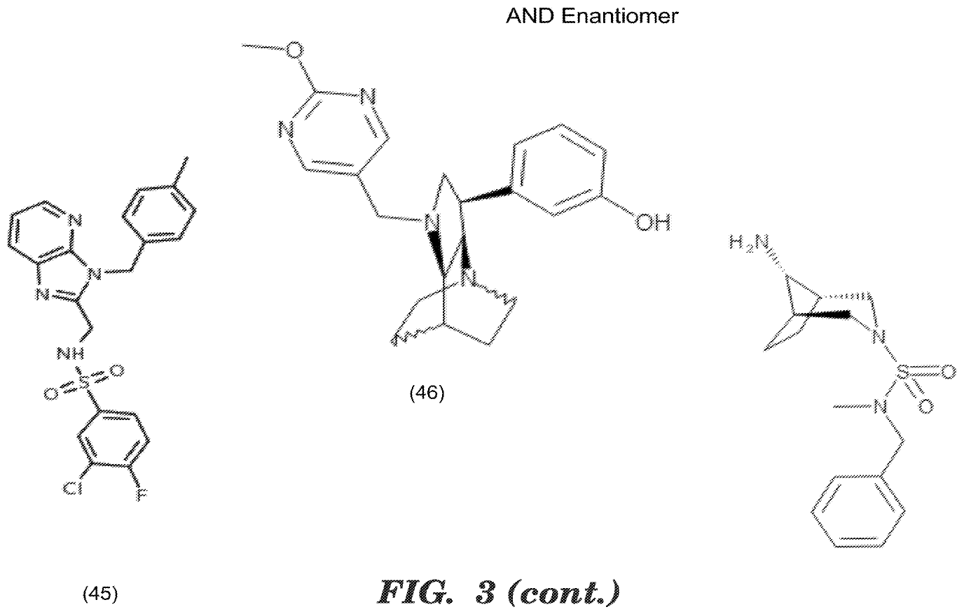

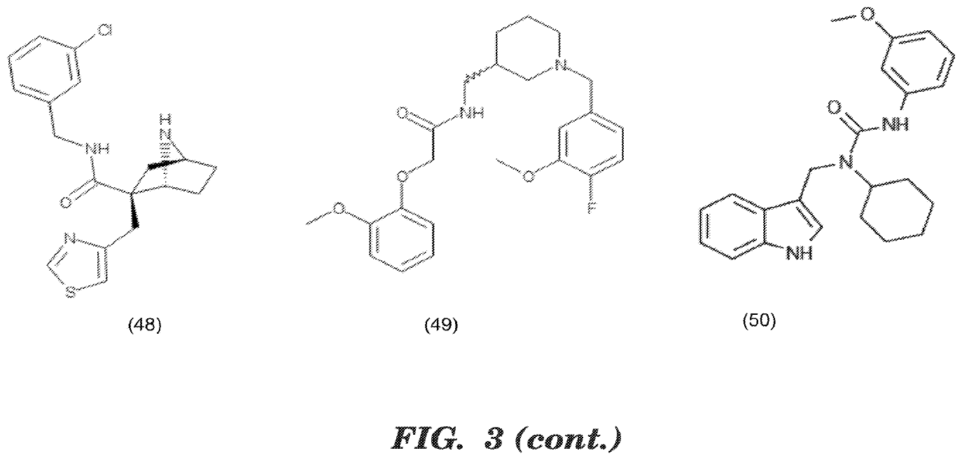

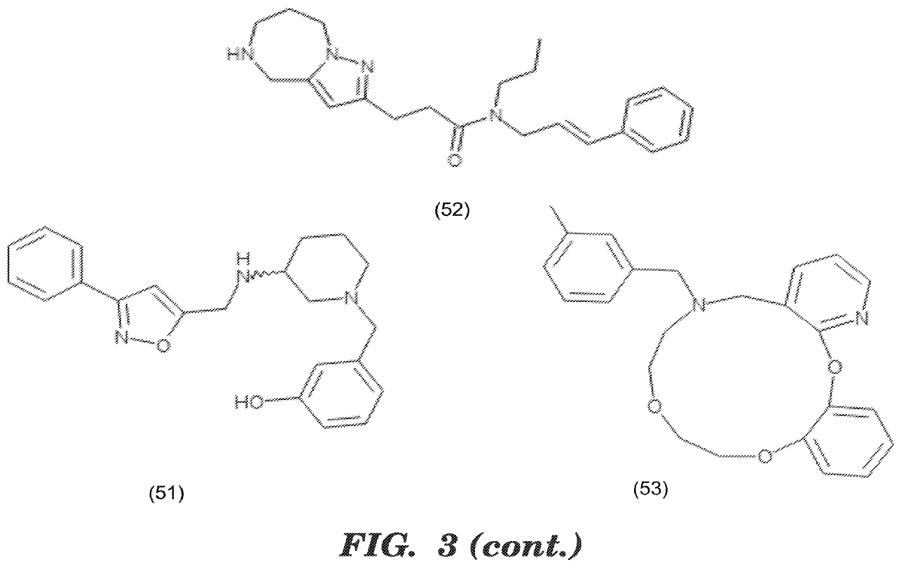

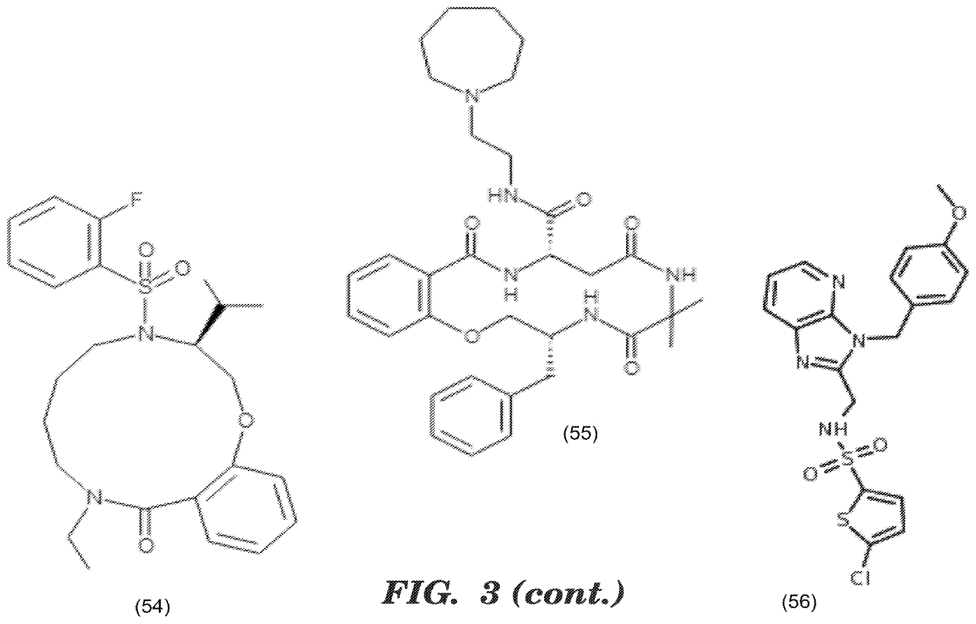

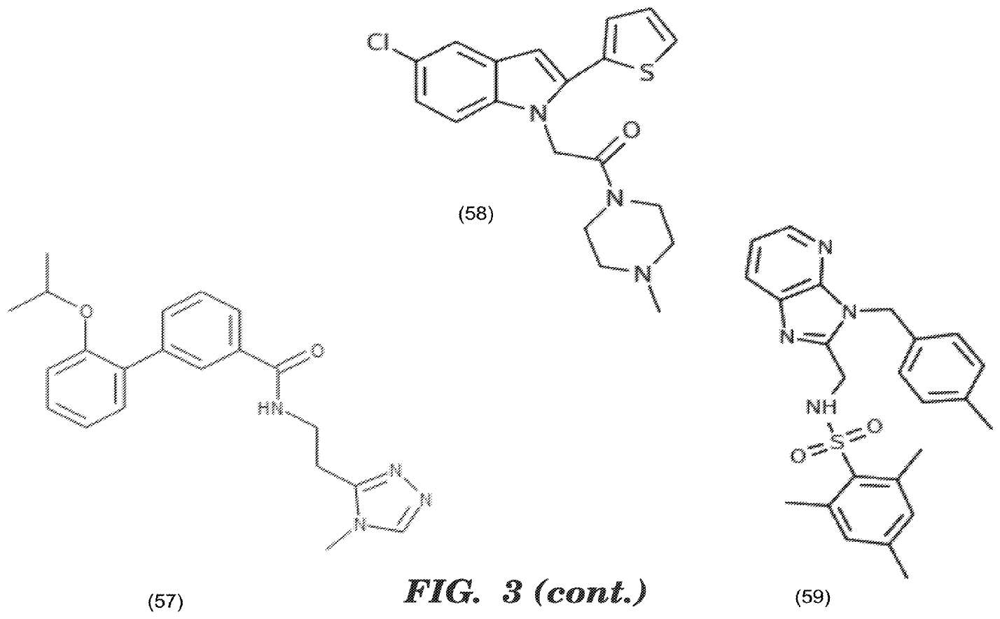

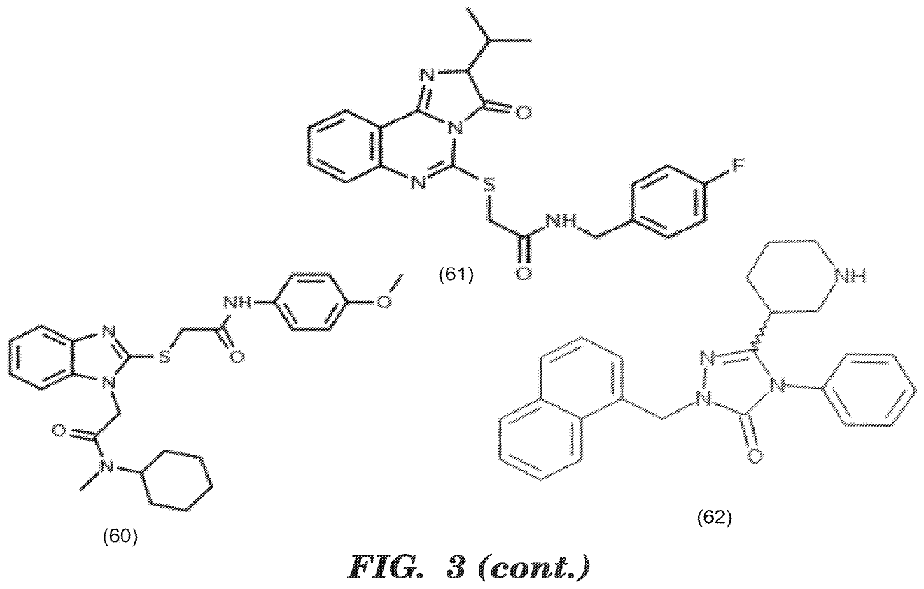

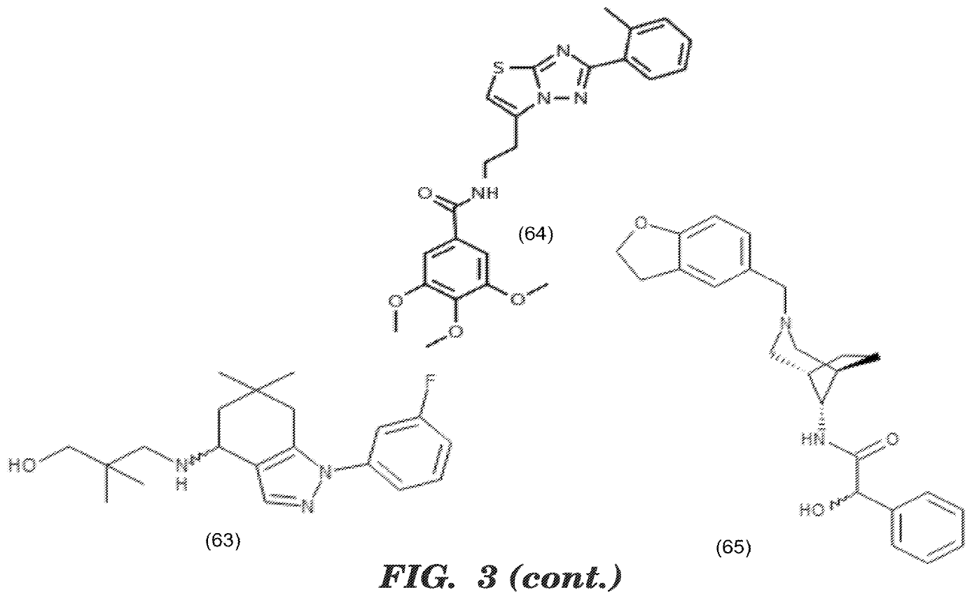

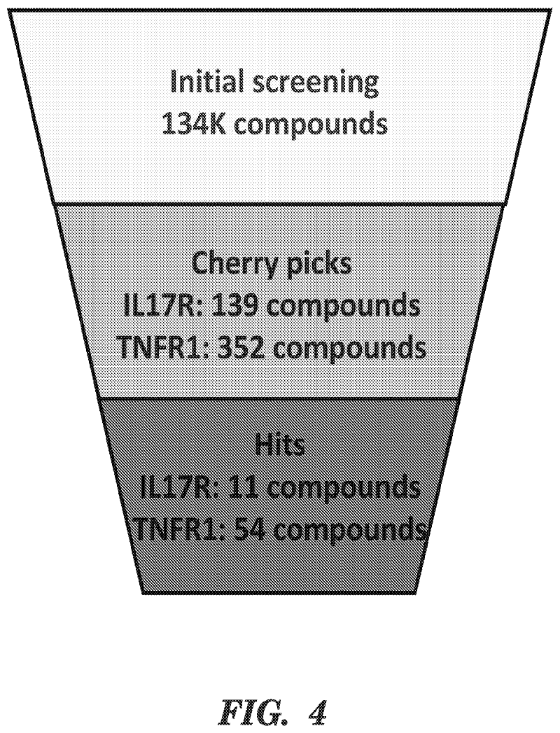

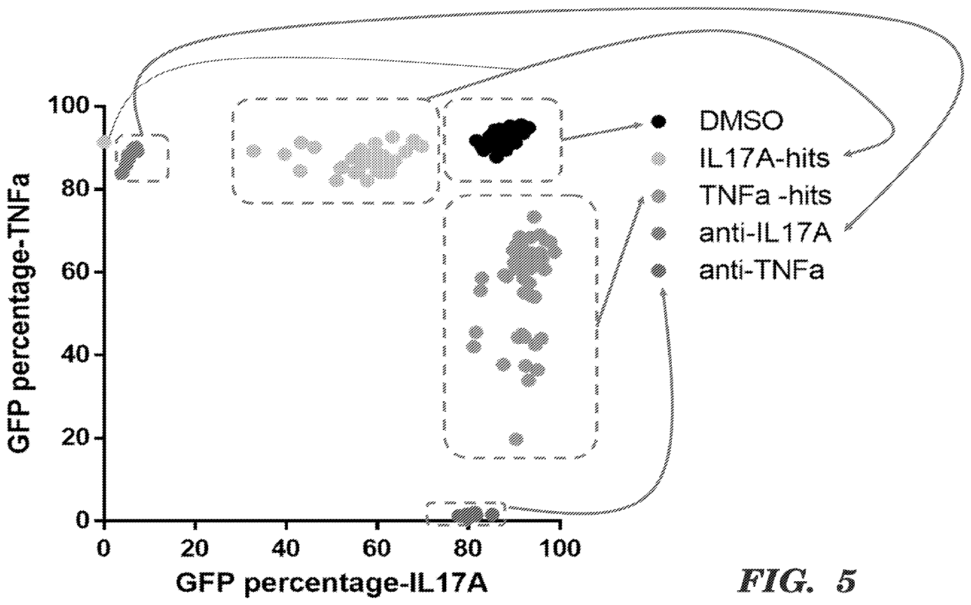

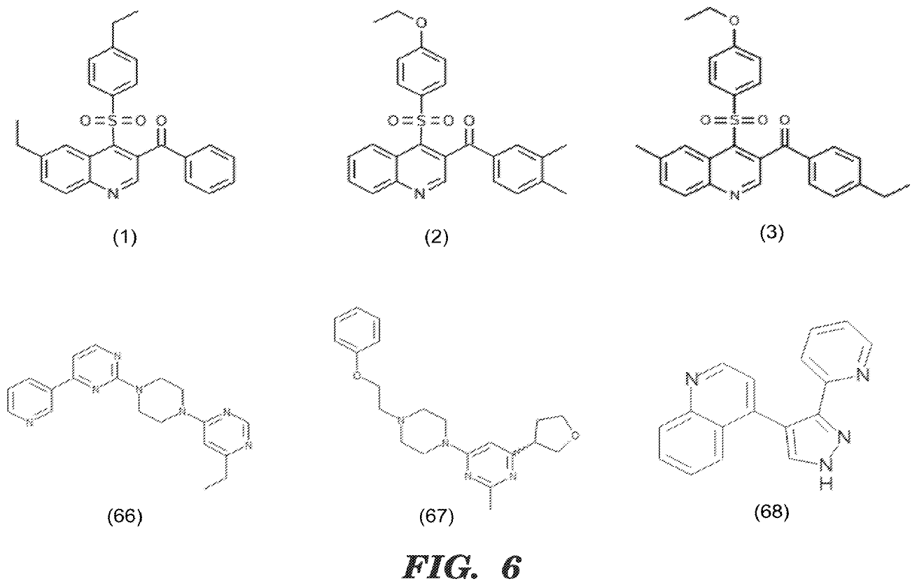

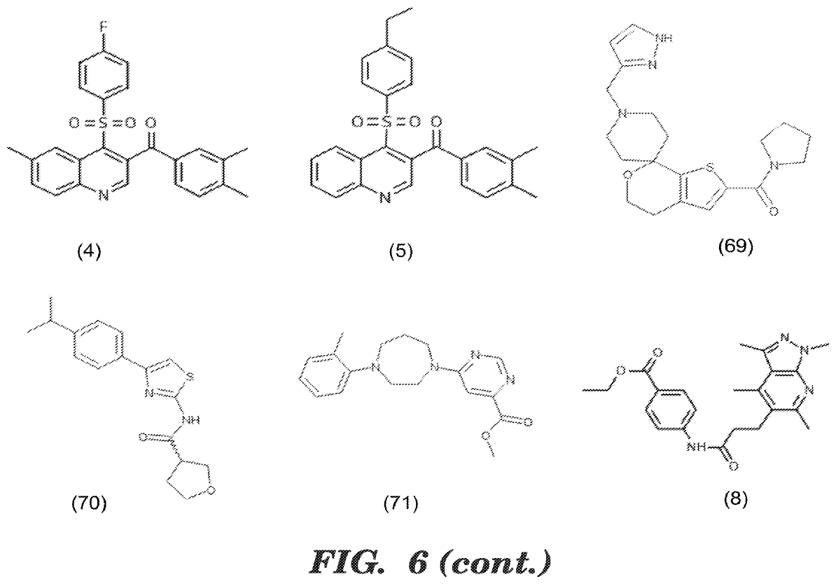

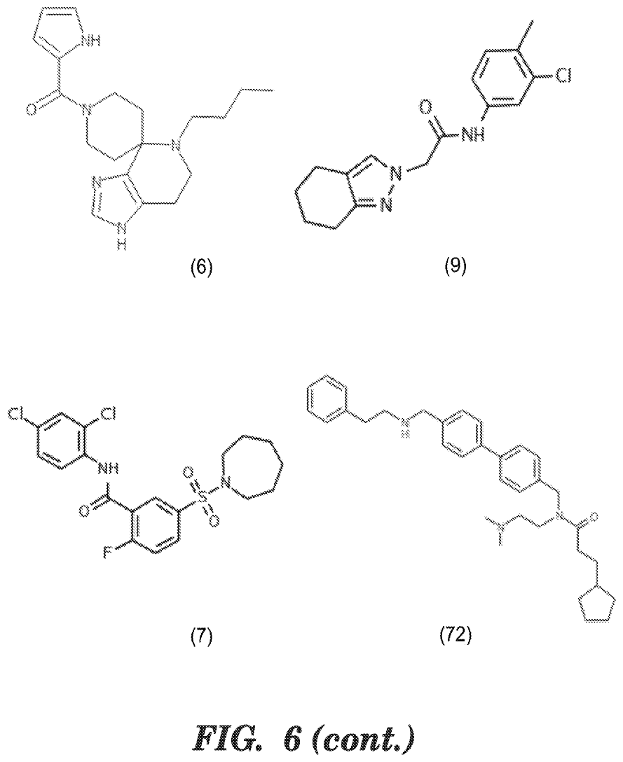

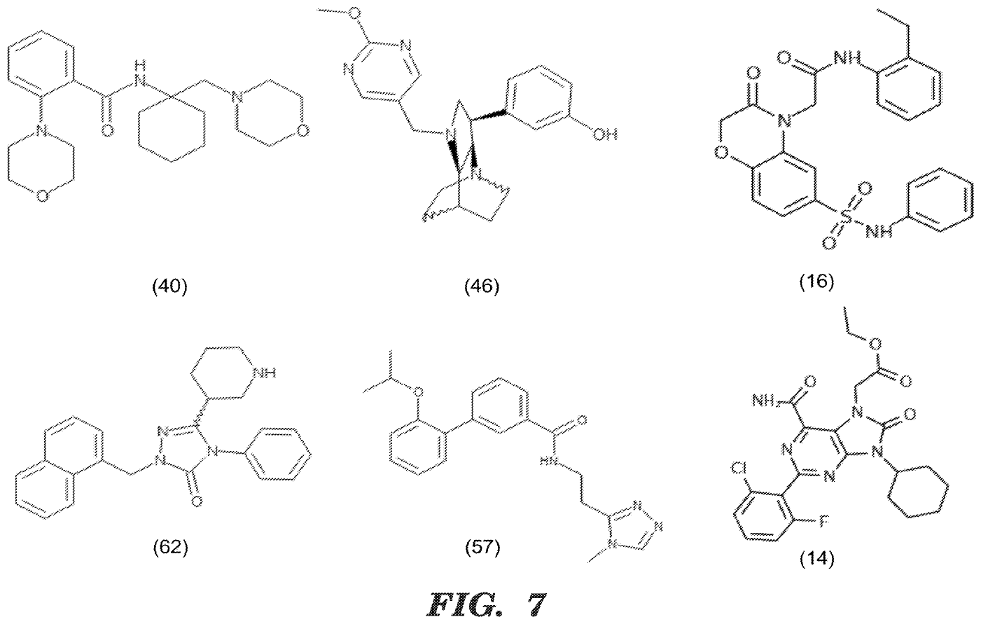

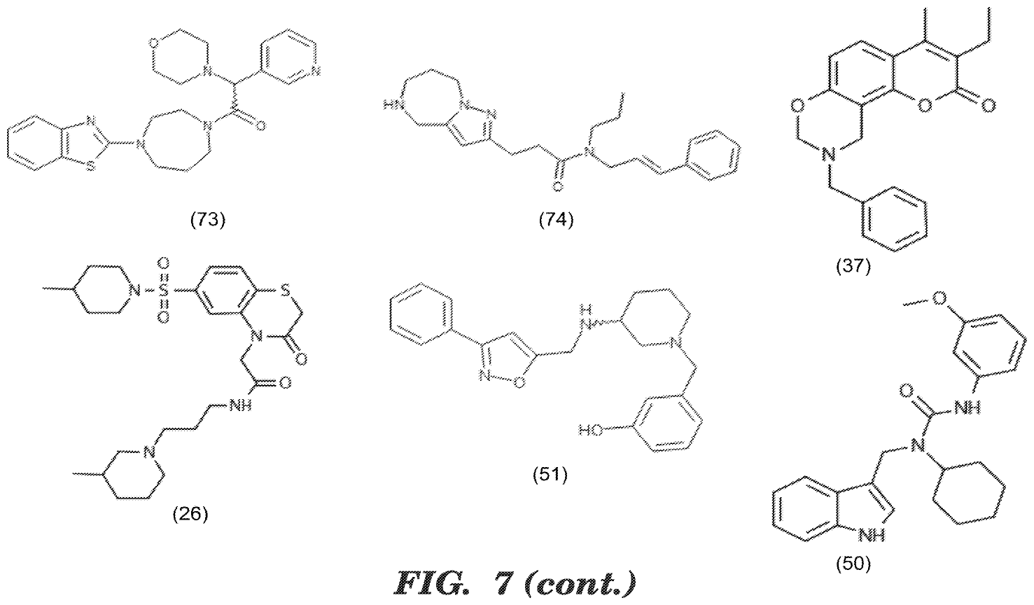

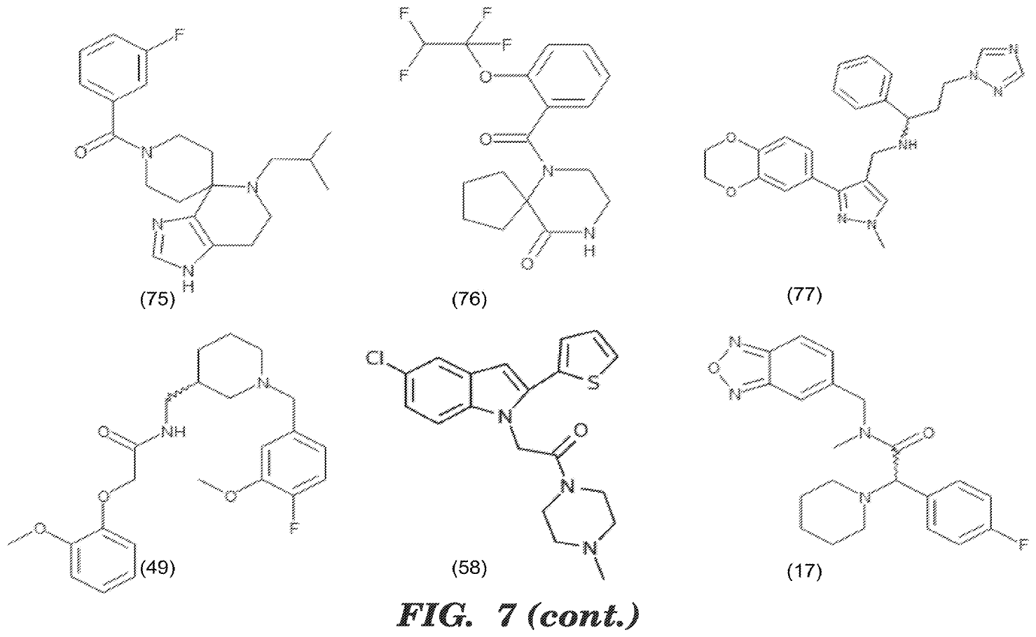

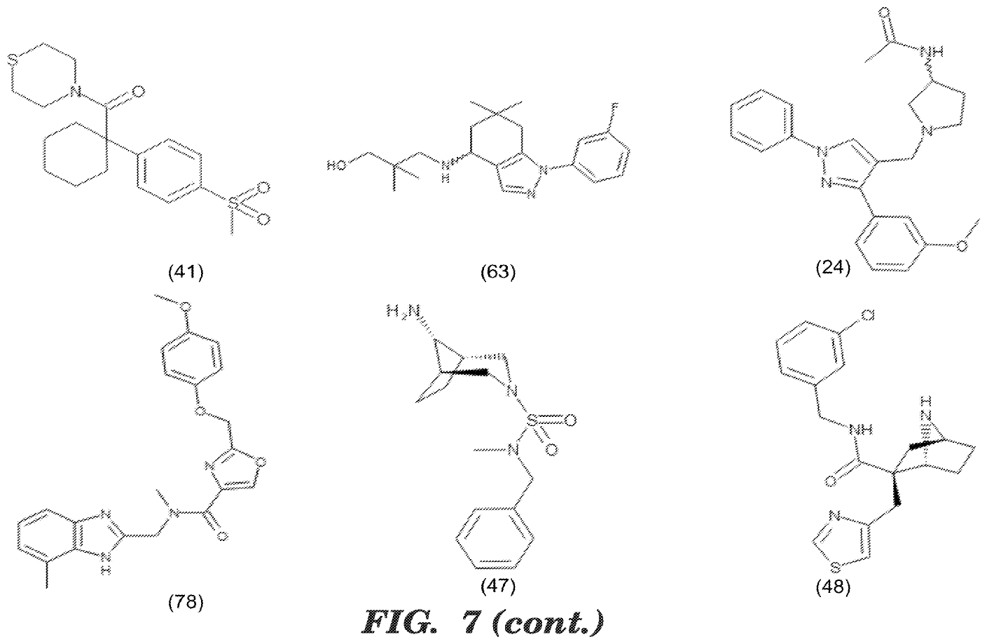

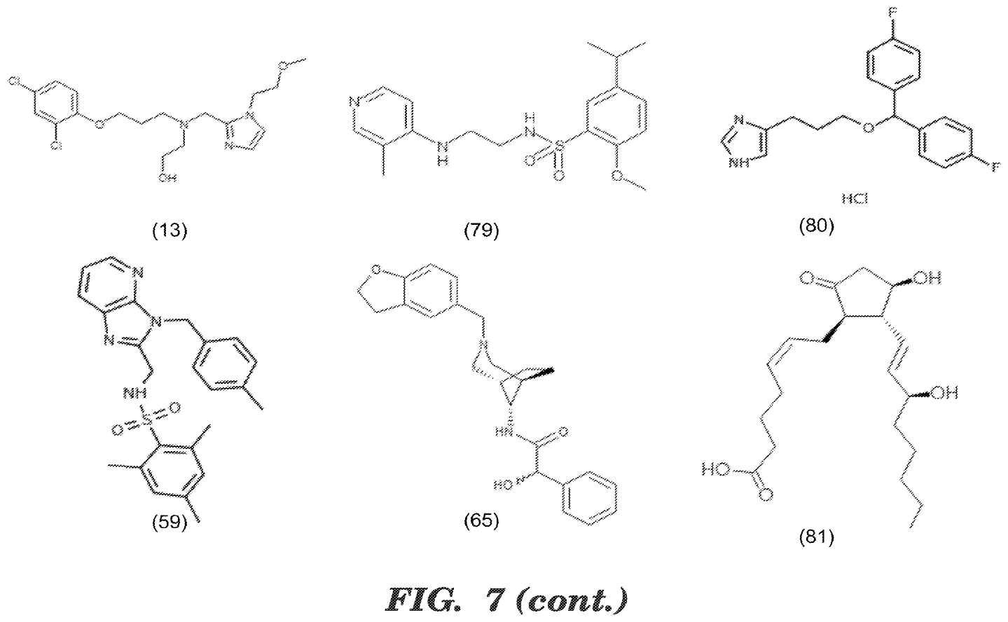

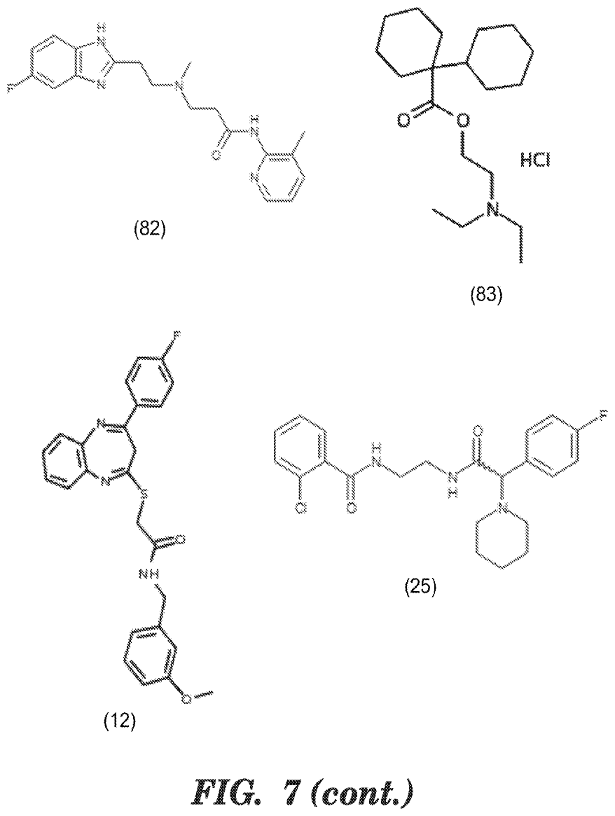

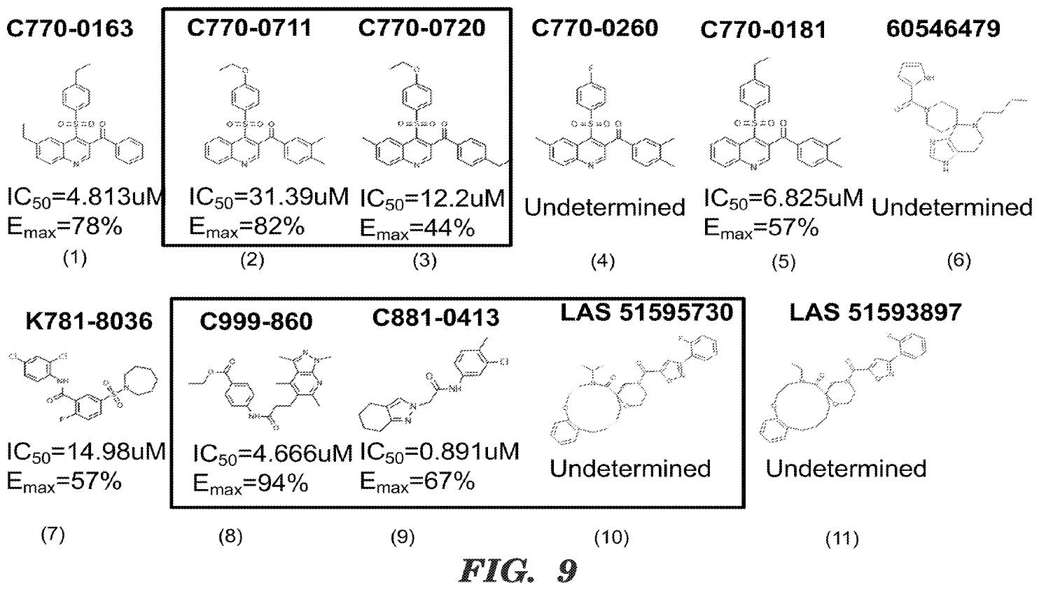

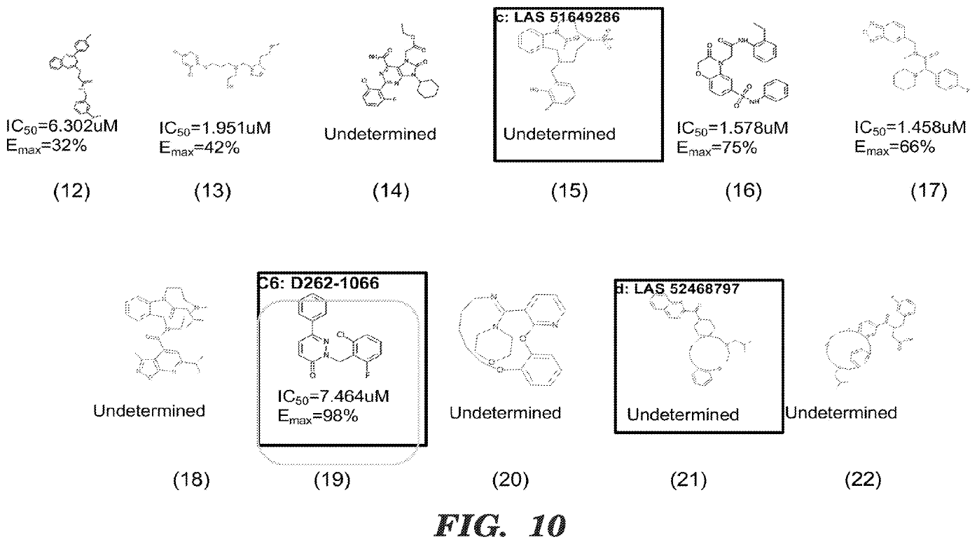

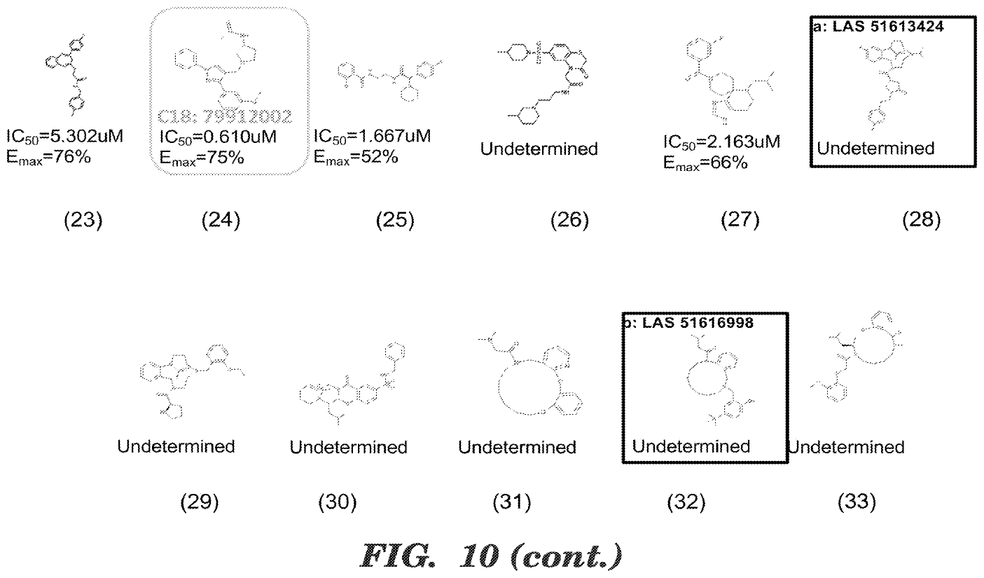

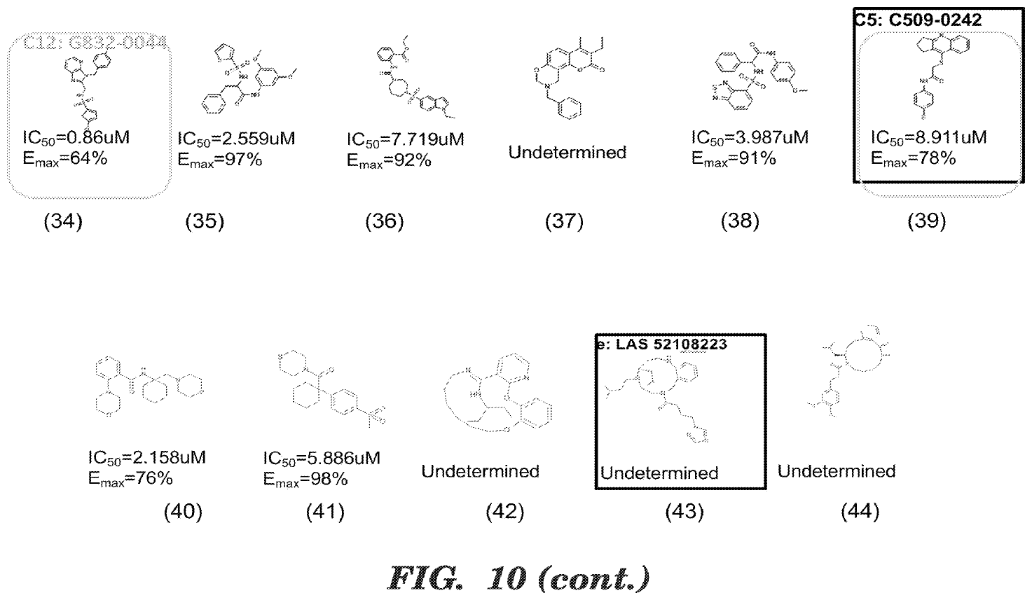

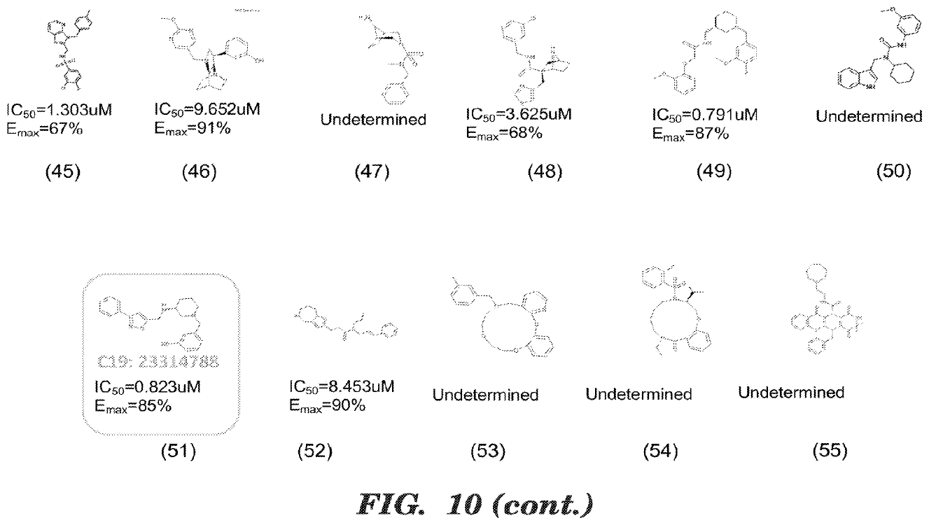

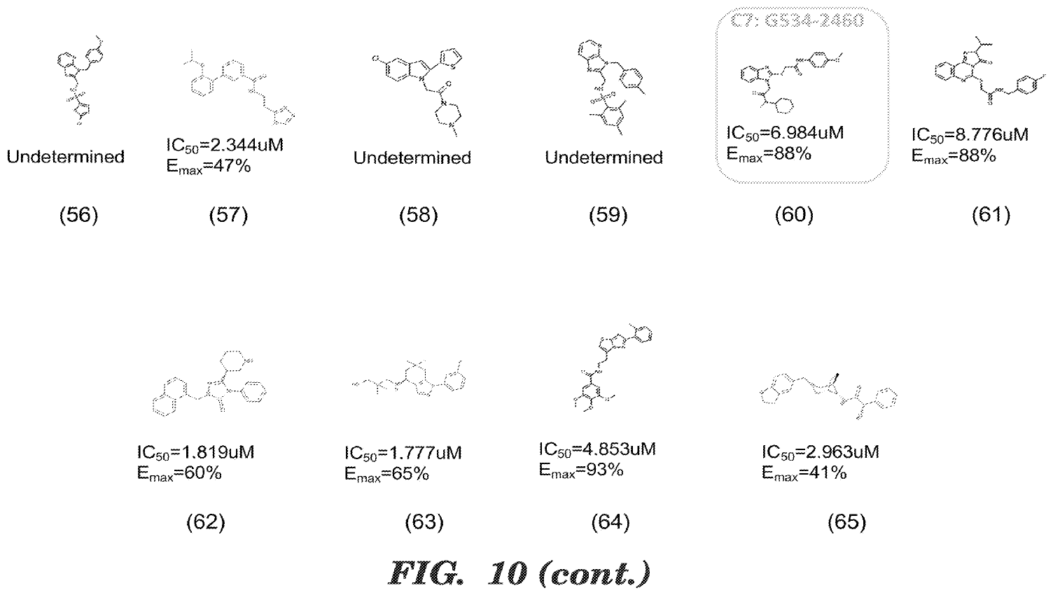

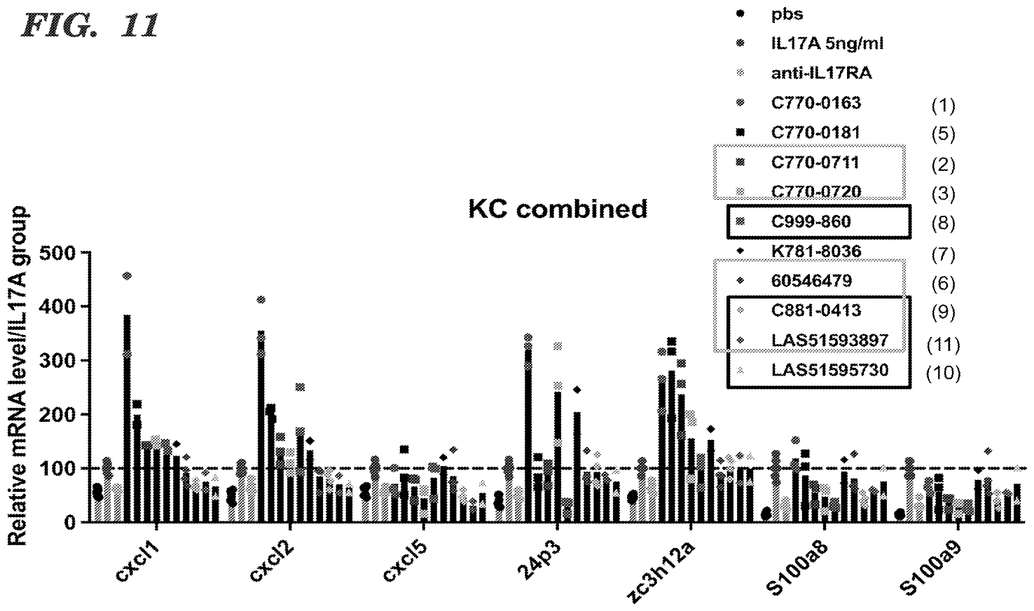

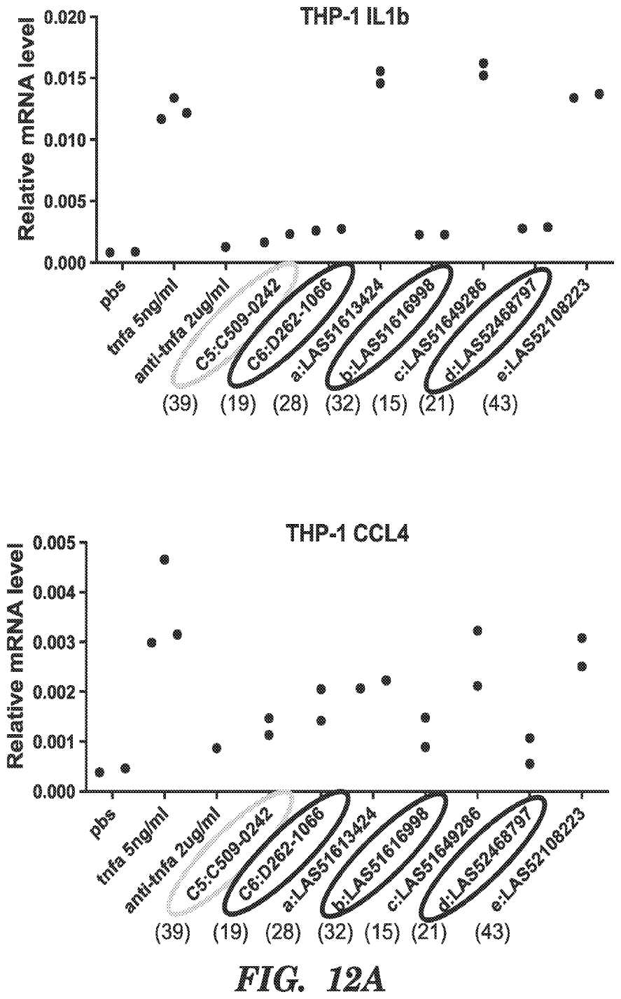

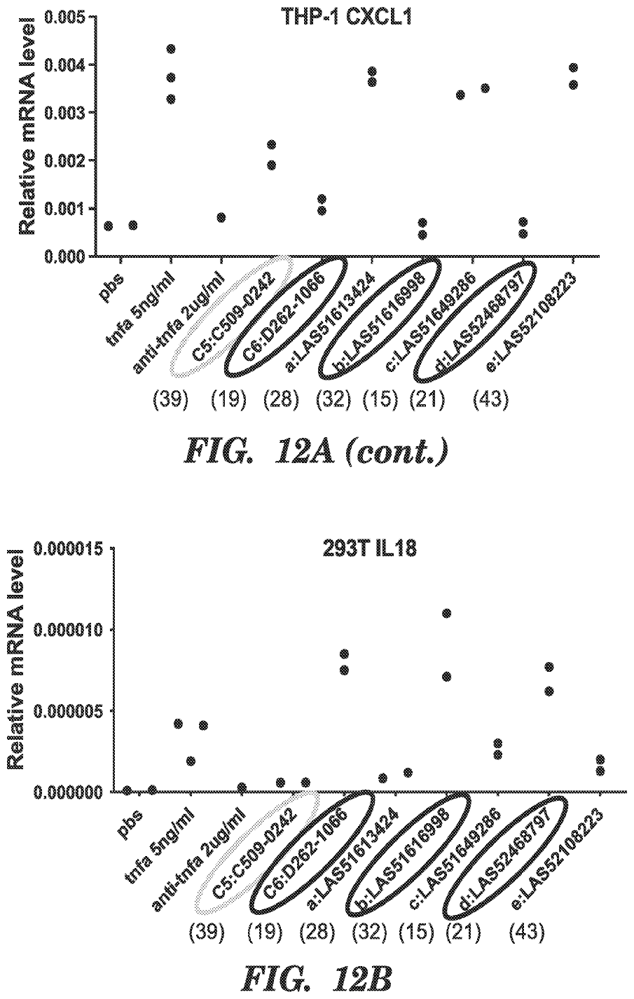

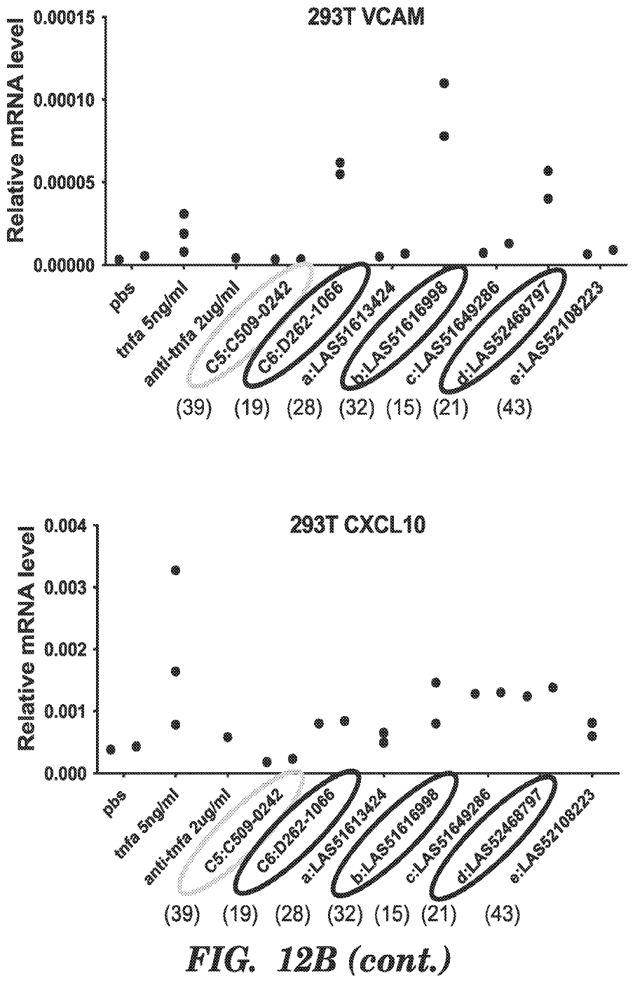

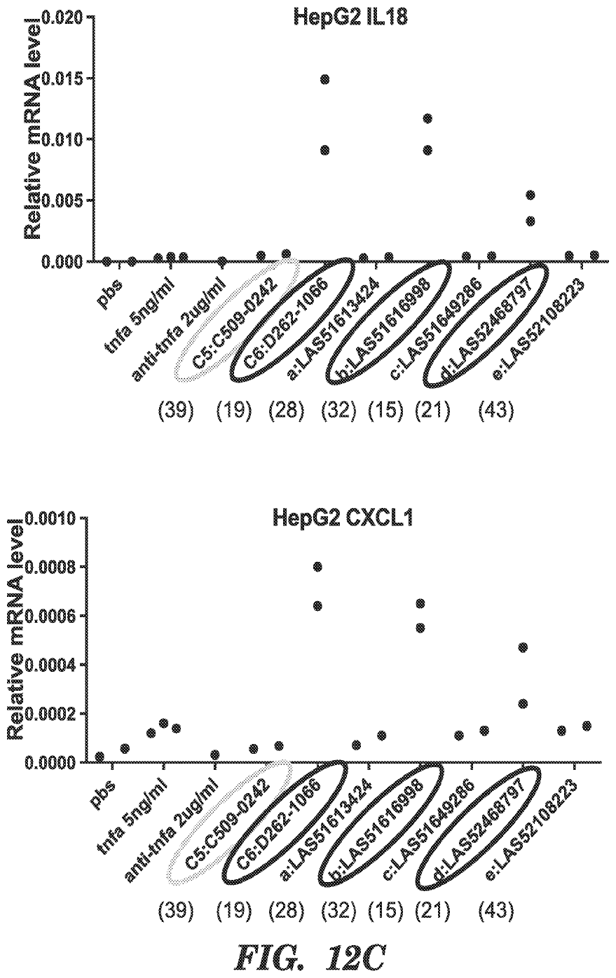

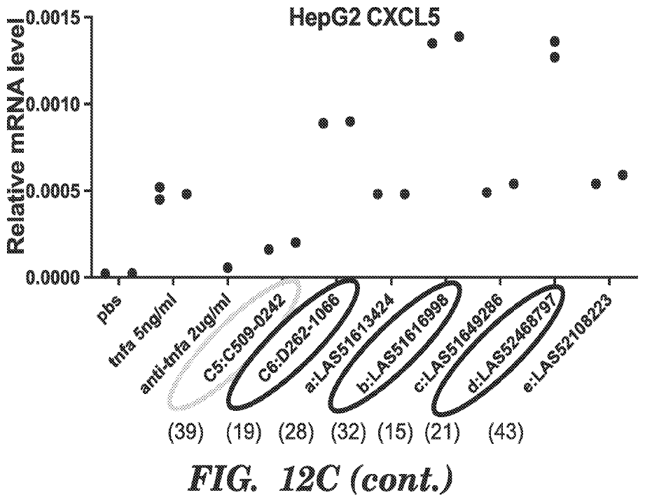

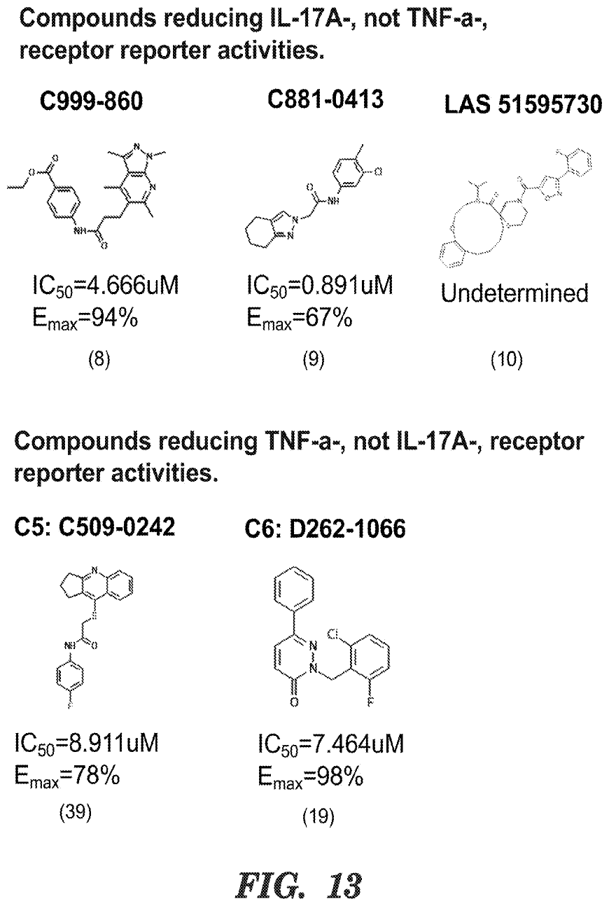

In some embodiments of any of the aspects, the compound is selected from FIG. 2. In some embodiments of any of the aspects, the compound is selected from FIG. 3. In some embodiments of any of the aspects, the compound is selected from FIG. 6. In some embodiments of any of the aspects, the compound is selected from FIG. 7. In some embodiments of any of the aspects, the compound is selected from FIG. 9. In some embodiments of any of the aspects, the compound is selected from FIG. 10. In some embodiments of any of the aspects, the compound is selected from FIG. 11. In some embodiments of any of the aspects, the compound is selected from FIG. 12. In some embodiments of any of the aspects, the compound is selected from FIG. 13.

In some embodiments of any of the aspects, the compound is selected from compounds (1)-(11). In some embodiments of any of the aspects, the compound is selected from compounds (1)-(65). In some embodiments of any of the aspects, the compound is selected from compounds (1)-(83). In some embodiments of any of the aspects, the compound is selected from compounds (12)-(65). In some embodiments of any of the aspects, the compound is selected from compounds (1)-(9) and (66)-(72). In some embodiments of any of the aspects, the compound is selected from compounds (12), (13), (14), (16), (17), (24), (25), (26), (37), (40), (41), (46), (47), (48), (49), (50), (51), (57), (58), (59), (62), (63), (65), (73), (74), (75), (76), (77), (78), (79), (80), (81), (82), and (83).

In some embodiments of any of the aspects, the compound is selected from compounds (2), (3), (6), (8), (9), (10), and (11) (see e.g., FIG. 11). In some embodiments of any of the aspects, the compound is selected from compounds (8), (9), (10), and (11) (see e.g., FIG. 11). In some embodiments of any of the aspects, the compound is selected from compounds (2), (3), (6), (9), and (11) (see e.g., FIG. 11). In some embodiments of any of the aspects, the compound is selected from compounds (2), (3), (8), (9), and (10) (see e.g., FIG. 9, Table 9). In some embodiments of any of the aspects, the compound is compound (2). In some embodiments of any of the aspects, the compound is compound (3). In some embodiments of any of the aspects, the compound is compound (6). In some embodiments of any of the aspects, the compound is compound (8). In some embodiments of any of the aspects, the compound is compound (9). In some embodiments of any of the aspects, the compound is compound (10). In some embodiments of any of the aspects, the compound is compound (11).

In some embodiments of any of the aspects, the compound is selected from compounds (15), (19), (21), (28), (32), (39), and (43) (see e.g., FIG. 10, Table 10). In some embodiments of any of the aspects, the compound is selected from compounds (19), (21), (32), and (39) (see e.g., FIG. 12). In some embodiments of any of the aspects, the compound is compound (15). In some embodiments of any of the aspects, the compound is compound (19). In some embodiments of any of the aspects, the compound is compound (21). In some embodiments of any of the aspects, the compound is compound (28). In some embodiments of any of the aspects, the compound is compound (32). In some embodiments of any of the aspects, the compound is compound (39). In some embodiments of any of the aspects, the compound is compound (43).

In some embodiments of any of the aspects, the compound is selected from compounds (19), (24), (34), (39), (51), and (60) (see e.g., FIG. 10, Table 10). In some embodiments of any of the aspects, the compound is compound (19). In some embodiments of any of the aspects, the compound is compound (24). In some embodiments of any of the aspects, the compound is compound (34). In some embodiments of any of the aspects, the compound is compound (39). In some embodiments of any of the aspects, the compound is compound (51). In some embodiments of any of the aspects, the compound is compound (60).

In some embodiments of any of the aspects, the compound is selected from compounds (8), (9), and (10) (see e.g., FIG. 13). In some embodiments of any of the aspects, the compound is selected from compounds (3), (8), and (9). In some embodiments of any of the aspects, the compound is compound (3). In some embodiments of any of the aspects, the compound is compound (8). In some embodiments of any of the aspects, the compound is compound (9). In some embodiments of any of the aspects, the compound is compound (10). In some embodiments of any of the aspects, the compound is selected from compounds (39) and (19) (see e.g., FIG. 13). In some embodiments of any of the aspects, the compound is compound (19). In some embodiments of any of the aspects, the compound is compound (39).

In some embodiments of any of the aspects, the compound is selected from Formulas (I)-(V). In some embodiments of any of the aspects, the compound is selected from Formulas (I)-(III). In some embodiments of any of the aspects, the compound is Formula (I). In some embodiments of any of the aspects, the compound is Formula (II). In some embodiments of any of the aspects, the compound is Formula (III). In some embodiments of any of the aspects, the compound is selected from Formulas (IV)-(V). In some embodiments of any of the aspects, the compound is Formula (IV). In some embodiments of any of the aspects, the compound is Formula (V).

“Optional” or “optionally” means that the subsequently described circumstance may or may not occur, so that the description includes instances where the circumstance occurs and instances where it does not.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀) aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Non-limiting examples of Ra and R_{b} are each independently hydrogen, alkyl, substituted alkyl, alkenyl, or substituted alkenyl. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (—CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. For example, C₄-C₉ heteroaryl includes heteroaryls that have 4 to 9 carbon atoms in the ring system. Heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2 (1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cycyl are typically used where X and Y indicate the number of carbon atoms in the ring system. For example, C₃-C₈ cyclyl includes cyclyls that have 3 to 8 carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. For example, C₄-C₉ heterocyclyl includes heterocyclyls that have 4-9 carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical-C(O)—. It is noted that the carbonyl radical can be further substituted with a variety of substituents to form different carbonyl groups including acids, acid halides, amides, esters, ketones, and the like.

The term “carboxy” means the radical-C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical-OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Substituents may be protected as necessary and any of the protecting groups commonly used in the art may be employed. Non-limiting examples of protecting groups may be found, for example, in Greene et al., Protective Groups in Organic Synthesis, 3rd Ed. (New York: Wiley, 1999).

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)—O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH (alkyl) or —N(alkyl) (alkyl), respectively, such as —NHCH₃, —N(CH₃)₂, and the like.

The term “mono- or di-alkylaminoalkoxy” means —O-(alkyl)-NH (alkyl) or —O-(alkyl)-N(alkyl) (alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH (alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino”—NH-(alkyl)-(cycloalkyl), such as —NHCH₂-cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH3) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

In various embodiments, compounds of the present invention as disclosed herein may be synthesized using any synthetic method available to one of skill in the art. Non-limiting examples of synthetic methods used to prepare various embodiments of compounds of the present invention are disclosed in the Examples section herein.

### VARIOUS EMBODIMENTS OF THE INVENTION

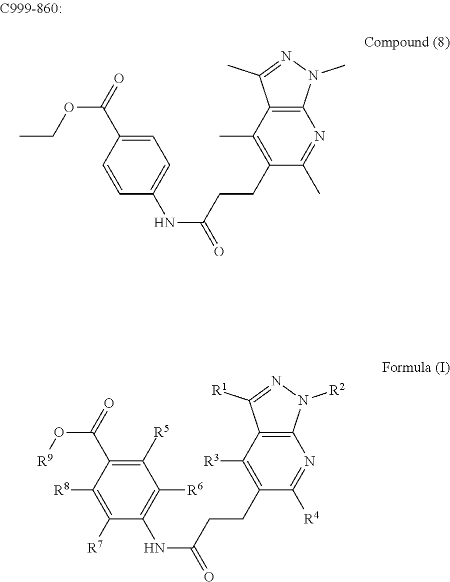

wherein,
R¹ is H, halo, or optionally substituted alkyl;
R² is H or optionally substituted alkyl;
R³ is H, halo, or optionally substituted alkyl;
R⁴ is H, halo, or optionally substituted alkyl;
R⁵ is H, halo, or optionally substituted alkyl;
R⁶ is H, halo, or optionally substituted alkyl;
R⁷ is H, halo, or optionally substituted alkyl;
R⁸ is H, halo, or optionally substituted alkyl; and
R⁹ is H or optionally substituted alkyl.

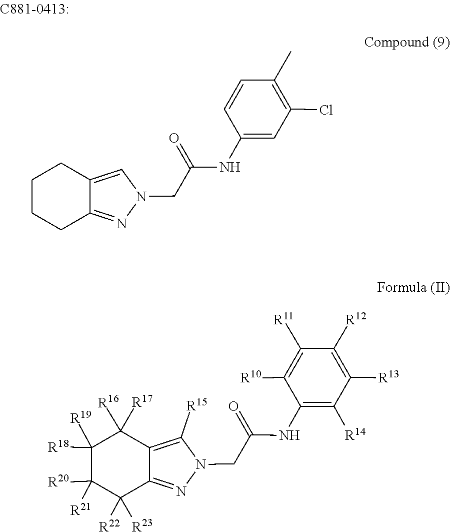

wherein,
R¹⁰ is H, halo, or optionally substituted alkyl;
R¹¹ is H, halo, or optionally substituted alkyl;
R¹² is H, halo, or optionally substituted alkyl;
R¹³ is H, halo, or optionally substituted alkyl;
R¹⁴ is H, halo, or optionally substituted alkyl;
R¹⁵ is H, halo, or optionally substituted alkyl;
R¹⁶ is H, halo, or optionally substituted alkyl;
R¹⁷ is H, halo, or optionally substituted alkyl;
R¹⁸ is H, halo, or optionally substituted alkyl;
R¹⁹ is H, halo, or optionally substituted alkyl;
R²⁰ is H, halo, or optionally substituted alkyl;
R²¹ is H, halo, or optionally substituted alkyl;
R²² is H, halo, or optionally substituted alkyl; and
R²³ is H, halo, or optionally substituted alkyl.

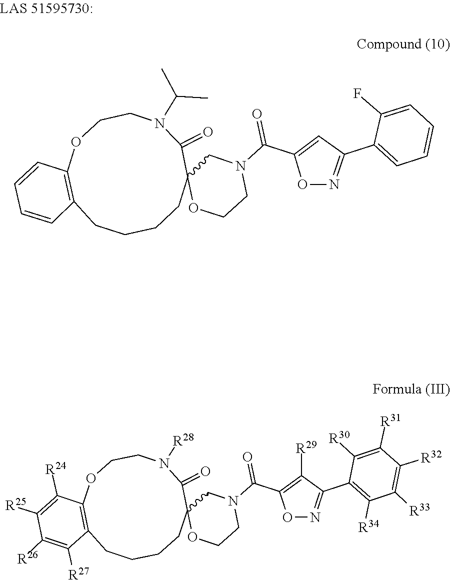

wherein,
R²⁴ is H, halo, or optionally substituted alkyl;
R²⁵ is H, halo, or optionally substituted alkyl;
R²⁶ is H, halo, or optionally substituted alkyl;
R²⁷ is H, halo, or optionally substituted alkyl;
R²⁸ is H or optionally substituted alkyl;
R²⁹ is H, halo, or optionally substituted alkyl;
R³⁰ is H, halo, or optionally substituted alkyl;
R³¹ is H, halo, or optionally substituted alkyl;
R³² is H, halo, or optionally substituted alkyl;
R³³ is H, halo, or optionally substituted alkyl; and
R³⁴ is H, halo, or optionally substituted alkyl.

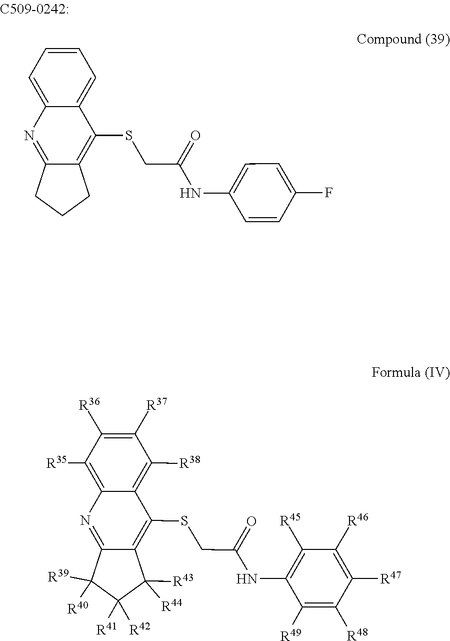

wherein,
R³⁵ is H, halo, or optionally substituted alkyl;
R³⁶ is H, halo, or optionally substituted alkyl;
R³⁷ is H, halo, or optionally substituted alkyl;
R³⁸ is H, halo, or optionally substituted alkyl;
R³⁹ is H, halo, or optionally substituted alkyl;
R⁴⁰ is H, halo, or optionally substituted alkyl;
R⁴¹ is H, halo, or optionally substituted alkyl;
R⁴² is H, halo, or optionally substituted alkyl;
R⁴³ is H, halo, or optionally substituted alkyl;
R⁴⁴ is H, halo, or optionally substituted alkyl;
R⁴⁵ is H, halo, or optionally substituted alkyl;
R⁴⁶ is H, halo, or optionally substituted alkyl;
R⁴⁷ is H, halo, or optionally substituted alkyl;
R⁴⁸ is H, halo, or optionally substituted alkyl; and
R⁴⁹ is H, halo, or optionally substituted alkyl.

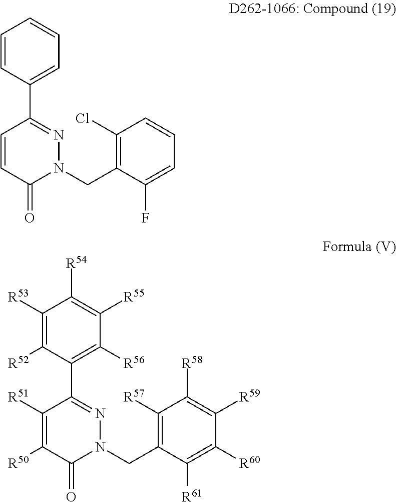

wherein,
R⁵⁰ is H, halo, or optionally substituted alkyl;
R⁵¹ is H, halo, or optionally substituted alkyl;
R⁵² is H, halo, or optionally substituted alkyl;
R⁵³ is H, halo, or optionally substituted alkyl;
R⁵⁴ is H, halo, or optionally substituted alkyl;
R⁵⁵ is H, halo, or optionally substituted alkyl;
R⁵⁶ is H, halo, or optionally substituted alkyl;
R⁵⁷ is H, halo, or optionally substituted alkyl;
R⁵⁸ is H, halo, or optionally substituted alkyl;
R⁵⁹ is H, halo, or optionally substituted alkyl;
R⁶⁰ is H, halo, or optionally substituted alkyl; and
R⁶¹ is H, halo, or optionally substituted alkyl.

Tables 5-10, below, show the IC₅₀ (μM) and Eₘₐₓ of compounds from FIGS. 2, 3, 6 and 7, either ordered from lowest to highest IC₅₀ (see e.g., Tables 1, 3, 5 and 7) or from highest to lowest Eₘₐₓ (see e.g., Tables 2, 4, 6 and 8). As used herein the term IC₅₀ refers to the half-maximal inhibitory concentration, which is a measure of a compound's efficacy. IC₅₀ indicates how much of the compound is needed to inhibit a biological process by half (e.g., IL-17A receptor signaling or TNF-a receptor signaling), thus providing a measure of potency. The lower the value of IC₅₀ for a given compound, the higher the efficacy of the compound. As used herein the term Eₘₐₓ (also referred to as intrinsic activity or efficacy) refers to the maximal effect of the compound at high compound concentrations when all the receptors are occupied by the compound. The higher the value of Eₘₐₓ, the higher the efficacy of the compound. An Eₘₐₓ of 100% is an efficacy equal to the endogenous ligand (e.g., IL-17A or TNF-a). An Eₘₐₓ of between 0% and 100% is an efficacy equal less than the endogenous ligand.

TABLE 1
Compounds reducing IL-17A-receptor reporter activities, ordered
from lowest to highest IC₅₀ (see e.g., FIG. 2, FIG. 9, Table 9)

| Compound # | IC₅₀ (μM) | Eₘₐₓ |
|---|---|---|
| 9 | 0.891 | 67% |
| 8 | 4.666 | 94% |
| 1 | 4.813 | 78% |
| 5 | 6.825 | 57% |
| 3 | 12.2 | 44% |
| 7 | 14.98 | 57% |
| 2 | 31.39 | 82% |
| 4 | Undetermined | Undetermined |
| 6 | Undetermined | Undetermined |
| 10 | Undetermined | Undetermined |
| 11 | Undetermined | Undetermined |

TABLE 2
Compounds reducing IL-17A-receptor reporter activities, ordered
from highest to lowest Eₘₐₓ (see e.g., FIG. 2, FIG. 9, Table 9)

| Compound # | IC₅₀ (μM) | Eₘₐₓ |
|---|---|---|
| 8 | 4.666 | 94% |
| 2 | 31.39 | 82% |
| 1 | 4.813 | 78% |
| 9 | 0.891 | 67% |
| 5 | 6.825 | 57% |
| 7 | 14.98 | 57% |
| 3 | 12.2 | 44% |
| 4 | Undetermined | Undetermined |
| 6 | Undetermined | Undetermined |
| 10 | Undetermined | Undetermined |
| 11 | Undetermined | Undetermined |

TABLE 3
Compounds reducing TNF-a-receptor reporter activities, ordered from
lowest to highest IC₅₀ (see e.g., FIG. 3, FIG. 10, Table 10)

| Compound # | IC₅₀ (μM) | Eₘₐₓ |
|---|---|---|
| 24 | 0.610 | 75% |
| 49 | 0.791 | 87% |
| 51 | 0.823 | 85% |
| 34 | 0.860 | 64% |
| 45 | 1.303 | 67% |
| 17 | 1.458 | 66% |
| 16 | 1.578 | 75% |
| 25 | 1.667 | 52% |
| 63 | 1.777 | 65% |
| 62 | 1.819 | 60% |
| 13 | 1.951 | 42% |
| 40 | 2.158 | 76% |
| 27 | 2.163 | 66% |
| 57 | 2.344 | 47% |
| 35 | 2.559 | 97% |
| 65 | 2.963 | 41% |
| 48 | 3.625 | 68% |
| 38 | 3.987 | 91% |
| 64 | 4.853 | 93% |
| 23 | 5.302 | 76% |
| 41 | 5.886 | 98% |
| 12 | 6.302 | 32% |
| 60 | 6.984 | 88% |
| 19 | 7.464 | 98% |
| 36 | 7.719 | 92% |
| 52 | 8.453 | 90% |
| 61 | 8.776 | 88% |
| 39 | 8.911 | 78% |
| 46 | 9.652 | 91% |
| 14 | Undetermined | Undetermined |
| 15 | Undetermined | Undetermined |
| 18 | Undetermined | Undetermined |
| 20 | Undetermined | Undetermined |
| 21 | Undetermined | Undetermined |
| 22 | Undetermined | Undetermined |
| 26 | Undetermined | Undetermined |
| 28 | Undetermined | Undetermined |
| 29 | Undetermined | Undetermined |
| 30 | Undetermined | Undetermined |
| 31 | Undetermined | Undetermined |
| 32 | Undetermined | Undetermined |
| 33 | Undetermined | Undetermined |
| 37 | Undetermined | Undetermined |
| 42 | Undetermined | Undetermined |
| 43 | Undetermined | Undetermined |
| 44 | Undetermined | Undetermined |
| 47 | Undetermined | Undetermined |
| 50 | Undetermined | Undetermined |
| 53 | Undetermined | Undetermined |
| 54 | Undetermined | Undetermined |
| 55 | Undetermined | Undetermined |
| 56 | Undetermined | Undetermined |
| 58 | Undetermined | Undetermined |
| 59 | Undetermined | Undetermined |

TABLE 4
Compounds reducing TNF-a-receptor reporter activities, ordered from
highest to lowest Eₘₐₓ (see e.g., FIG. 3, FIG. 10, Table 10)

| Compound # | IC₅₀ (μM) | Eₘₐₓ |
|---|---|---|
| 41 | 5.886 | 98% |
| 19 | 7.464 | 98% |
| 35 | 2.559 | 97% |
| 64 | 4.853 | 93% |
| 36 | 7.719 | 92% |
| 38 | 3.987 | 91% |
| 46 | 9.652 | 91% |
| 52 | 8.453 | 90% |
| 60 | 6.984 | 88% |
| 61 | 8.776 | 88% |
| 49 | 0.791 | 87% |
| 51 | 0.823 | 85% |
| 39 | 8.911 | 78% |
| 40 | 2.158 | 76% |
| 23 | 5.302 | 76% |
| 24 | 0.610 | 75% |
| 16 | 1.578 | 75% |
| 48 | 3.625 | 68% |
| 45 | 1.303 | 67% |
| 17 | 1.458 | 66% |
| 27 | 2.163 | 66% |
| 63 | 1.777 | 65% |
| 34 | 0.860 | 64% |
| 62 | 1.819 | 60% |
| 25 | 1.667 | 52% |
| 57 | 2.344 | 47% |
| 13 | 1.951 | 42% |
| 65 | 2.963 | 41% |
| 12 | 6.302 | 32% |
| 14 | Undetermined | Undetermined |
| 15 | Undetermined | Undetermined |
| 18 | Undetermined | Undetermined |
| 20 | Undetermined | Undetermined |
| 21 | Undetermined | Undetermined |
| 22 | Undetermined | Undetermined |
| 26 | Undetermined | Undetermined |
| 28 | Undetermined | Undetermined |
| 29 | Undetermined | Undetermined |
| 30 | Undetermined | Undetermined |
| 31 | Undetermined | Undetermined |
| 32 | Undetermined | Undetermined |
| 33 | Undetermined | Undetermined |
| 37 | Undetermined | Undetermined |
| 42 | Undetermined | Undetermined |
| 43 | Undetermined | Undetermined |
| 44 | Undetermined | Undetermined |
| 47 | Undetermined | Undetermined |
| 50 | Undetermined | Undetermined |
| 53 | Undetermined | Undetermined |
| 54 | Undetermined | Undetermined |
| 55 | Undetermined | Undetermined |
| 56 | Undetermined | Undetermined |
| 58 | Undetermined | Undetermined |
| 59 | Undetermined | Undetermined |

TABLE 5
Compounds reducing IL-17A-receptor reporter activities, ordered
from lowest to highest IC₅₀ (see e.g., FIG. 6, Table 11)

| Compound # | IC₅₀ (μM) | Eₘₐₓ |
|---|---|---|
| 9 | 0.891 | 67% |
| 8 | 4.666 | 94% |
| 1 | 4.813 | 78% |
| 5 | 6.825 | 57% |
| 3 | 12.2 | 44% |
| 7 | 14.98 | 57% |
| 2 | 31.39 | 82% |
| 66 | Undetermined | Undetermined |
| 67 | Undetermined | Undetermined |
| 68 | Undetermined | Undetermined |
| 4 | Undetermined | Undetermined |
| 69 | Undetermined | Undetermined |
| 70 | Undetermined | Undetermined |
| 71 | Undetermined | Undetermined |
| 6 | Undetermined | Undetermined |
| 72 | Undetermined | Undetermined |

TABLE 6
Compounds reducing IL-17A-receptor reporter activities, ordered
from highest to lowest Eₘₐₓ (see e.g., FIG. 6, Table 11)

| Compound # | IC₅₀ (μM) | Eₘₐₓ |
|---|---|---|
| 8 | 4.666 | 94% |
| 2 | 31.39 | 82% |
| 1 | 4.813 | 78% |
| 9 | 0.891 | 67% |
| 5 | 6.825 | 57% |
| 7 | 14.98 | 57% |
| 3 | 12.2 | 44% |
| 66 | Undetermined | Undetermined |
| 67 | Undetermined | Undetermined |
| 68 | Undetermined | Undetermined |
| 4 | Undetermined | Undetermined |
| 69 | Undetermined | Undetermined |
| 70 | Undetermined | Undetermined |
| 71 | Undetermined | Undetermined |
| 6 | Undetermined | Undetermined |
| 72 | Undetermined | Undetermined |

TABLE 7
Compounds reducing TNF-a-receptor reporter activities, ordered
from lowest to highest IC50 (see e.g., FIG. 7, Table 12)

| Compound # | IC₅₀ (μM) | Eₘₐₓ |
|---|---|---|
| 24 | 0.610 | 75% |
| 79 | 0.730 | 29% |
| 49 | 0.791 | 87% |
| 51 | 0.823 | 85% |
| 82 | 1.208 | 51% |
| 76 | 1.355 | 64% |
| 17 | 1.458 | 66% |
| 16 | 1.578 | 75% |
| 25 | 1.667 | 52% |
| 63 | 1.777 | 65% |
| 62 | 1.819 | 60% |
| 13 | 1.951 | 42% |
| 40 | 2.158 | 76% |
| 75 | 2.163 | 66% |
| 57 | 2.344 | 47% |
| 65 | 2.963 | 41% |
| 77 | 3.554 | 60% |
| 48 | 3.625 | 68% |
| 41 | 5.886 | 98% |
| 12 | 6.302 | 32% |
| 74 | 8.453 | 90% |
| 46 | 9.652 | 91% |
| 14 | Undetermined | Undetermined |
| 73 | Undetermined | Undetermined |
| 37 | Undetermined | Undetermined |
| 26 | Undetermined | Undetermined |
| 50 | Undetermined | Undetermined |
| 58 | Undetermined | Undetermined |
| 78 | Undetermined | Undetermined |
| 47 | Undetermined | Undetermined |
| 80 | Undetermined | Undetermined |
| 59 | Undetermined | Undetermined |
| 81 | Undetermined | Undetermined |
| 83 | Undetermined | Undetermined |

TABLE 8
Compounds reducing TNF-a-receptor reporter activities, ordered
from highest to lowest Eₘₐₓ (see e.g., FIG. 7, Table 12)

| Compound # | IC₅₀ (μM) | Eₘₐₓ |
|---|---|---|
| 41 | 5.886 | 98% |
| 46 | 9.652 | 91% |
| 74 | 8.453 | 90% |
| 49 | 0.791 | 87% |
| 51 | 0.823 | 85% |
| 40 | 2.158 | 76% |
| 24 | 0.610 | 75% |
| 16 | 1.578 | 75% |
| 48 | 3.625 | 68% |
| 17 | 1.458 | 66% |
| 75 | 2.163 | 66% |
| 63 | 1.777 | 65% |
| 76 | 1.355 | 64% |
| 62 | 1.819 | 60% |
| 77 | 3.554 | 60% |
| 25 | 1.667 | 52% |
| 82 | 1.208 | 51% |
| 57 | 2.344 | 47% |
| 13 | 1.951 | 42% |
| 65 | 2.963 | 41% |
| 12 | 6.302 | 32% |
| 79 | 0.730 | 29% |
| 14 | Undetermined | Undetermined |
| 73 | Undetermined | Undetermined |
| 37 | Undetermined | Undetermined |
| 26 | Undetermined | Undetermined |
| 50 | Undetermined | Undetermined |
| 58 | Undetermined | Undetermined |
| 78 | Undetermined | Undetermined |
| 47 | Undetermined | Undetermined |
| 80 | Undetermined | Undetermined |
| 59 | Undetermined | Undetermined |
| 81 | Undetermined | Undetermined |
| 83 | Undetermined | Undetermined |

In some embodiments of any of the aspects, the compound is selected from compounds (1), (2), (3), (5), (7), (8), and (9) (see e.g., Tables 1, 2 and 9, and FIG. 2). In some embodiments of any of the aspects, the compound is selected from compounds (12), (13), (16), (17), (19), (23), (24), (25), (27), (34), (35), (36), (38), (39), (40), (41), (45), (46), (48), (49), (51), (52), (57), (60), (61), (62), (63), (64), and (65) (see e.g., Tables 3, 4 and 10, and FIG. 3). In some embodiments of any of the aspects, the compound is selected from compounds (1), (2), (3), (5), (7), (8), and (9) (see e.g., Tables 5, 6 and 11, and FIG. 6). In some embodiments of any of the aspects, the compound is selected from compounds (12), (13), (16), (17), (24), (25), (40), (41), (46), (48), (49), (51), (57), (62), (63), (65), (74), (75), (76), (77), (79), and (82) (see e.g., Tables 7, 8 and 12, and FIG. 7).

In some embodiments of any of the aspects, the compound is selected from the first 1, 2, 3, 4, 5, 6, or 7 compounds listed in Table 1 or Table 2. In some embodiments of any of the aspects, the compound is selected from the first 1, 2, 3, 4, 5, 6, 7, 8, 9, 10, 11, 12, 13, 14, 15, 16, 17, 18, 19, 20, 21, 22, 23, 24, 25, 26, 27, 28, or 29 compounds listed in Table 3 or Table 4. In some embodiments of any of the aspects, the compound is selected from the first 1, 2, 3, 4, 5, 6, or 7 compounds listed in Table 5 or Table 6. In some embodiments of any of the aspects, the compound is selected from the first 1, 2, 3, 4, 5, 6, 7, 8, 9, 10, 11, 12, 13, 14, 15, 16, 17, 18, 19, 20, 21, or 22 compounds listed in Table 7 or Table 8.

In some embodiments of any of the aspects, the compound is selected from compounds (1), (3), (5), (8), and (9) (see e.g., Tables 1 and 9, FIG. 2). In some embodiments of any of the aspects, the compound is selected from compounds (1), (2), (5), (8), and (9) (see e.g., Tables 2 and 9, FIG. 2). In some embodiments of any of the aspects, the compound is selected from compounds (24), (34), (45), (49), and (51) (see e.g., Tables 3 and 10, FIG. 3). In some embodiments of any of the aspects, the compound is selected from compounds (19), (35), (36), (41), and (64) (see e.g., Tables 4 and 10, FIG. 3). In some embodiments of any of the aspects, the compound is selected from compounds (1), (3), (5), (8), and (9) (see e.g., Tables 5 and 11, FIG. 6). In some embodiments of any of the aspects, the compound is selected from compounds (1), (2), (5), (8), and (9) (see e.g., Tables 6 and 11, FIG. 6). In some embodiments of any of the aspects, the compound is selected from compounds (24), (49), (51), (79), and (82) (see e.g., Tables 7 and 12, FIG. 7). In some embodiments of any of the aspects, the compound is selected from compounds (41), (46), (49), (51), and (74).

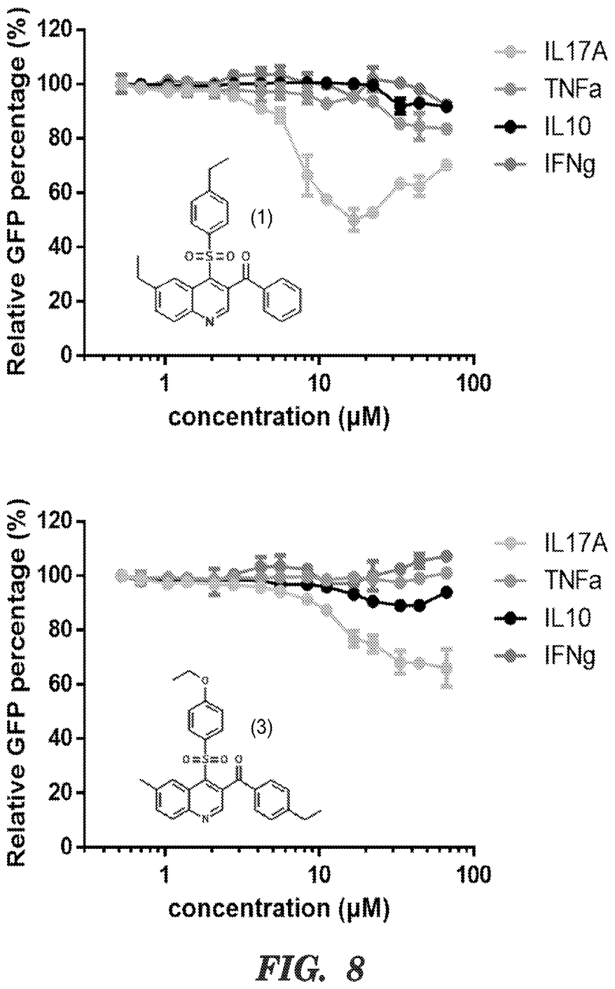

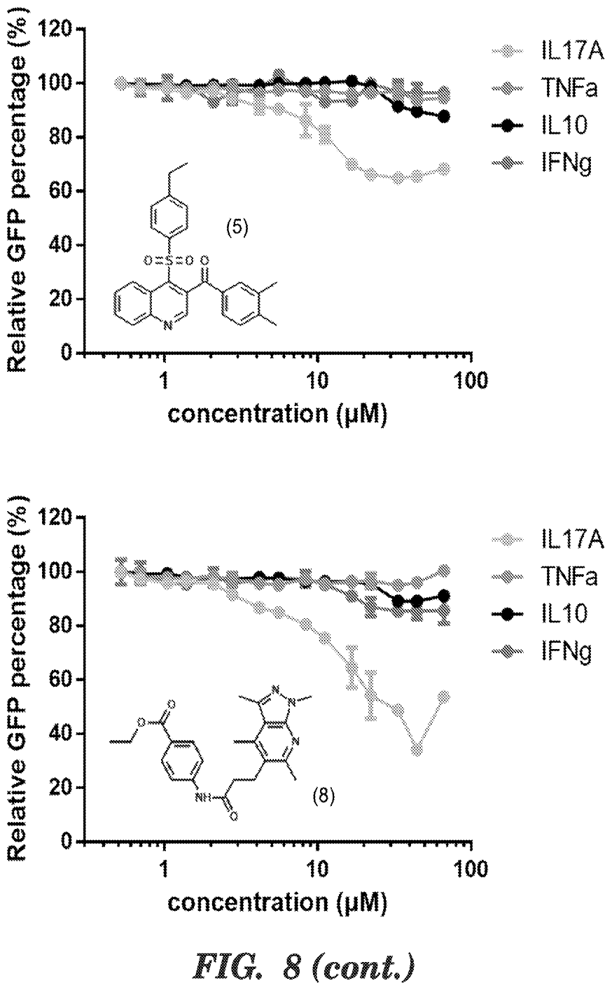

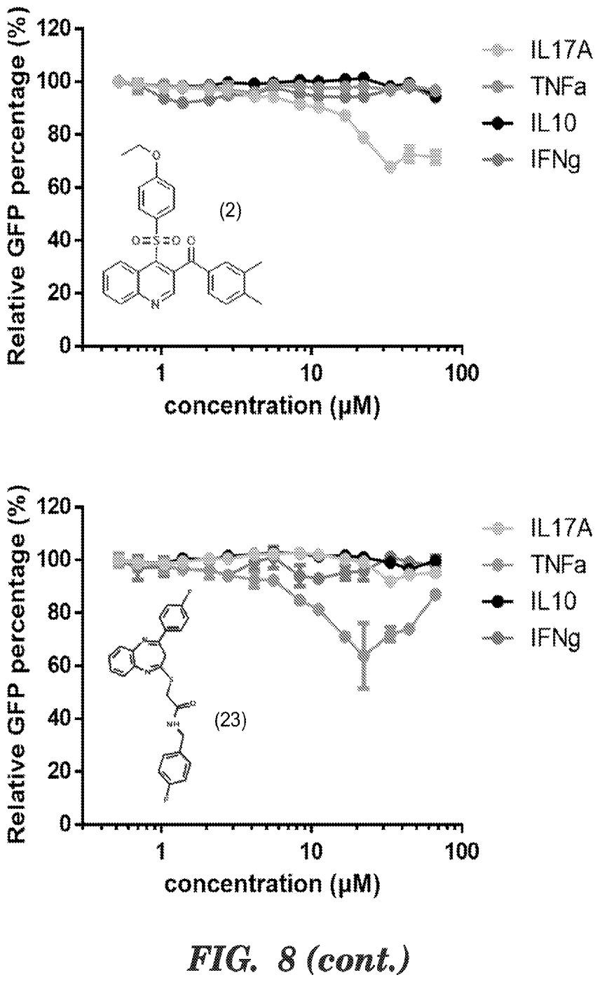

In some embodiments of any of the aspects, the compound is selected from compounds (1), (2), (3), (5), (8), and (23) (see e.g., FIG. 8). In some embodiments of any of the aspects, the compound is selected from compounds (1), (2), (3), (5), and (8). In some embodiments of any of the aspects, the compound is compound (1). In some embodiments of any of the aspects, the compound is compound (2). In some embodiments of any of the aspects, the compound is compound (3). In some embodiments of any of the aspects, the compound is compound (5). In some embodiments of any of the aspects, the compound is compound (8). In some embodiments of any of the aspects, the compound is compound (23).

In some embodiments of any of the aspects, the compound(s) (e.g., at least one of compounds (1)-(83)) present in a composition, or combination, of the disclosure exhibit an increased utility that is not exhibited when said compound(s) occur alone or when said compound(s) are present at a naturally occurring concentration. In some embodiments of any of the aspects, compositions of the disclosure, comprising compound(s) as taught herein, exhibit a synergistic effect on imparting at least one improved trait in an IL-17a-associated disease or disorder, an IL-17f-associated disease or disorder, or a TNF-α-associated disease or disorder.

In some embodiments of any of the aspects, the compositions of the disclosure—comprising compound(s) (e.g., at least one of compounds (1)-(83)) as taught herein—exhibit markedly different characteristics/properties compared to their closest naturally occurring counterpart. That is, the compositions of the disclosure exhibit markedly different functional and/or structural characteristics/properties, as compared to their closest naturally occurring counterpart. For instance, the compound(s) of the disclosure are structurally different from a compound as it naturally exists, for at least the following reasons: said compound can be isolated and purified; said compound can be present at concentrations that do not occur naturally; said compound can be associated with acceptable carriers that do not occur naturally; said compound can be formulated to be shelf-stable and exist outside the natural environment; and/or said compound can be combined with other compound(s) or carrier(s) at concentrations that do not exist naturally.

Further, the compound(s) (e.g., at least one of compounds (1)-(83)) of the disclosure are functionally different from a compound as it naturally exists, for at least the following reasons: said compound when applied in an isolated and purified form can lead to treatment of an IL-17a-associated disease or disorder, an IL-17f-associated disease or disorder, or a TNF-α-associated disease or disorder; said compound can be formulated to be shelf-stable and able to exist outside the natural environment, such that the compound has a utility as a supplement capable of administration to a subject, wherein the compound could not have such a utility in its natural state, as the compound would be unable to survive without the intervention of the hand of man to formulate the compound into a shelf-stable state and impart this utility that has the aforementioned functional characteristics not possessed by the compound in its natural state of existence.

### Treatment Methods

The compounds or compositions described herein can be administered to a subject in need thereof, for instance for the treatment of an IL-17a-associated disease or disorder, an IL-17f-associated disease or disorder, or a TNF-α-associated disease or disorder. In some embodiments, the method of treatment can comprise first diagnosing a subject or patient who can benefit from treatment by a composition described herein. In some embodiments, such diagnosis comprises detecting or measuring an increased (or decreased) level of IL-17a, IL-17f, or TNF-α in a sample from the subject or patient compared to a control, each of which are examples of an abnormal level of each analyte. In some embodiments, the method further comprises administering to the patient a compound or composition as described herein.

In some embodiments, the subject has previously been determined to have an abnormal level of an analyte described herein relative to a reference. In some embodiments, the reference level can be the level in a sample of similar cell type, sample type, sample processing, and/or obtained from a subject of similar age, sex and other demographic parameters as the sample/subject. In some embodiments, the test sample and control reference sample are of the same type, that is, obtained from the same biological source, and comprising the same composition, e.g., the same number and type of cells.

The term “sample” or “test sample” as used herein denotes a sample taken or isolated from a biological organism, e.g., a blood or plasma sample from a subject. In some embodiments of any of the aspects, the technology described herein encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the step of determining if the subject has an abnormal level of an analyte described herein can comprise i) obtaining or having obtained a sample from the subject and ii) performing or having performed an assay on the sample obtained from the subject to determine/measure the level of the analyte in the subject. In some embodiments of any of the aspects, the step of determining if the subject has an abnormal level of an analyte described herein can comprise performing or having performed an assay on a sample obtained from the subject to determine/measure the level of analyte in the subject. In some embodiments of any of the aspects, the step of determining if the subject has an abnormal level of an analyte described herein can comprise ordering or requesting an assay on a sample obtained from the subject to determine/measure the level of the analyte in the subject. In some embodiments of any of the aspects, the step of determining if the subject has an abnormal level of an analyte described herein can comprise receiving the results of an assay on a sample obtained from the subject to determine/measure the level of the analyte in the subject. In some embodiments of any of the aspects, the step of determining if the subject has an abnormal level of an analyte described herein can comprise receiving a report, results, or other means of identifying the subject as a subject with a decreased level of the analyte.

In one aspect of any of the embodiments, described herein is a method of treating an IL-17a-associated disease or disorder, an IL-17f-associated disease or disorder, or a TNF-α-associated disease or disorder in a subject in need thereof, the method comprising: a) determining if the subject has an abnormal level of an analyte described herein; and b) instructing or directing that the subject be administered a composition comprising at least one of compounds (1)-(83) as described herein if the level of the analyte is decreased relative to a reference. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

### Administration

In some embodiments, the methods described herein relate to treating a subject having or diagnosed as having an IL-17a-associated disease or disorder, an IL-17f-associated disease or disorder, or a TNF-α-associated disease or disorder. Subjects having an IL-17a-associated disease or disorder, an IL-17f-associated disease or disorder, or a TNF-α-associated disease or disorder can be identified by a physician using methods of measuring IL-17a, IL-17f, or TNF-α, or by current methods of diagnosing an IL-17a-associated disease or disorder, an IL-17f-associated disease or disorder, or a TNF-α-associated disease or disorder. Symptoms and/or complications of an IL-17a-associated disease or disorder, an IL-17f-associated disease or disorder, or a TNF-α-associated disease or disorder which characterize these conditions and aid in diagnosis are well known in the art. A family history of an IL-17a-associated disease or disorder, an IL-17f-associated disease or disorder, or a TNF-α-associated disease or disorder, or exposure to risk factors for an IL-17a-associated disease or disorder, an IL-17f-associated disease or disorder, or a TNF-α-associated disease or disorder can also aid in determining if a subject is likely to have an IL-17a-associated disease or disorder, an IL-17f-associated disease or disorder, or a TNF-α-associated disease or disorder or in making a diagnosis of an IL-17a-associated disease or disorder, an IL-17f-associated disease or disorder, or a TNF-α-associated disease or disorder.

The compositions and methods described herein can be administered to a subject having or diagnosed as having an IL-17a-associated disease or disorder, an IL-17f-associated disease or disorder, or a TNF-α-associated disease or disorder. In some embodiments, the methods described herein comprise administering an effective amount of compositions described herein, e.g., at least one of compounds (1)-(83) to a subject in order to alleviate a symptom of an IL-17a-associated disease or disorder, an IL-17f-associated disease or disorder, or a TNF-α-associated disease or disorder. As used herein, “alleviating a symptom of an IL-17a-associated disease or disorder, an IL-17f-associated disease or disorder, or a TNF-α-associated disease or disorder” is ameliorating any condition or symptom associated with the IL-17a-associated disease or disorder, the IL-17f-associated disease or disorder, or the TNF-α-associated disease or disorder. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of composition or compound needed to alleviate at least one or more symptom of the disease or disorder, and relates to a sufficient amount of pharmacological composition to provide the desired effect. The term “therapeutically effective amount” therefore refers to an amount of composition or compound that is sufficient to provide a particular modulated IL-17a/IL-17 receptor-induced signalling effect, modulated IL-17f/IL-17 receptor-induced signalling effect, or modulated TNF-α/TNF receptor-induced signalling effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of compound or composition, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for levels of IL-17a, IL-17f, or TNF-α, among others. The dosage can be determined by a physician and adjusted, as necessary, to suit observed effects of the treatment.

In some embodiments, the technology described herein relates to a pharmaceutical composition comprising at least one compound (e.g., at least one of compounds (1)-(83)) as described herein, and optionally a pharmaceutically acceptable carrier. In some embodiments, the active ingredients of the pharmaceutical composition comprise at least one compound (e.g., at least one of compounds (1)-(83)) as described herein. In some embodiments, the active ingredients of the pharmaceutical composition consist essentially of at least one compound (e.g., at least one of compounds (1)-(83)) as described herein. In some embodiments, the active ingredients of the pharmaceutical composition consist of at least one compound (e.g., at least one of compounds (1)-(83)) as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. at least one compound (e.g., at least one of compounds (1)-(83)) as described herein.

In some embodiments, the pharmaceutical composition comprising at least one compound (e.g., at least one of compounds (1)-(83)) as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of at least one compound (e.g., at least one of compounds (1)-(83)) as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of at least one compound (e.g., at least one of compounds (1)-(83)) as disclosed herein can also be incorporated into the parenteral dosage forms of the disclosure, including conventional and controlled-release parenteral dosage forms.

Pharmaceutical compositions comprising at least one compound (e.g., at least one of compounds (1)-(83)) can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the at least one compound (e.g., at least one of compounds (1)-(83)) can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropyl methylcellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the at least one compound (e.g., at least one of compounds (1)-(83)) described herein is administered as a monotherapy, e.g., another treatment for the IL-17a-associated disease or disorder, the IL-17f-associated disease or disorder, or the TNF-α-associated disease or disorder is not administered to the subject.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs-such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising the at least one compound (e.g., at least one of compounds (1)-(83)) as described herein can be administered to a patient once. In certain embodiments, an effective dose of a composition comprising the at least one compound (e.g., at least one of compounds (1)-(83)) can be administered to a patient repeatedly. For systemic administration, subjects can be administered a therapeutic amount of a composition comprising the at least one compound (e.g., at least one of compounds (1)-(83)), such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g., an IL-17a-associated disease or disorder, an IL-17f-associated disease or disorder, or a TNF-α-associated disease or disorder, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to compound or composition. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising at least one compound (e.g., at least one of compounds (1)-(83)) can be administered over a period of time, such as over a 5 minute, 10 minute, 15 minute, 20 minute, or 25 minute period.

The dosage ranges for the administration of the compound(s), according to the methods described herein depend upon, for example, the form of each compound, its potency, and the extent to which symptoms, markers, or indicators of a condition described herein are desired to be reduced, for example the percentage reduction desired for an IL-17a-associated disease or disorder, an IL-17f-associated disease or disorder, or a TNF-α-associated disease or disorder. The dosage should not be so large as to cause adverse side effects, such as autoimmunity or inflammation. Generally, the dosage will vary with the age, condition, and sex of the patient and can be determined by one of skill in the art. The dosage can also be adjusted by the individual physician in the event of any complication.

The efficacy of the compounds or compositions described herein in, e.g. the treatment of a condition described herein, or to induce a response as described herein (e.g., an IL-17a-associated disease or disorder, an IL-17f-associated disease or disorder, or a TNF-α-associated disease or disorder can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., the level of IL-17a, IL-17f, or TNF-α. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of an IL-17a-associated disease or disorder, an IL-17f-associated disease or disorder, or a TNF-α-associated disease or disorder. When using an experimental animal model, efficacy of treatment is evidenced when a statistically significant change in a marker is observed, e.g., IL-17a, IL-17f, or TNF-α, or signaling components down-stream of IL-17a, IL-17f, or TNF-α. In vitro and animal model assays can allow the assessment of a given dose of a compound or composition described herein.

### Autoimmune Diseases

In various embodiments, at least one compound described herein (e.g., at least one of compounds (1)-(83)) can be used to treat autoimmune disease. “Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Autoantigens, as used herein, are endogenous proteins or fragments thereof that elicit this pathogenic immune response. Autoantigen can be any substance or a portion thereof normally found within a mammal that, in an autoimmune disease, becomes the primary (or a primary) target of attack by the immune system. The term also includes antigenic substances that induce conditions having the characteristics of an autoimmune disease when administered to mammals. Additionally, the term includes peptic subclasses consisting essentially of immunodominant epitopes or immunodominant epitope regions of autoantigens. Immunodominant epitopes or regions in induced autoimmune conditions are fragments of an autoantigen that can be used instead of the entire autoantigen to induce the disease. In humans afflicted with an autoimmune disease, immunodominant epitopes or regions are fragments of antigens specific to the tissue or organ under autoimmune attack and recognized by a substantial percentage (e.g. a majority though not necessarily an absolute majority) of autoimmune attack T-cells.

Autoantigens that are known to be associated with autoimmune disease include myelin proteins with demyelinating diseases, e.g. multiple sclerosis and experimental autoimmune myelitis; collagens and rheumatoid arthritis; insulin, proinsulin, glutamic acid decarboxylase 65 (GAD65); islet cell antigen (ICA512; ICA12) with insulin dependent diabetes.

A common feature in a number of autoimmune related diseases and inflammatory conditions is the involvement of pro-inflammatory CD4+ T cells. These T cells are responsible for the release of inflammatory, Th1 type cytokines. Cytokines characterized as Th1 type include interleukin 2 (IL-2), γ-interferon, TNFα and IL-12. Such pro-inflammatory cytokines act to stimulate the immune response, in many cases resulting in the destruction of autologous tissue. Cytokines associated with suppression of T cell response are the Th2 type, and include IL-10, IL-4 and TGF-β. It has been found that Th1 and Th2 type T cells may use the identical antigen receptor in response to an immunogen; in the former producing a stimulatory response and in the latter a suppressive response.

Provided herein is a method of treating an autoimmune disease, which comprises administering an effective amount of a compound or composition as described herein to a patient in need thereof. In one embodiment of any one of the methods described, the autoimmune disorder is selected from the group consisting of thyroiditis, type 1 diabetes mellitus, Hashimoto's thyroidits, Graves' disease, celiac disease, multiple sclerosis, Guillain-Barre syndrome, Addison's disease, and Raynaud's phenomenon, Goodpasture's disease, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, guttate psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritus scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, erythema multiforme, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, an immune complex disorder such as immune complex nephritis, antibody-mediated nephritis, polyneuropathies, chronic neuropathy such as IgM polyneuropathies or IgM-mediated neuropathy, and autoimmune or immune-mediated thrombocytopenia such as idiopathic thrombocytopenia purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, lupoid hepatitis, giant-cell hepatitis, autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amyotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies including channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, Sampter's syndrome, Caplan's syndrome, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic fasciitis, Shulman's syndrome, Felty's syndrome, cyclitis such as chronic cyclitis, heterochromic cyclitis, iridocyclitis (acute or chronic), or Fuch's cyclitis, Henoch-Schonlein purpura, SCID, sepsis, endotoxemia, post-vaccination syndromes, Evan's syndrome, autoimmune gonadal failure, Sydenham's chorea, post-streptococcal nephritis, thromboangiitis obliterans, thyrotoxicosis, tabes dorsalis, chorioiditis, giant-cell polymyalgia, chronic hypersensitivity pneumonitis, keratoconjunctivitis sicca, idiopathic nephritic syndrome, minimal change nephropathy, benign familial and ischemia-reperfusion injury, transplant organ reperfusion, retinal autoimmunity, aphthae, aphthous stomatitis, arteriosclerotic disorders, aspermiogenesis, autoimmune hemolysis, Boeck's disease, enteritis allergica, erythema nodosum leprosum, idiopathic facial paralysis, chronic fatigue syndrome, febris rheumatica, Hamman-Rich's disease, sensoneural hearing loss, ileitis regionalis, leucopenia, transverse myelitis, primary idiopathic myxedema, ophthalmia symphatica, polyradiculitis acuta, pyoderma gangrenosum, acquired splenic atrophy, vitiligo, toxic-shock syndrome, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndrome type I, adult-onset idiopathic hypoparathyroidism (AOIH), myocarditis, nephrotic syndrome, primary sclerosing cholangitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, granulomas containing eosinophils, seronegative spondyloarthritides, polyendocrine autoimmune disease, sclerosing cholangitis, sclera, episclera, Bruton's syndrome, transient hypogammaglobulinemia of infancy, Wiskott-Aldrich syndrome, ataxia telangiectasia syndrome, angiectasis, autoimmune disorders associated with collagen disease, rheumatism, allergic hypersensitivity disorders, glomerulonephritides, reperfusion injury, ischemic re-perfusion disorder, lymphomatous tracheobronchitis, inflammatory dermatoses, dermatoses with acute inflammatory components, and autoimmune uveoretinitis (AUR).

Autoimmune diseases can be mediated by IgG, by inappropriately high levels of IgG, auto-reactive IgG, and or immune complex. Non-limiting examples of IgG-mediated autoimmune diseases include Kawasaki disease, Sjogren's disease, Guillain-Barré, inflammatory bowel disease (IBD), Crohn's disease, ulcerative colitis, systemic lupus erythematosus (SLE), lupus arthritis, lupus nephritis, idiopathic thrombocytopenia purpura, rheumatoid arthritis (RA), warm autoimmune hemolytic anemia, heparin induced thrombocytopenia, thrombotic thrombocytopenia purpura, IgA nephritis, pemphigus vulgaris, systemic sclerosis, Wegener's granulomatosis/granulomatosis with polyangiitis, myasthenia gravis, Addison's disease, ankylosing spondylitis, Behçet's syndrome, celiac disease, Goodpasture syndrome/anti-glomerular basement membrane disease, idiopathic membranous glomerulonephritis, Hashimoto's disease, autoimmune pancreatitis, autoimmune hepatitis, primary biliary sclerosis, multiple sclerosis, vasculitis, psoriasis vulgaris, sarcoidosis, type 1 diabetes gestational alloimmune liver disease, Rh disease, ABO incompatibility, neonatal lupus, hemolytic disease of the newborn, neonatal alloimmune thrombocytopenia, neonatal alloimmune neutropenia, and/or neonatal myasthenia gravis.

In some embodiments, “immune complex” and “immunocomplexed antibody” are used interchangeably. In some embodiments, the immune complex is an immune complex of antigen+antigen-specific antibody. In some embodiments and particularly in some assays as described herein, the immune complex is artificial, i.e., does not occur naturally in the mammal. For example, the immune complex may be a multimeric complex of 4-hydroxy-5-iodo-3-nitrophenyl acetic acid (NIP), chicken ovalbumin (OVA), and an anti-NIP antibody. In this context, the anti-NIP antibody is a chimeric IgG antibody that contains a murine variable region specific for 4-hydroxy-5-iodo-3-nitrophenyl acetic acid and an Fc domain from wild-type human IgG1 (see e.g., Claypool, 2004, Mol. Biol. Cell 15:1746-1759).

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of an IL-17a-associated disease or disorder, an IL-17f-associated disease or disorder, or a TNF-α-associated disease or disorder. A subject can be male or female.

A subject can be one who has been previously diagnosed with or identified as suffering from or having a condition in need of treatment (e.g. an IL-17a-associated disease or disorder, an IL-17f-associated disease or disorder, or a TNF-α-associated disease or disorder) or one or more complications related to such a condition, and optionally, have already undergone treatment for an IL-17a-associated disease or disorder, an IL-17f-associated disease or disorder, or a TNF-α-associated disease or disorder or the one or more complications related to an IL-17a-associated disease or disorder, an IL-17f-associated disease or disorder, or a TNF-α-associated disease or disorder. Alternatively, a subject can also be one who has not been previously diagnosed as having an IL-17a-associated disease or disorder, an IL-17f-associated disease or disorder, or a TNF-α-associated disease or disorder or one or more complications related to an IL-17a-associated disease or disorder, an IL-17f-associated disease or disorder, or a TNF-α-associated disease or disorder. For example, a subject can be one who exhibits one or more risk factors for an IL-17a-associated disease or disorder, an IL-17f-associated disease or disorder, or a TNF-α-associated disease or disorder or one or more complications related to an IL-17a-associated disease or disorder, an IL-17f-associated disease or disorder, or a TNF-α-associated disease or disorder or a subject who does not exhibit risk factors.

A “subject in need” of treatment for a particular condition can be a subject having that condition, diagnosed as having that condition, or at risk of developing that condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. an IL-17a-associated disease or disorder, an IL-17f-associated disease or disorder, or a TNF-α-associated disease or disorder. The term “treating” includes reducing or alleviating at least one adverse effect or symptom of a condition, disease or disorder associated with an IL-17a-associated disease or disorder, an IL-17f-associated disease or disorder, or a TNF-α-associated disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A method of modulating IL-17a/IL-17a receptor-induced signaling in a cell, the method comprising contacting the cell with an effective amount of at least one compound selected from the group consisting of compounds (1)-(11) and Formulas (I)-(III).
2. The method of paragraph 1, wherein the at least one compound is selected from compounds (8), (9), and (10).
3. The method of paragraph 1, wherein the at least one compound is selected from Formulas (I), (II), and (III).
4. The method of paragraph 1, wherein the modulating comprises decreasing the IL-17a/IL-17a receptor signaling in the cell.
5. The method of any one of paragraphs 1-4, wherein the modulating is specific for IL-17a/IL-17a receptor signaling.
6. The method of any one of paragraphs 1-4, wherein TNF-α/TNF-α receptor, IL-10/IL-10 receptor, IL-25/IL-25R receptor, and/or IFN-gamma/IFN-gamma receptor signaling in the cell is not modulated.
7. A method of modulating TNF-α/TNF-α receptor-induced signaling in a cell, the method comprising contacting the cell with an effective amount of at least one compound selected from the group consisting of compounds (12)-(65) and Formulas (IV) and (V).
8. The method of paragraph 7, wherein the at least one compound is selected from compounds (19) and (39).
9. The method of paragraph 7, wherein the at least one compound is selected from Formulas (IV) and (V).
10. The method of paragraph 7, wherein the modulating comprises decreasing the TNF-α/TNF-α receptor signaling in the cell.
11. The method of any one of paragraphs 7-10, wherein the modulating is specific for TNF-α/TNF-α receptor signaling.
12. The method of any one of paragraphs 7-10, wherein IL-17a/IL-17a receptor, IL-10/IL-10 receptor, IL-25/IL-25R receptor, and/or IFN-gamma/IFN-gamma receptor signaling in the cell is not modulated.
13. A method of modulating immune signaling in a cell, the method comprising contacting the cell with an effective amount of at least one compound selected from the group consisting of compounds (1)-(65) and Formulas (I)-(V).
14. The method of paragraph 13, wherein the at least one compound is selected from compounds (8), (9), (10), (19), and (39).
15. The method of paragraph 13, wherein the at least one compound is selected from Formulas (I), (II), (III), (IV), and (V).
16. The method of paragraph 13, wherein the modulating comprises decreasing IL-17a/IL-17a receptor signaling in the cell.
17. The method of paragraph 13, wherein the modulating comprises decreasing TNF-α/TNF-α receptor signaling in the cell.
18. The method of any one of paragraphs 1-17, wherein the cell is an immune cell.
19. The method of any one of paragraphs 1-18, wherein the cell expresses an IL-17 receptor or a TNF receptor.
20. The method of paragraph 19, wherein the IL-17 receptor is IL-17RA and/or IL-17RC.
21. The method of paragraph 19, wherein the TNF receptor is TNFR1 and/or TNFR2.
22. The method of any one of paragraphs 1-21, wherein said modulating is in in vitro.
23. The method of any one of paragraphs 1-21, wherein said modulating is in in vivo.
24. The method of paragraph 23, wherein said modulating is in a subject in need of modulating an immune response.
25. A method of modulating an immune response in a subject, the method comprising administering to a subject in need thereof an effective amount of at least one compound selected from the group consisting of compounds (1)-(65) and Formulas (I)-(V).
26. The method of paragraph 25, wherein the at least one compound is selected from compounds (8), (9), (10), (19), and (39).
27. The method of paragraph 25, wherein the at least one compound is selected from Formulas (I), (II), (III), (IV), and (V).
28. The method of paragraph 24 or 25, wherein the subject has or is diagnosed with an IL-17a-associated disease or disorder or an IL-17f-associated disease or disorder.
29. The method of paragraph 28, wherein the IL-17a-associated disease or disorder is selected from the group consisting of: rheumatoid arthritis, psoriasis, multiple sclerosis, systemic lupus erythematosus, Crohn's disease (CD), uveitis, ulcerative colitis, asthma, Behçet's disease, and hyper IgE syndrome.
30. The method of paragraph 28, wherein the IL-17f-associated disease or disorder is recurrent urinary tract infections.
31. The method of paragraph 24 or 25, wherein the subject has or is diagnosed with a TNF-α-associated disease or disorder.
32. The method of paragraph 31, wherein the TNF-α-associated disease or disorder is selected from the group consisting of: rheumatoid arthritis, Crohn's disease, atherosclerosis, psoriasis, sepsis, diabetes, obesity, asthma, ulcerative colitis, ankylosing spondylitis, and moderate to severe plaque psoriasis.
33. The method of paragraph 24 or 25, wherein the subject has or is diagnosed with an inflammatory disease or disorder or an autoimmune disease.
34. A method of treating an inflammatory disease or disorder or an autoimmune disease, the method comprising administering to a subject in need thereof an effective amount of at least one compound selected from the group consisting of compounds (1)-(65) and Formulas (I)-(V).
35. The method of paragraph 34, wherein the at least one compound is selected from compounds (8), (9), (10), (19), and (39).
36. The method of paragraph 34, wherein the at least one compound is selected from Formulas (I), (II), (III), (IV), and (V).
37. The method of paragraph 33 or 34, wherein the inflammatory disease or disorder is acute or chronic.
38. The method of any one of paragraphs 33-27, wherein the inflammatory disease or disorder is selected from the group consisting of arthritis, asthma, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves' disease, gastrointestinal allergies, conjunctivitis, atherosclerosis, coronary artery disease, angina, and small artery disease.
39. The method of paragraph 33 or 34, wherein the autoimmune disease is selected from the group consisting of glomerulonephritis, Goodpasture's syndrome, necrotizing vasculitis, lymphadenitis, peri-arteritis nodosa, systemic lupus erythematosus, rheumatoid, arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barré syndrome, Hashimoto's thyroiditis, and cardiomyopathy.
40. A method of treating an IL-17a-associated disease or disorder, the method comprising administering to a subject in need thereof an effective amount of at least one compound selected from the group consisting of compounds (1)-(11) and Formulas (I)-(III).
41. The method of paragraph 40, wherein the at least one compound is selected from compounds (8), (9), and (10).
42. The method of paragraph 40, wherein the at least one compound is selected from Formulas (I), (II), and (III).
43. The method of paragraph 40, wherein the IL-17a-associated disease or disorder is selected from the group consisting of: rheumatoid arthritis, psoriasis, multiple sclerosis, systemic lupus erythematosus, Crohn's disease (CD), uveitis, ulcerative colitis, asthma, Behçet's disease, and hyper IgE syndrome.
44. A method of treating an IL-17f-associated disease or disorder, the method comprising administering to a subject in need thereof an effective amount of at least one compound selected from the group consisting of compounds (1)-(11) and Formulas (I)-(III).
45. The method of paragraph 44, wherein the at least one compound is selected from compounds (8), (9), and (10).
46. The method of paragraph 44, wherein the at least one compound is selected from Formulas (I), (II), and (III).
47. The method of paragraph 44, wherein the IL-17f-associated disease or disorder is recurrent urinary tract infections.
48. A method of treating a TNF-α-associated disease or disorder, the method comprising administering to a subject in need thereof an effective amount of at least one compound selected from the group consisting of compounds (12)-(65) and Formulas (IV) and (V).
49. The method of paragraph 48, wherein the at least one compound is selected from compounds (19) and (39).
50. The method of paragraph 48, wherein the at least one compound is selected from Formulas (IV) and (V).
51. The method of paragraph 48, wherein the TNF-α-associated disease or disorder is selected from the group consisting of: rheumatoid arthritis, Crohn's disease, atherosclerosis, psoriasis, sepsis, diabetes, obesity, asthma, ulcerative colitis, ankylosing spondylitis, and moderate to severe plaque psoriasis.
52. A method of treating a neurological disease or disorder, the method comprising administering to a subject in need thereof an effective amount of at least one compound selected from the group consisting of compounds (1)-(65) and Formulas (I)-(V).
53. The method of paragraph 52, wherein the at least one compound is selected from compounds (8), (9), (10), (19), and (39).
54. The method of paragraph 52, wherein the at least one compound is selected from Formulas (I), (II), (III), (IV), and (V).
55. The method of paragraph 52, wherein the neurological disease or disorder is selected from autism spectrum disorder, schizophrenia, depression, and bipolar disorder.
56. The method of any one of paragraphs 52-55, wherein the neurological disease or disorder is associated with abnormal behavioral phenotypes and/or abnormal cortical phenotypes in the central nervous system.
57. The method of any one of paragraphs 52-56, wherein the neurological disease or disorder is associated with an autism-like phenotype.
58. The method of any one of paragraphs 23-57 further comprising administering an anti-inflammatory agent to the subject.
59. The method of any one of paragraphs 23-58 further comprising administering an immunomodulatory agent to the subject.
60. The method of any one of paragraphs 23-59, wherein the compound is administered orally or parenterally to the subject.
61. A pharmaceutical composition comprising at least one compound selected from the group consisting of compounds (1)-(65) and Formulas (I)-(V) and a pharmaceutically acceptable carrier.
62. The pharmaceutical composition of paragraph 61, wherein the at least one compound is selected from compounds (8), (9), (10), (19), and (39).
63. The pharmaceutical composition of paragraph 61, wherein the at least one compound is selected from Formulas (I), (II), (III), (IV), and (V).
64. The pharmaceutical composition of paragraph 61, wherein the pharmaceutical composition is formulated in a form selected from the group consisting of tablets, gel capsules, sugar-coated tablets, syrups, suspensions, solutions, powders, lyophilized powders, granules, emulsions, lipid particles, polymeric particles, and controlled release compositions.
65. The pharmaceutical composition of any one of paragraphs 61-64, wherein the pharmaceutical composition is formulated for parenteral or oral administration.

The technology described herein is further illustrated by the following examples which in no way should be construed as being further limiting.

### EXAMPLES

### Example 1: Immune Modulatory Agents

A screening platform was used to identify a set of small molecules that specifically modulate activity of the IL-17 receptor (the IL-17RA-IL-17RC heterodimer), which responds to interleukin-17a (IL-17a). In order to identify those that selectively modulate IL-17 receptor, another cytokine receptor (TNF-a receptor) was used a counter-screen. Accordingly, described herein are also a set of small molecules that specifically modulate activity of the TNF-a receptor, which responds to TNF-a. For more details concerning the screening platform and associated methods, see e.g., International Patent Application PCT/US2020/062761, filed Dec. 10, 2021, the contents of which are incorporated herein by reference in its entirety. Tests can be performed to validate that the identified molecules can regulate primary immune cell function in vitro and in vivo.

### Example 2: Screening Platform to Identify Immune Modulatory Agents

Described herein is the screening platform that was used to identify the immune modulatory agents. Immune cells often exert their effects by secreting cytokines. Cytokines then bind to their cognate receptors expressed on the membranes of target cells, binding of which initiates a downstream signaling cascade leading to various biological effects. For example, T helper (Th) 17 cells, which are responsible for mounting immune responses against extracellular pathogens and fungi, mediate their effects by producing cytokines including interleukin-17a (IL-17a). Under pathological conditions, however, Th17 cells and IL-17a promote inflammatory and autoimmune diseases. Genome-wide association studies in humans have linked genes involved in Th17 cell differentiation and function (e.g., IL23R) with susceptibility to Crohn's disease, arthritis, and psoriasis. Consistent with these, blocking antibodies targeting IL-17a or its receptor (IL-17R) have also been developed, resulting in successful clinical trials with psoriatic patients. Based on these results, an anti-IL-17a antibody (e.g., secukinumab) has been used for the treatment of moderate-to-severe plaque psoriasis. Th17 cells also function as critical mediators, working in pregnant mice, to induce neurodevelopmental disorder-like phenotypes in offspring prenatally exposed to maternal immune activation (MIA). These MIA-associated neurodevelopmental phenotypes are mediated by activation of IL-17R in the developing fetal brain. Therefore, offspring are protected from developing abnormal behavioral and cortical phenotypes by either selective removing Th17 cell population or inhibiting IL-17a activity. While increased IL-17R activity during embryogenesis promotes behavioral abnormalities, studies indicate that increasing IL-17R activity in adulthood of MIA offspring leads to amelioration of behavioral symptoms, social behavioral phenotypes. See e.g., Wilke et al., Trends Immunol 32, 603-611 (2011); Duerr et al., Science 314, 1461-1463 (2006); Nair et al., Nat Genet 41, 199-204 (2009); Stahl et al., Nat Genet 42, 508-514 (2010); Miossec & Kolls, Nature reviews. Drug discovery 11, 763-776 (2012); Choi et al., Science 351, 933-939 (2016); Shin et al., Nature 549, 482-487 (2017); the contents of each of which of incorporated herein by reference in their entireties.

Therefore, modulating IL-17R activity with small molecules can have therapeutic value not only in controlling autoimmunity but also in preventing and/or therapeutically treating certain types of neurodevelopmental disorders. However, what have been developed are mostly blocking antibodies, whose main effects are to suppress activities of cytokines and their receptors. Small molecule modulators are advantageous as they can modulate cytokine-receptor activity bi-directionally. They also can be administered orally unlike protein biologics, and they are generally more stable.

To identify small molecule compounds that control IL-17R activity, a screening platform was used to monitor IL-17a binding to its receptor. Upon IL-17a binding to its receptor subunit A (IL-17RA), another receptor subunit IL-17RC is recruited, and these two subunits form a heterodimer. In the exemplary reporter system, a transcription factor (tTA) was tethered, via a protease cleavage site, to IL-17RA. In addition, a tobacco etch virus (TEV) protease was fused to the receptor subunit IL-17RC. Upon the IL-17a-dependent recruitment of IL-17RC-TEV to IL-17RA-tTA, the tTA is cleaved off from its membrane anchor IL-17RA. The released tTA then enters the nucleus and activates the reporter gene (e.g., in this case, GFP). The IL-17RA/RC reporter system responds highly selectively to IL-17a, but not to other related IL-17 family cytokines (except IL-17f).

Furthermore, the reporter gene (GFP) is activated in a dose-dependent manner by IL-17a or (to a lesser degree) IL-17f, but not by IL-17c. Thus, the screening platform has a high signal-to-background ratio and specificity that was used herein to identify small molecule and/or protein modulators that enhance or suppress IL-17a and its receptor binding (or TNF-α and its receptor binding).

The IL-17RA/IL-17RC reporter system was used to identify the IL-17a-specific immune modulatory agents, as described herein (see e.g., compounds (1)-(16)). Furthermore, the IL-17A systems and methods were applied to generate other cytokine/cytokine receptor reporter system (tTA fused onto one receptor subunit via a protease cleavage site, TEV fused to another receptor subunit, optimal linkers inserted between different proteins to ensure high signal-to-background ratio; e.g., for identifying TNF-α-specific immune modulatory agents). The screening platform provided herein was used to identify immune modulatory agents including, but not limited to, small molecules and proteins that modulate IL-17A or TNF-α cytokine signaling.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Sequences

In some embodiments of any of the aspects, a screening platform as described herein uses an amino acid sequence selected from at least one of SEQ ID NOs: 1-15 or an amino acid sequence that is at least 80%, at least 85%, at least 90%, at least 91%, at least 92%, at least 93%, at least 94%, at least 95%, at least 96%, at least 97%, at least 98%, at least 99%, or more, identical to at least one of SEQ ID NOs: 1-15 that maintains the same function.

SEQ ID NO: 1 (interleukin-17A precursor [Homo sapiens]; NCBI Reference
Sequence: NP_002181.1):
mtpgktslvs lllllsleai vkagitiprn pgcpnsedkn fprtvmvnln ihnrntntnp krssdyynrs tspwnlhrne
dperypsviw eakcrhlgci nadgnvdyhm nsvpiqqeil vlrrepphcp nsfrlekilv svgctcvtpi vhhva
SEQ ID NO: 2 (interleukin-17 receptor A isoform 1 precursor [Homo sapiens];
NCBI Reference Sequence: NP_055154.3):
mgaarsppsa vpgpllglll lllgvlapgg aslrlldhra lvcsqpglnc tvknstcldd swihprnltp sspkdlqiql
hfahtqqgdl fpvahiewtl qtdasilyle gaelsvlqln tnerlcvrfe flsklrhhhr rwrftfshfv vdpdqeyevt
vhhlpkpipd gdpnhqsknf lvpdceharm kvttpcmssg slwdpnitve tleahqlrvs ftlwnesthy qilltsfphm
enhscfehmh hipaprpeef hqrsnvtltl rnlkgccrhq vqiqpffssc lndclrhsat vscpempdtp epipdymplw
vywfitgisi llvgsvilli vcmtwrlagp gsekysddtk ytdglpaadl ippplkprkv wiiysadhpl yvdvvlkfaq
flltacgtev aldlleeqai seagvmtwvg rqkqemvesn skiivlcsrg trakwqallg rgapvrlrcd hgkpvgdlft
aamnmilpdf krpacfgtyv vcyfsevscd gdvpdlfgaa pryplmdrfe evyfriqdle mfqpgrmhrv gelsgdnylr
spggrqlraa ldrfrdwqvr cpdwfecenl ysaddqdaps ldeevfeepl lppgtgivkr aplvrepgsq aclaidplvg
eeggaavakl ephlqprgqp apqplhtlvl aaeegalvaa vepgpladga avrlalageg eacpllgspg agrnsvlflp
vdpedsplgs stpmaspdll pedvrehleg lmlslfeqsl scqaqggcsr pamvltdpht pyeeeqrqsv qsdqgyisrs
spqppeglte meeeeeeeqd pgkpalplsp edleslrslq rqllfrqlqk nsgwdtmgse segpsa
SEQ ID NO: 3 (interleukin-17 receptor A isoform 2 precursor [Homo sapiens];
NCBI Reference Sequence: NP_001276834.1)
mgaarsppsa vpgpllglll lllgvlapgg aslrlldhra lvcsqpglnc tvknstcldd swihprnltp sspkdlqiql
hfahtqqgdl fpvahiewtl qtdasilyle gaelsvlqln tnerlcvrfe flsklrhhhr rwrftfshfv vdpdqeyevt
vhhlpkpipd gdpnhqsknf lvpdceharm kvttpcmssg slwdpnitve tleahqlrvs ftlwnesthy
qilltsfphmenhscfehmh hipaprpeef hqrsnvtltl rnlkgccrhq vqiqpffssc lndclrhsat vscpempdtp
epipgpgsek ysddtkytdg lpaadlippp lkprkvwiiy sadhplyvdv vlkfaqfllt acgtevaldl leeqaiseag
vmtwvgrqkq emvesnskii vlcsrgtrak wqallgrgap vrlrcdhgkp vgdlftaamn milpdfkrpa cfgtyvvcyf
sevscdgdvp dlfgaapryp lmdrfeevyf riqdlemfqp grmhrvgels gdnylrspgg rqlraaldrf rdwqvrcpdw
fecenlysad dqdapsldee vfeepllppg tgivkraplv repgsqacla idplvgeegg aavaklephl qprgqpapqp
lhtlvlaace galvaavepg pladgaavrl alagegeacp llgspgagrn svlflpvdpe dsplgsstpm aspdllpedv
rehleglmls lfeqslscqa qggcsrpamv ltdphtpyee eqrqsvqsdq gyisrsspqp pegltemeee eeeeqdpgkp
alplspedle slrslqrqll frqlqknsgw dtmgsesegp sa
SEQ ID NO: 4 (interferon gamma precursor [Homo sapiens]); NCBI Reference Sequence:
NP_000610.2):
mkytsyilaf qlcivlgslg cycqdpyvke aenlkkyfna ghsdvadngt lflgilknwk eesdrkimqs qivsfyfklf
knfkddqsiq ksvetikedm nvkffnsnkk krddfekltn ysvtdlnvqr kaiheliqvm aelspaaktg krkrsqmlfr
grrasq
SEQ ID NO: 5 (IL17RA-linker-tTA (Mus musculus)):
MAIRRCWPRVVPGPALGWLLLLLNVLAPGRASPRLLDFPAPVCAQEGLSCRVKNST
CLDDSWIHPKNLTPSSPKNIYINLSVSSTQHGELVPVLHVEWTLQTDASIL YLEGAELS
VLQLNTNERLCVKFQFLSMLQHHRKRWRFSFSHFVVDPGQEYEVTVHHLPKPIPDG
DPNHKSKIIFVPDCEDSKMKMTTSCVSSGSLWDPNITVETLDTQHLRVDFTLWNEST
PYQVLLESFSDSENHSCFDVVKQIFAPRQEEFHQRANVTFTLSKFHWCCHHHVQVQP
FFSSCLNDCLRHAVTVPCPVISNTTVPKPVADYIPLWVYGLITLIAILLVGSVIVLIICM
TWRLSGADQEKHGLGSENLYFQYRLDKSKVINSALELLNEVGIEGLTTRKLAQKLGV
EQPTLYWHVKNKRALLDALAIEMLDRHHTHFCPLEGESWQDFLRNNAKSFRCALLS
HRDGAKVHLGTRPTEKQYETLENQLAFLCQQGFSLENALYALSAVGHFTLGCVLED
QEHQVAKEERETPTTDSMPPLLRQAIELFDHQGAEPAFLFGLELIICGLEKQLKCESGS
AYSRARTKNNYGSTIEGLLDLPDDDAPEEAGLAAPRLSFLPAGHTRRLSTAPPTDVSL
GDELHLDGEDVAMAHADALDDFDLDMLGDGDSPGPGFTPHDSAPYGALDMADFEF
EQMFTDALGIDEYGG
SEQ ID NO: 6 (IFNγR1-linker-tTA (Mus musculus)):
MGPQAAAGRMILLVVLMLSAKVGSGALTSTEDPEPPSVPVPTNVLIKSYNLNPVVC
WEYQNMSQTPIFTVQVKVYSGSWTDSCTNISDHCCNIYEQIMYPDVSAWARVKAKV
GQKESDYARSKEFLMCLKGKVGPPGLEIRRKKEEQLSVLVFHPEVVVNGESQGTMF
GDGSTCYTFDYTVYVEHNRSGEILHTKHTVEKEECNETLCELNISVSTLDSRYCISVD
GISSFWQVRTEKSKDVCIPPFHDDRKDSIWILVVAPLTVFTVVILVFAYWYTKKNSFK
RKSIMLLGSENLYFQYRLDKSKVINSALELLNEVGIEGLTTRKLAQKLGVEQPTLYW
HVKNKRALLDALAIEMLDRHHTHFCPLEGESWQDFLRNNAKSFRCALLSHRDGAKV
HLGTRPTEKQYETLENQLAFLCQQGFSLENALYALSAVGHFTLGCVLEDQEHQVAK
EERETPTTDSMPPLLRQAIELFDHQGAEPAFLFGLELIICGLEKQLKCESGSAYSRART
KNNYGSTIEGLLDLPDDDAPEEAGLAAPRLSFLPAGHTRRLSTAPPTDVSLGDELHLD
GEDVAMAHADALDDFDLDMLGDGDSPGPGFTPHDSAPYGALDMADFEFEQMFTDA
LGIDEYGG
SEQ ID NO: 7 (IL10RA-linker-tTA (Mus musculus)):
MLSRLLPFLVTISSLSLEFIAYGTELPSPSYVWFEARFFQHILHWKPIPNQSESTYYEVA
LKQYGNSTWNDIHICRKAQALSCDLTTFTLDLYHRSYGYRARVRAVDNSQYSNWTT
TETRFTVDEVILTVDSVTLKAMDGIIYGTIHPPRPTITPAGDEYEQVFKDLRVYKISIRK
FSELKNATKRVKQETFTLTVPIGVRKFCVKVLPRLESRINKAEWSEEQCLLITTEQYF
TVTNLSILVISMLLFCGILVCLVLQWYIRHPGKLPTVLVFKKPLGSENLYFQYRLDKS
KVINSALELLNEVGIEGLTTRKLAQKLGVEQPTLYWHVKNKRALLDALAIEMLDRH
HTHFCPLEGESWQDFLRNNAKSFRCALLSHRDGAKVHLGTRPTEKQYETLENQLAF
LCQQGFSLENALYALSAVGHFTLGCVLEDQEHQVAKEERETPTTDSMPPLLRQAIEL
FDHQGAEPAFLFGLELIICGLEKQLKCESGSAYSRARTKNNYGSTIEGLLDLPDDDAP
EEAGLAAPRLSFLPAGHTRRLSTAPPTDVSLGDELHLDGEDVAMAHADALDDFDLD
MLGDGDSPGPGFTPHDSAPYGALDMADFEFEQMFTDALGIDEYGG
SEQ ID NO: 8 (TNFαR1-linker-tTA (Homo sapiens)):
MGLSTVPDLLLPLVLLELLVGIYPSGVIGLVPHLGDREKRDSVCPQGKYIHPQNNSIC
CTKCHKGTYLYNDCPGPGQDTDCRECESGSFTASENHLRHCLSCSKCRKEMGQVEIS
SCTVDRDTVCGCRKNQYRHYWSENLFQCFNCSLCLNGTVHLSCQEKQNTVCTCHA
GFFLRENECVSCSNCKKSLECTKLCLPQIENVKGTEDSGTTVLLPLVIFFGLCLLSLLFI
GLMYRYQRWKSKLYSIVCGLGSENLYFQLRLDKSKVINSALELLNEVGIEGLTTRKL
AQKLGVEQPTLYWHVKNKRALLDALAIEMLDRHHTHFCPLEGESWQDFLRNNAKS
FRCALLSHRDGAKVHLGTRPTEKQYETLENQLAFLCQQGFSLENALYALSAVGHFTL
GCVLEDQEHQVAKEERETPTTDSMPPLLRQAIELFDHQGAEPAFLFGLELIICGLEKQ
LKCESGSAYSRARTKNNYGSTIEGLLDLPDDDAPEEAGLAAPRLSFLPAGHTRRLSTA
PPTDVSLGDELHLDGEDVAMAHADALDDFDLDMLGDGDSPGPGFTPHDSAPYGAL
DMADFEFEQMFTDALGIDEYGG
SEQ ID NO: 9 (IL17RC-linker-TEV (Mus musculus)):
MPVSWFLLSLALGRNPVVVSLERLMEPQDTARCSLGLSCHLWDGDVLCLPGSLQSA
PGPVLVPTRLQTELVLRCPQKTDCALCVRVVVHLAVHGHWAEPEEAGKSDSELQES
RNASLQAQVVLSFQAYPIARCALLEVQVPADLVQPGQSVGSAVEDCFEASLGAEVQI
WSYTKPRYQKELNLTQQLPDCRGLEVRDSIQSCWVLPWLNVSTDGDNVLLTLDVSE
EQDFSFLLYLRPVPDALKSLWYKNLTGPQNITLNHTDLVPCLCIQVWSLEPDSERVEF
CPFREDPGAHRNLWHIARLRVLSPGVWQLDAPCCLPGKVTLCWQAPDQSPCQPLVP
PVPQKNATVNEPQDFQLVAGHPNLCVQVSTWEKVQLQACLWADSLGPFKDDMLLV
EMKTGLNNTSVCALEPSGCTPLPSMASTRAARLGEELLQDFRSHQCMQLWNDDNM
GSLWACPMDKYIHRRWVLVWLACLLLAAALFFFLLLKKDRRKAARGSRTALGSSLF
KGPRDYNPISSTICHLTNESDGHTTSLYGIGFGPFIITNKHLFRRNNGTLLVQSLHGVF
KVKNTTTLQQHLIDGRDMIIIRMPKDFPPFPQKLKFREPQREERICLVTTNFQTKSMSS
MVSDTSCTFPSSDGIFWKHWIQTKDGQCGSPLVSTRDGFIVGIHSASNFTNTNNYFTS
VPKNFMELLTSQEAQQWVSGWRLNADSVLWGGHKVFMSKPEEPFQPVKEATQLM
NELVYSYPYDVPDYA
SEQ ID NO: 10 (IL17RB-linker-TEV (Mus musculus)):
MLLVLLILAASCRSALPREPTIQCGSETGPSPEWMVQHTLTPGDLRDLQVELVKTSV
AAEEFSILMNISWILRADASIRLLKATKICVSGKNNMNSYSCVRCNYTEAFQSQTRPS
GGKWTFSYVGFPVELSTLYLISAHNIPNANMNEDSPSLSVNFTSPGCLNHVMKYKKQ
CTEAGSLWDPDITACKKNEKMVEVNFTTNPLGNRYTILIQRDTTLGFSRVLENKLMR
TSVAIPVTEESEGAVVOLTPYLHTCGNDCIRREGTVVLCSETSAPIPPDDNRRMLGGW
LPLFLVLLVAVWVLAAGIYLTWRQGRSTKTSFPISGSSLFKGPRDYNPISSTICHLTNE
SDGHTTSLYGIGFGPFIITNKHLFRRNNGTLLVQSLHGVFKVKNTTTLQQHLIDGRDM
IIIRMPKDFPPFPQKLKFREPQREERICLVTTNFQTKSMSSMVSDTSCTFPSSDGIFWKH
WIQTKDGQCGSPLVSTRDGFIVGIHSASNFTNTNNYFTSVPKNFMELLTSQEAQQWV
SGWRLNADSVLWGGHKVFMSKPEEPFQPVKEATQLMNELVYSYPYDVPDYA
SEQ ID NO: 11 (IFNγR2-linker-TEV (Mus musculus)):
MRPLPLWLPSLLLCGLGAAASSPDSFSQLAAPLNPRLHLYNDEQILTWEPSPSSNDPR
PVVYQVEYSFIDGSWHRLLEPNCTDITETKCDLTGGGRLKLFPHPFTVFLRVRAKRG
NLTSKWVGLEPFQHYENVTVGPPKNISVTPGKGSLVIHFSPPFDVFHGATFQYLVHY
WEKSETQQEQVEGPFKSNSIVLGNLKPYRVYCLQTEAQLILKNKKIRPHGLLSNVSC
HETTANASARLQQVILIPLGIFALLLGLTGACFTLFLKYQSRVKYWFQAPPNIGSSLFK
GPRDYNPISSTICHLTNESDGHTTSLYGIGFGPFIITNKHLFRRNNGTLLVQSLHGVFK
VKNTTTLQQHLIDGRDMIIIRMPKDFPPFPQKLKFREPQREERICLVTTNFQTKSMSSM
VSDTSCTFPSSDGIFWKHWIQTKDGQCGSPLVSTRDGFIVGIHSASNFTNTNNYFTSV
PKNFMELLTSQEAQQWVSGWRLNADSVLWGGHKVFMSKPEEPFQPVKEATQLMNE
LVYSYPYDVPDYA
SEQ ID NO: 12 (IL10RB-linker-TEV (Mus musculus)):
MSWAPSVAGWLGGFLLVPALGMIPPPEKVRMNSVNFKNILQWEVPAFPKTNLTFTA
QYESYRSFQDHCKRTASTQCDFSHLSKYGDYTVRVRAELADEHSEWVNVTFCPVED
TIIGPPEMQIESLAESLHLRFSAPQIENEPETWTLKNIYDSWAYRVQYWKNGTNEKFQ
VVSPYDSEVLRNLEPWTTYCIQVQGFLLDQNRTGEWSEPICERTGNDEITPSWIVAIIL
IVSVLVVFLFLLGCFVVLWLIYKKTKHTFGSSLFKGPRDYNPISSTICHLTNESDGHTT
SLYGIGFGPFIITNKHLFRRNNGTLLVQSLHGVFKVKNTTTLQQHLIDGRDMIIIRMPK
DFPPFPQKLKFREPQREERICLVTTNFQTKSMSSMVSDTSCTFPSSDGIFWKHWIQTK
DGQCGSPLVSTRDGFIVGIHSASNFTNTNNYFTSVPKNFMELLTSQEAQQWVSGWRL
NADSVLWGGHKVFMSKPEEPFQPVKEATQLMNELVYSYPYDVPDYA
SEQ ID NO: 13 (TNFαR1-linker-TEV (Homo sapiens)):
MGLSTVPDLLLPLVLLELLVGIYPSGVIGLVPHLGDREKRDSVCPQGKYIHPQNNSIC
CTKCHKGTYLYNDCPGPGQDTDCRECESGSFTASENHLRHCLSCSKCRKEMGQVEIS
SCTVDRDTVCGCRKNQYRHYWSENLFQCFNCSLCLNGTVHLSCQEKQNTVCTCHA
GFFLRENECVSCSNCKKSLECTKLCLPQIENVKGTEDSGTTVLLPLVIFFGLCLLSLLFI
GLMYRYQRWKSKLYSIVCGGSSLFKGPRDYNPISSTICHLTNESDGHTTSLYGIGFGP
FIITNKHLFRRNNGTLLVQSLHGVFKVKNTTTLQQHLIDGRDMIIIRMPKDFPPFPQKL
KFREPQREERICLVTTNFQTKSMSSMVSDTSCTFPSSDGIFWKHWIQTKDGQCGSPLV
STRDGFIVGIHSASNFTNTNNYFTSVPKNFMELLTSQEAQQWVSGWRLNADSVLWG
GHKVFMSKPEEPFQPVKEATQLMNELVYSYPYDVPDYA
SEQ ID NO: 14 (TRE-H2B-GFP):
MPEPAKSAPAPKKGSKKAVTKAQKKGGKKRKRSRKESYSIYVYKVLKQVHPDTGIS
SKAMGIMNSFVNDIFERIAGEASRLAHYNKRSTITSREIQTAVRLLLPGELAKHAVSE
GTKAITKYTSAKDPPVATMVSKGEELFTGVVPILVELDGDVNGHKFSVSGEGEGDAT
YGKLTLKFICTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSALSK
DPNEKRDHMVLLEFVTAAGITLGMDELYK
SEQ ID NO: 15 (TRE-GFP):
MVSKGEELFTGVVPILVELDGDVNGHKFSVSGEGEGDATYGKLTLKFICTTGKLPVP
WPTLVTTLTYGVQCFSRYPDHMKQHDFFKSAMPEGYVQERTIFFKDDGNYKTRAEV
KFEGDTLVNRIELKGIDFKEDGNILGHKLEYNYNSHNVYIMADKQKNGIKVNFKIRH
NIEDGSVQLADHYQQNTPIGDGPVLLPDNHYLSTQSALSKDPNEKRDHMVLLEFVTA
AGITLGMDELYK

### Example 3

For details on reporter cell development, see e.g., International Patent Application PCT/US2021/062761, filed Dec. 10, 2021, published as WO2022125865A2 on Jun. 16, 2022, the contents of which are incorporated herein by reference in its entirety

### Screening Procedures

Two thousand reporter cells per well (384-well plates) were seeded in 30 ul cell culture medium with either IL-17A (25 ng/ml, unless otherwise indicated) or TNFa ligand (25 ng/ml, unless otherwise indicated); additional screens were performed with IL-17A at 7.5 ng/ml. Then 100 nl of the individual compound was added by a pin transfer and cultured for two days in a CO₂ incubator. For the cell viability test, 10 ul HOECHST 33342 was added and incubated for 10 min, followed by the addition of 10 ul 4% PFA. After a 10 min incubation, the reporter cells were washed twice with phosphate-buffered saline (PBS) and kept in 30 ul PBS. The images were acquired and analyzed using an IMAGEXPRESS confocal microscope. For storage, plates were sealed with black light-absorbing films.

### Screen History

1) We performed in parallel screens were performed using both IL-17Ra and TNFa reporters (total screened compound number: 134K)

A screen was performed with high ligand concentration (IL-17A: 25 ng/ml and TNFa 25 ng/ml); the library used was CHEMDIV 6, KNOWN BIOACTIVES AND CHEMBRIDGE 2020 (total compound number: 111K).

A screen was performed with low ligand concentration (IL-17A: 7.5 ng/mL and TNFa 12.5 ng/ml; the library used was ASINEX 2 (total compound number: 23K).

A total of 139 and 352 hits were identified as IL17A-R-specific and TNFaR-specific inhibitors, respectively (“Cherrypick”).

Among these “cherry-picked” compounds, 11 and 54 compounds were selected as L17A-specific and TNFaR-specific inhibitors (“confirmation stage”—as screened against other reporter cell lines). These compounds were not active against three additional reporter lines: IL10R/IL25R/IFNgR reporters.

Tables 9-12, below, show the IC₅₀ (μM) and Eₘₐₓ of compounds from FIGS. 2, 3, 6 and 7. As used herein the term IC₅₀ refers to the half-maximal inhibitory concentration, which is a measure of a compound's efficacy. IC₅₀ indicates how much of the compound is needed to inhibit a biological process by half (e.g., IL-17A receptor signaling or TNF-a receptor signaling), thus providing a measure of potency. The lower the value of IC₅₀ for a given compound, the higher the efficacy of the compound. As used herein the term Eₘₐₓ (also referred to as intrinsic activity or efficacy) refers to the maximal effect of the compound at high compound concentrations when all the receptors are occupied by the compound. The higher the value of Eₘₐₓ, the higher the efficacy of the compound. An Eₘₐₓ of 100% is an efficacy equal to the endogenous ligand (e.g., IL-17A or TNF-a). An Eₘₐₓ of between 0% and 100% is an efficacy equal less than the endogenous ligand.

TABLE 9
IC₅₀ and Eₘₐₓ of compounds reducing IL-17A-receptor reporter activities
(see e.g., FIG. 2, FIG. 9, FIG. 11). “Hits in FIG. 11” shows
that the compounds indicated in that column with an “X” (compounds
2, 3, 8, 9, 10) were confirmed to have inhibitory effects against some
of TNF-a downstream genes at the secondary assays (see e.g., FIG. 11).

| Compound # | IC₅₀ (μM) | Eₘₐₓ | Reference | hits in FIG. 11 |
|---|---|---|---|---|
| 1 | 4.813 | 78% | C770-0163 | |
| 2 | 31.39 | 82% | C770-0711 | X |
| 3 | 12.2 | 44% | C770-0720 | X |
| 4 | Undetermined | Undetermined | C770-0260 | |
| 5 | 6.825 | 57% | C770-0181 | |
| 6 | Undetermined | Undetermined | 60546479 | |
| 7 | 14.98 | 57% | K781-8036 | |
| 8 | 4.666 | 94% | C999-860 | X |
| 9 | 0.891 | 67% | C881-0413 | X |
| 10 | Undetermined | Undetermined | LAS 51595730 | X |
| 11 | Undetermined | Undetermined | LAS 51593897 | |

TABLE 10
IC₅₀ and Eₘₐₓ of compounds reducing TNF-a-receptor reporter activities (see
e.g., FIG. 3, FIG. 10, FIG. 12). “Hits in FIG. 12” shows that the
compounds indicated in that column with an “X” (compounds 15, 19,
21, 28, 32, 39, and 43) were confirmed to have inhibitory effects against
some of TNF-a downstream genes at the secondary assays (see e.g., FIG. 12).
“Derivatize” shows that the compounds indicated in that column with
an “X” (particularly C18 and C19) have amendable chemical structures for derivatization.

| Compound # | IC₅₀ (μM) | Eₘₐₓ | Reference | hits in FIG. 12 | derivatize |
|---|---|---|---|---|---|
| 12 | 6.302 | 32% | | | |
| 13 | 1.951 | 42% | | | |
| 14 | Undetermined | Undetermined | | | |
| 15 | Undetermined | Undetermined | c: LAS | X | |
| | | | 51649286 | | |
| 16 | 1.578 | 75% | | | |
| 17 | 1.458 | 66% | | | |
| 18 | Undetermined | Undetermined | | | |
| 19 | 7.464 | 98% | C6: D262- | X | X |
| | | | 1066 | | |
| 20 | Undetermined | Undetermined | | | |
| 21 | Undetermined | Undetermined | d: LAS | X | |
| | | | 52468797 | | |
| 22 | Undetermined | Undetermined | | | |
| 23 | 5.302 | 76% | | | |
| 24 | 0.610 | 75% | C18: | | X |
| | | | 79912002 | | |
| 25 | 1.667 | 52% | | | |
| 26 | Undetermined | Undetermined | | | |
| 27 | 2.163 | 66% | | | |
| 28 | Undetermined | Undetermined | a: LAS | X | |
| | | | 51613424 | | |
| 29 | Undetermined | Undetermined | | | |
| 30 | Undetermined | Undetermined | | | |
| 31 | Undetermined | Undetermined | | | |
| 32 | Undetermined | Undetermined | b: LAS | X | |
| | | | 51616998 | | |
| 33 | Undetermined | Undetermined | | | |
| 34 | 0.860 | 64% | C12: G832- | | X |
| | | | 0044 | | |
| 35 | 2.559 | 97% | | | |
| 36 | 7.719 | 92% | | | |
| 37 | Undetermined | Undetermined | | | |
| 38 | 3.987 | 91% | | | |
| 39 | 8.911 | 78% | C5: C509- | X | X |
| | | | 0242 | | |
| 40 | 2.158 | 76% | | | |
| 41 | 5.886 | 98% | | | |
| 42 | Undetermined | Undetermined | | | |
| 43 | Undetermined | Undetermined | e: LAS | X | |
| | | | 52108223 | | |
| 44 | Undetermined | Undetermined | | | |
| 45 | 1.303 | 67% | | | |
| 46 | 9.652 | 91% | | | |
| 47 | Undetermined | Undetermined | | | |
| 48 | 3.625 | 68% | | | |
| 49 | 0.791 | 87% | | | |
| 50 | Undetermined | Undetermined | | | |
| 51 | 0.823 | 85% | C19: | | X |
| | | | 23314788 | | |
| 52 | 8.453 | 90% | | | |
| 53 | Undetermined | Undetermined | | | |
| 54 | Undetermined | Undetermined | | | |
| 55 | Undetermined | Undetermined | | | |
| 56 | Undetermined | Undetermined | | | |
| 57 | 2.344 | 47% | | | |
| 58 | Undetermined | Undetermined | | | |
| 59 | Undetermined | Undetermined | | | |
| 60 | 6.984 | 88% | C7: G534- | | X |
| | | | 2460 | | |
| 61 | 8.776 | 88% | | | |
| 62 | 1.819 | 60% | | | |
| 63 | 1.777 | 65% | | | |
| 64 | 4.853 | 93% | | | |
| 65 | 2.963 | 41% | | | |

TABLE 11
IC₅₀ and Eₘₐₓ of compounds reducing IL-17A-receptor
reporter activities (see e.g., FIG. 6)

| Compound # | IC₅₀ (μM) | Eₘₐₓ |
|---|---|---|
| 1 | 4.813 | 78% |
| 2 | 31.39 | 82% |
| 3 | 12.2 | 44% |
| 66 | Undetermined | Undetermined |
| 67 | Undetermined | Undetermined |
| 68 | Undetermined | Undetermined |
| 4 | Undetermined | Undetermined |
| 5 | 6.825 | 57% |
| 69 | Undetermined | Undetermined |
| 70 | Undetermined | Undetermined |
| 71 | Undetermined | Undetermined |
| 8 | 4.666 | 94% |
| 6 | Undetermined | Undetermined |
| 9 | 0.891 | 67% |
| 7 | 14.98 | 57% |
| 72 | Undetermined | Undetermined |

TABLE 12
IC₅₀ and Eₘₐₓ of compounds reducing TNF-a-receptor
reporter activities (see e.g., FIG. 7)

| Compound # | IC₅₀ (μM) | Eₘₐₓ |
|---|---|---|
| 40 | 2.158 | 76% |
| 46 | 9.652 | 91% |
| 16 | 1.578 | 75% |
| 62 | 1.819 | 60% |
| 57 | 2.344 | 47% |
| 14 | Undetermined | Undetermined |
| 73 | Undetermined | Undetermined |
| 74 | 8.453 | 90% |
| 37 | Undetermined | Undetermined |
| 26 | Undetermined | Undetermined |
| 51 | 0.823 | 85% |
| 50 | Undetermined | Undetermined |
| 75 | 2.163 | 66% |
| 76 | 1.355 | 64% |
| 77 | 3.554 | 60% |
| 49 | 0.791 | 87% |
| 58 | Undetermined | Undetermined |
| 17 | 1.458 | 66% |
| 41 | 5.886 | 98% |
| 63 | 1.777 | 65% |
| 24 | 0.610 | 75% |
| 78 | Undetermined | Undetermined |
| 47 | Undetermined | Undetermined |
| 48 | 3.625 | 68% |
| 13 | 1.951 | 42% |
| 79 | 0.730 | 29% |
| 80 | Undetermined | Undetermined |
| 59 | Undetermined | Undetermined |
| 65 | 2.963 | 41% |
| 81 | Undetermined | Undetermined |
| 82 | 1.208 | 51% |
| 83 | Undetermined | Undetermined |
| 12 | 6.302 | 32% |
| 25 | 1.667 | 52% |

1. A method of modulating IL-17a/IL-17a receptor-induced signaling in a cell, the method comprising contacting the cell with an effective amount of at least one compound selected from the group consisting of compounds (1)-(11) and Formulas (I)-(III).

2. The method of claim 1, wherein the at least one compound is selected from compounds (8), (9), and (10); or the at least one compound is selected from Formulas (I), (II), and (III).

3. (canceled)

4. The method of claim 1, wherein the modulating comprises decreasing the IL-17a/IL-17a receptor signaling in the cell; wherein the modulating is specific for IL-17a/IL-17a receptor signaling; and/or wherein TNF-α/TNF-α receptor, IL-10/IL-10 receptor, IL-25/IL-25R receptor, and/or IFN-gamma/IFN-gamma receptor signaling in the cell is not modulated.

5-6. (canceled)

7. A method of modulating TNF-α/TNF-α receptor-induced signaling in a cell, the method comprising contacting the cell with an effective amount of at least one compound selected from the group consisting of compounds (12)-(65) and Formulas (IV) and (V).

8. The method of claim 7, wherein the at least one compound is selected from compounds (19) and (39); or the at least one compound is selected from Formulas (IV) and (V).

9. (canceled)

10. The method of claim 7, wherein the modulating comprises decreasing the TNF-α/TNF-α receptor signaling in the cell; wherein the modulating is specific for TNF-α/TNF-α receptor signaling; and/or wherein IL-17a/IL-17a receptor, IL-10/IL-10 receptor, IL-25/IL-25R receptor, and/or IFN-gamma/IFN-gamma receptor signaling in the cell is not modulated.

11-12. (canceled)

13. A method of modulating immune signaling in a cell, the method comprising contacting the cell with an effective amount of at least one compound selected from the group consisting of compounds (1)-(65) and Formulas (I)-(V); wherein the modulating comprises decreasing IL-17a/IL-17a receptor signaling in the cell or decreasing TNF-α/TNF-α receptor signaling in the cell.

14. The method of claim 13, wherein the at least one compound is selected from compounds (8), (9), (10), (19), and (39); or the at least one compound is selected from Formulas (I), (II), (III), (IV), and (V).

15-17. (canceled)

18. The method of claim 13, wherein the cell is an immune cell; wherein the cell expresses an IL-17 receptor selected from IL-17RA and/or IL-17RC, or the cell expresses a TNF receptor selected from TNFR1 and/or TNFR2.

19-21. (canceled)

22. The method of claim 1, wherein said modulating is in in vitro or said modulating is in in vivo.

23. (canceled)

24. The method of claim 22, wherein said modulating is in a subject in need of modulating an immune response.

25-27. (canceled)

28. The method of claim 24, wherein the subject has or is diagnosed with: an IL-17a-associated disease or disorder; an IL-17f-associated disease or disorder; a TNF-α-associated disease or disorder; an inflammatory disease or disorder; an autoimmune disease; or a neurological disease or disorder.

29. The method of claim 28, wherein the IL-17a-associated disease or disorder is selected from the group consisting of: rheumatoid arthritis, psoriasis, multiple sclerosis, systemic lupus erythematosus, Crohn's disease (CD), uveitis, ulcerative colitis, asthma, Behçet's disease, and hyper IgE syndrome.

30. The method of claim 28, wherein the IL-17f-associated disease or disorder is recurrent urinary tract infections.

31. (canceled)

32. The method of claim 28, wherein the TNF-α-associated disease or disorder is selected from the group consisting of: rheumatoid arthritis, Crohn's disease, atherosclerosis, psoriasis, sepsis, diabetes, obesity, asthma, ulcerative colitis, ankylosing spondylitis, and moderate to severe plaque psoriasis.

33-37. (canceled)

38. The method of claim 28, wherein the inflammatory disease or disorder is selected from the group consisting of arthritis, asthma, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves' disease, gastrointestinal allergies, conjunctivitis, atherosclerosis, coronary artery disease, angina, and small artery disease.

39. The method of claim 28, wherein the autoimmune disease is selected from the group consisting of glomerulonephritis, Goodpasture's syndrome, necrotizing vasculitis, lymphadenitis, peri-arteritis nodosa, systemic lupus erythematosus, rheumatoid, arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barré syndrome, Hashimoto's thyroiditis, and cardiomyopathy.

40-54. (canceled)

55. The method of claim 28, wherein the neurological disease or disorder is:
selected from autism spectrum disorder, schizophrenia, depression, and bipolar disorder;
associated with abnormal behavioral phenotypes and/or abnormal cortical phenotypes in the central nervous system; and/or
associated with an autism-like phenotype.

56-57. (canceled)

58. The method of claim 22 further comprising administering an anti-inflammatory agent or an immunomodulatory agent to the subject.

59-60. (canceled)

61. A pharmaceutical composition comprising at least one compound selected from the group consisting of compounds (1)-(65) and Formulas (I)-(V) and a pharmaceutically acceptable carrier.

62-65. (canceled)
